(19) United States
(12) Patent Application Publication
(10) Pub. No.: US 2026/0174959 A1
(43) Pub. Date: Jun. 25, 2026

(54) ANALYTE PATTERN ANALYSIS

(71) Applicant: Abbott Diabetes Care Inc., Alameda, CA (US)

(72) Inventors: Anup V. KANALE, Sunnyvale, CA (US); Tao PENG, San Ramon, CA (US); Nandita SINGH, Milpitas, CA (US); Jeffrey NISHIDA-BOUCHER, Redwood City, CA (US); Aparajita BHATTACHARYA, Dublin, CA (US); Byron Paul OLSON, II, Sioux Falls, SD (US); Gary A. HAYTER, Oakland, CA (US)

(21) Appl. No.: 19/295,270

(22) Filed: Aug. 8, 2025

Related U.S. Application Data
(60) Provisional application No. 63/737,875, filed on Dec. 23, 2024.

(51) Int. Cl. A61M 5/172; G16H 10/40; G16H 15/00; G16H 20/17; G16H 40/67; G16H 50/20

(52) U.S. Cl. CPC A61M 5/1723; G16H 10/40; G16H 15/00; G16H 20/17; G16H 40/67; G16H 50/20; A61M 2205/502; A61M 2205/6081; A61M 2230/201

(57) ABSTRACT

A glucose data analysis system includes an on-body unit and a processor. The on-body unit is configured to measure glucose levels of a user. The processor is configured to receive therapy data of the user including glucose data monitored by the on-body unit, analyze daily glucose profiles of the glucose data over an analysis period by a machine learning model, group the daily glucose profiles into two or more patterns based on the analysis performed by the machine learning model, and output a report including identification of the two or more patterns associated with each daily glucose profile. Advantageously the system may identify and visualize patterns of glucose data, identify recurring patterns, identify correlations between patterns and external events (e.g., medication, meals, exercise, sleep, stress, etc.), reduce glycemic variability, streamline analysis with unsupervised machine learning (e.g., no training), and provide recommendations for user intervention or actions based on identified patterns.

### CROSS-REFERENCE TO RELATED APPLICATIONS

This application claims the benefit of U.S. Provisional Application No. 63/737,875, filed Dec. 23, 2024, which is incorporated herein by reference in its entirety.

### FIELD

The present disclosure relates to analyte pattern analysis apparatuses, systems, and methods, for example, glucose pattern analysis apparatuses, systems, and methods for identifying and reporting recurring analyte patterns (e.g., glucose patterns) based on a pattern analysis model (e.g., a machine learning model).

### BACKGROUND

Monitoring analyte levels (e.g., glucose levels) can be important to the health and wellness of individuals (e.g., non-diabetic users, athletes, etc.), and can be vitally important to the health of individuals with diabetes. People with diabetes (PwD) are generally required to monitor their glucose levels to ensure they are maintained within a clinically safe range, and may also use this information to determine if or when insulin is needed to reduce glucose levels in their bodies or when additional glucose is needed to raise glucose levels in their bodies. A number of systems allow individuals to monitor their glucose levels, for example, continuous glucose monitoring (CGM).

Glycemic control is crucial for PwD. Elevated glucose levels (e.g., hyperglycemia) can lead to several complications including cardiovascular issues, kidney problems, and nerve damage, and low glucose levels (e.g., hypoglycemia) can impede a person's physical and mental activities including shaking, dizziness, and even hospitalization. Hence, it is important to maintain glucose levels within a certain range (e.g., between 70 to 180 mg/dL). However, many PwD exhibit large glycemic variability and day-to-day glucose deviations, even with prescribed therapies (e.g., oral medication, insulin dosing regimen, etc.), due to glucose levels being affected by multiple factors dependent upon daily routine and self-care (e.g., medication dosing, meals, exercise, stress, etc.). Thus, although therapy for PwD is generally based on an “average day,” this “average day” therapy is not effective when one's daily routine changes day-to-day.

Increased adoption of CGM sensors offers opportunities to provide individuals, both PwD and non-diabetic users, with personalized and optimized therapy treatments or wellness routines (e.g., diet, exercise routine, training regimen, etc.). Data from CGM sensors can capture an individual's analyte profile (e.g., glucose profile, ketones profile, lactate profile, etc.) over a period of days or weeks, which can provide insights into one's analyte trends and patterns (e.g., glucose patterns) both within a daily cycle and across multiple days. An Ambulatory Glucose Profile (AGP) is a CGM data analysis method to assess one's glucose trends and variations within the day.

However, current daily monitoring schemes (e.g., AGP) do not contemplate analyzing pattern variations from day-to-day. Further, current daily monitoring schemes do not consider different daily habits and routines of individuals to identify multiple distinct recurring patterns. Moreover, current reports of daily monitoring schemes (e.g., AGP report) do not identify or visualize distinct patterns in an individual's multi-day analyte data (e.g., glucose data).

### SUMMARY

Accordingly, aspects of the invention may provide an analyte pattern analysis system (e.g., glycemic pattern analysis system) that may identify and visualize distinct patterns of analyte data (e.g., glucose data). Further, aspects of the invention may provide an analyte pattern analysis system that may identify recurring analyte patterns (e.g., glucose patterns). Further, aspects of the invention may provide an analyte pattern analysis system that may identify correlations between analyte patterns (e.g., glucose patterns) and one or more external events (e.g., medication dosing, meals, exercise, sleep, stress, etc.). Further, aspects of the invention may provide an analyte pattern analysis system that may help reduce analyte variability (e.g., glycemic variability, ketones variability, lactate variability, etc.). Further, aspects of the invention may provide an analyte pattern analysis system that may output a report identifying and visually distinguishing two or more patterns associated with an individual's multi-day analyte data (e.g., glucose data). Further, aspects of the invention may provide an analyte pattern analysis system that may streamline pattern analysis with machine learning methods (e.g., unsupervised machine learning model). Further, aspects of the invention may provide an analyte pattern analysis system that may provide one or more recommendations for user intervention or user action based on identified patterns. Further, aspects of the invention may provide an analyte pattern analysis system that may develop personalized and optimized treatment for an individual.

Further, aspects of the invention may provide an analyte pattern analysis system that may develop personalized and optimized titration profiles for adaptive dose guidance for an individual. Further, aspects of the invention may provide an analyte pattern analysis system that may identify one or more patterns (e.g., weekdays vs. weekends, day shift vs. night shift, not traveling vs. traveling, etc.) and generate one or more titration profiles for each of the identified patterns. Further, aspects of the invention may provide an analyte pattern analysis system that utilizes a generative artificial intelligence (AI) model to determine whether analyte patterns are correlated to a first type of user data (e.g., weekdays, weekends, day shift, night shift, not traveling, traveling, etc.). Further, aspects of the invention may provide an analyte pattern analysis system that may utilize a large language model (LLM) to compare identified analyte patterns to one or more types of user data to determine a level of correlation.

Further, aspects of the invention may provide an analyte pattern analysis system that may develop personalized and optimized titration profiles for adaptive dose guidance during pregnancy. Further, aspects of the invention may provide an analyte pattern analysis system that may develop personalized and optimized titration profiles for adaptive dose guidance during menstruation. Further, aspects of the invention may provide an analyte pattern analysis system that may develop personalized and optimized titration profiles for adaptive dose guidance during illness. Further, aspects of the invention may provide an analyte pattern analysis system that may identify two or more patterns (e.g., pregnancy vs. non-pregnancy, menstruation vs. non-menstruation, illness vs. non-illness, etc.) and generate two or more titration profiles each corresponding to one of the identified patterns.

In some aspects, a method of identifying and visualizing patterns of analyte data may include monitoring, by an analyte monitoring device, analyte levels of a user. In some aspects, the analyte monitoring device may include a first portion arranged above a skin surface of the user, and a second portion arranged below the skin surface and in contact with interstitial fluid of the user. In some aspects, the method may further include receiving, by at least one processor, therapy data of the user. In some aspects, the therapy data may include analyte data monitored by the analyte monitoring device. In some aspects, the method may further include analyzing, by the at least one processor, analyte profiles of the analyte data over an analysis period by a model. The analyte profile may be a daily analyte profile. The analyte profile may be a glucose profile. The analyte profile may be a daily glucose profile. The analyte profile may be a ketones profile. The analyte profile may be a daily ketones profile. The analyte profile may be a lactate profile. The analyte profile may be a daily lactate profile. The model may be a machine learning model. The model may be an unsupervised machine learning model (e.g., no training). In some aspects, the method may further include identifying, by the at least one processor, each of the analyte profiles as corresponding to a pattern of two or more patterns based on the analysis performed by the model. In some aspects, the method may further include outputting, on a display device in communication with the at least one processor, a report including identification of the two or more patterns associated with each analyte profile.

In some aspects, the report may include a display of each analyte profile. In some aspects, the display overlays the two or more patterns over the analysis period and visually distinguishes the patterns from one another. In some aspects, the display visually distinguishes the patterns from one another by color.

In some aspects, the report may include a calendar with an indication of an identified pattern of the two or more patterns for each day on the calendar. In some aspects, the calendar separates the two or more patterns into corresponding calendar days and visually distinguishes the patterns from one another. In some aspects, the calendar visually distinguishes the patterns from one another by color.

In some aspects, the analysis of the analyte profiles may include assessing a distance between the analyte profiles. In some aspects, the distance may include a mean absolute relative difference (MARD). In some aspects, first and second analyte profiles may be grouped into a first pattern type when the distance is at or below a threshold. In some aspects, first analyte profile is grouped into the first pattern type and the second analyte profile is grouped into a second pattern type when the distance is above the threshold.

In some aspects, the method may further include determining one or more insights (e.g., of the user) based on the report. The insights may include information such as interpretations, observations, conclusions, understandings, recommendations (including for user interventions and/or actions), notifications, and/or alerts, which may be based on the analyte profiles, the analysis, and/or the identified patterns. In some aspects, the one or more insights may include one or more recurring analyte patterns. The recurring analyte patterns may be recurring daily analyte patterns. In some aspects, the one or more insights may include one or more of a time-of-day analyte variation, a day-to-day analyte variation, elevated analyte times, elevated analyte days, a weekday variation, a weekday-to-weekend variation, a medication dosing variation, a mealtime variation, an activity variation, or a combination thereof. In some aspects, the one or more insights may include a time-of-day analyte variation (e.g., a time-of-day glucose variation). In some aspects, the one or more insights may include a day-to-day analyte variation (e.g., a day-to-day glucose variation). In some aspects, the one or more insights may include elevated analyte times (e.g., elevated glucose times). In some aspects, the one or more insights may include elevated analyte days (e.g., elevated glucose days). In some aspects, the one or more insights may include a weekday variation. In some aspects, the one or more insights may include a weekday-to-weekend variation. In some aspects, the one or more insights may include a medication dosing variation. In some aspects, the one or more insights may include a mealtime variation. In some aspects, the one or more insights may include an activity variation. In some aspects, the method may further include providing the one or more insights in a message to the user, a health care professional, or both, for example, on a receiver device or a remote device, or both.

In some aspects, each of the analyte profiles includes analyte data of the user that is collected over a 24-hour time window. The analyte profile may be a daily analyte profile based on analyte data collected over the 24-hour time window.

In some aspects, each of the analyte profiles includes analyte data of the user that is collected over less than a 24-hour time window.

In some aspects, an analyte data analysis system may include an on-body unit and at least one processor. In some aspects, the on-body unit may be configured to be worn on a skin surface of a user. In some aspects, the on-body unit may include an analyte sensor and sensor electronics. In some aspects, the analyte sensor may be configured to measure analyte levels in the body of the user. In some aspects, the analyte sensor may include a first portion arranged above the skin surface, and a second portion arranged below the skin surface and in contact with interstitial fluid of the user. In some aspects, the sensor electronics may be coupled to the analyte sensor and configured to wirelessly transmit analyte data. In some aspects, the at least one processor may be in wireless communication with the on-body unit. In some aspects, the at least one processor may be coupled to at least one memory storing instructions that when executed by the at least one processor cause the at least one processor to perform operations including receiving therapy data of the user. In some aspects, the therapy data may include analyte data monitored by the on-body unit. In some aspects, the operations may further include analyzing analyte profiles of the analyte data over an analysis period. The analyte profile may be a daily analyte profile. The analyte profile may be a glucose profile. The analyte profile may be a daily glucose profile. The analyte profile may be a ketones profile. The analyte profile may be a daily ketones profile. The analyte profile may be a lactate profile. The analyte profile may be a daily lactate profile. The analysis may be performed by a model. The model may be a machine learning model. In some aspects, the operations may further include identifying each of the analyte profiles as corresponding to a pattern of two or more patterns based on the analysis performed by the model. In some aspects, the operations may further include outputting a report including identification of the two or more patterns associated with each analyte profile.

In some aspects, the report may include a display of each analyte profile. In some aspects, the report may include a calendar with an indication of an identified pattern of the two or more patterns for each day on the calendar.

In some aspects, the machine learning model may include unsupervised machine learning.

In some aspects, a computer-readable storage medium storing instructions which, when executed by one or more processors, may cause the one or more processors to perform operations including receiving therapy data of a user. In some aspects, the therapy data may include analyte data monitored by an analyte monitoring device. In some aspects, the analyte monitoring device may include a first portion arranged above a skin surface of the user, and a second portion arranged below the skin surface and in contact with interstitial fluid of the user. In some aspects, the operations may further include analyzing analyte profiles of the analyte data over an analysis period by a model. The analyte profile may be a daily analyte profile. The analyte profile may be a glucose profile. The analyte profile may be a daily glucose profile. The analyte profile may be a ketones profile. The analyte profile may be a daily ketones profile. The analyte profile may be a lactate profile. The analyte profile may be a daily lactate profile. The model may be a machine learning model. The model may be an unsupervised machine learning model (e.g., no training). In some aspects, the operations may further include identifying each of the analyte profiles as corresponding to a pattern of two or more patterns based on the analysis performed by the model. In some aspects, the operations may further include outputting a report including identification of the two or more patterns associated with each analyte profile.

In some aspects, the report may include a graph of the two or more patterns. In some aspects, the graph represents a mapping of the two or more patterns onto a plane and visually distinguishes the patterns from one another. The graph may be a scatter plot of the two or more patterns. In some aspects, the graph visually distinguishes the patterns from one another by color. In some aspects, the graph is based at least in part on a day-to-day analyte variation between each of the analyte profiles.

In some aspects, the therapy data may include medication data. The therapy data may include medication dosing data. In some aspects, the method or operations may further include detecting, by a pen cap releasably coupleable to a manual injection pen and in communication with the at least one processor, a dosing event of the manual injection pen. In some aspects, the dosing event may be inferred from a decapping event of the pen cap from the manual injection pen and a capping event of the pen cap to the manual injection pen.

In some aspects, the therapy data may include meal data. In some aspects, the method or operations may further include detecting, by the at least one processor or a smart device in communication with the at least one processor, a meal event of the user.

In some aspects, the therapy data may include one or more of activity data, exercise data, stress data, sleep data, location data, travel data, calendar data, or a combination thereof. In some aspects, the therapy data may include activity data. In some aspects, the therapy data may include exercise data. In some aspects, the therapy data may include stress data. In some aspects, the therapy data may include sleep data. In some aspects, the therapy data may include location data. In some aspects, the therapy data may include travel data. In some aspects, the therapy data may include calendar data. In some aspects, the method or operations may further include detecting, by the at least one processor or one or more smart devices in communication with the at least one processor, one or more of an activity event of the user, an exercise event of the user, a stress event of the user, a sleep event of the user, a location event of the user, a travel event of the user, or a combination thereof. In some aspects, the method or operations may further include detecting an activity event of the user. In some aspects, the method or operations may further include detecting an exercise event of the user. In some aspects, the method or operations may further include detecting a stress event of the user. In some aspects, the method or operations may further include detecting a sleep event of the user. In some aspects, the method or operations may further include detecting a location event of the user. In some aspects, the method or operations may further include detecting a travel event of the user.

In some aspects, each of the analyte profiles may include analyte data segments. In some aspects, the analyte data segments may be based on events, such as meals. In some aspects, the analyte data segments may include one or more of a breakfast time window, a lunch time window, a dinner time window, a snack time window, or a combination thereof, among other windows.

In some aspects, the analyzing may include generating, by the at least one processor, a distance matrix based on a distance between two daily analyte profiles. In some aspects, the analyzing may include generating, by the at least one processor, a distance matrix based on a distance between pairs of the analyte profiles. In some aspects, the distance matrix may be an N×N symmetric matrix representing the distance between each pair of N analyte profiles.

In some aspects, the distance may be a mean absolute difference (MAD). For example, the MAD may be defined by the average or mean (e.g., expected value E) of the absolute difference of two independent sequences X and Y (e.g., |X−Y|), each drawn from an analyte profile (e.g., a daily analyte profile), as represented by Equation (1) below:

$$MAD = E [ \vert X - Y \vert ] = \frac{1}{n} {\sum }_{i = 1}^{n} \vert {x}_{i} - {y}_{i} \vert \qquad ( 1 )$$

for a random sample of size n of a population.

In some aspects, the distance may be a weighted MAD (WMAD). For example, the WMAD may be defined by the MAD in which some data points count (e.g., are weighted) more heavily than others in the calculation, as represented by Equation (2) below:

$$WMAD = \frac{1}{\sum {w}_{i}} \frac{1}{n} {\sum }_{i = 1}^{n} {w}_{i} \vert {x}_{i} - {y}_{i} \vert \qquad ( 2 )$$

where the weights w sum to one for a random sample of size n of a population.

In some aspects, the distance may be a mean absolute relative difference (MARD). For example, the MARD may be defined by the MAD divided by the arithmetic mean (AM), which quantifies the MAD in comparison to the size of the mean for measurement accuracy as a single (dimensionless) numeric value, as represented by Equation (3) below:

$$MARD = \frac{2}{n} {\sum }_{i = 1}^{n} \frac{\vert {x}_{i} - {y}_{i} \vert}{{x}_{i} + {y}_{i}} \qquad ( 3 )$$

for a random sample of size n of a population.

In some aspects, the identifying may include constructing a point set from the distance matrix. In some aspects, each point of the point set may represent an analyte profile.

In some aspects, the constructing the point set may include multidimensional scaling of the distance matrix. In some aspects, the multidimensional scaling may include translating elements of the distance matrix to the point set in a plane (e.g., a scatter plot) such that such that a distance between two points of the point set is the distance between a corresponding pair of the analyte profiles.

In some aspects, the identifying may further include performing cluster analysis on the point set. In some aspects, the performing cluster analysis may include performing k-means clustering. In some aspects, the performing cluster analysis may include performing a Gaussian mixture model.

In some aspects, the performing cluster analysis may include utilizing a metric to identify the two or more patterns from each other. In some aspects, the metric may include a distance reduction ratio metric based on a separation between each pattern. In some aspects, the distance reduction ratio metric may be defined as a ratio between an average inter-pattern distance to an average intra-pattern distance, as represented by Equation (4) below:

$$R = \frac{{avg}_{( i , j ) ∈ S 1} d ( i , j )}{{avg}_{u ∈ S 1 and v ∈ S 2} {d}^{′} ( u , v )} \qquad ( 4 )$$

where d(i, j) represents the inter-pattern distance between two analyte profiles i and j, both belonging to the same pattern S1, and d′(u, v) represents the intra-pattern distance of two analyte profiles u and v, from two different patterns S1 and S2 respectively.

In some aspects, the distance reduction ratio metric may include a threshold at or above which the two or more patterns are identified. In some aspects, the threshold is at least about 20%. In some aspects, the threshold is at least about 25%. In some aspects, the threshold is at least about 30%. In some aspects, the threshold is in a range from about 20% to about 30%. In some aspects, the threshold is in a range from about 15% to about 40%.

In some aspects, the metric may include a meal metric based on a separation between each pattern in relation to meal data of the user.

In some aspects, the metric may include a medication metric based on a separation between each pattern in relation to medication data of the user. In some aspects, the metric may include a medication dosing metric based on a separation between each pattern in relation to medication dosing data of the user.

In some aspects, the analyzing and the identifying may include utilizing the model to compare the analyte profiles and identify the two or more patterns. The model may include a machine learning model.

In some aspects, the machine learning model may include unsupervised machine learning.

In some aspects, the machine learning model may include supervised machine learning.

In some aspects, the method or operations may further include correlating the two or more patterns to one or more user parameters based on one or more metrics. In some aspects, the one or more user parameters may include one or more of medication data statistics, medication dosing data statistics, meal data statistics, activity data statistics, exercise data statistics, stress data statistics, sleep data statistics, location data statistics, travel data statistics, calendar data statistics, daily routine statistics, or a combination thereof. In some aspects, the one or more user parameters may include medication data statistics. In some aspects, the one or more user parameters may include medication dosing data statistics. In some aspects, the one or more user parameters may include meal data statistics. In some aspects, the one or more user parameters may include activity data statistics. In some aspects, the one or more user parameters may include exercise data statistics. In some aspects, the one or more user parameters may include stress data statistics. In some aspects, the one or more user parameters may include sleep data statistics. In some aspects, the one or more user parameters may include location data statistics. In some aspects, the one or more user parameters may include travel data statistics. In some aspects, the one or more user parameters may include calendar data statistics. In some aspects, calendar data statistics may include meeting times, start and end times, durations, event types, dates, time of day, or a combination thereof. In some aspects, the one or more user parameters may include daily routine statistics. In some aspects, daily routine statistics may include or be based on CGM application use and/or sensor use. In some aspects, for example, daily routine statistics may include a number of scans, how often a user checks CGM reports, how often a user checks their glucose levels, or a combination thereof. In some aspects, the one or more metrics may include one or more of a statistical metric, a distance reduction ratio metric, a meal metric, a medication metric, a medication dosing metric, or a combination thereof. In some aspects, the one or more metrics may include a statistical metric. In some aspects, the one or more metrics may include a distance reduction ratio metric. In some aspects, the one or more metrics may include a meal metric. In some aspects, the one or more metrics may include a medication metric. In some aspects, the one or more metrics may include a medication dosing metric.

In some aspects, the method or operations may further include providing a recommendation to the user or a health care professional based at least in part on the two or more patterns.

In some aspects, the method or operations may further include providing a recommendation to the user or a health care professional based on a correlation of the two or more patterns to the one or more user parameters.

In some aspects, the analysis period may be 3 or more days, 5 or more days, 7 or more days, 15 or more days, and may be in a range from 3 days to 15 days, 5 days to 15 days, 7 days to 15 days, or 14 days to 30 days. In some aspects, the identifying may be limited to no greater than three patterns. For example, for a shorter analysis period (e.g., 15 days or less), the identifying may be limited to no greater than three patterns since for daily analyte profiles the pattern detection system (model) is only considering 15 data points (e.g., n=15) and more than three patterns may not be statistically significant.

In some aspects, the analysis period may be at least 14 days. In some aspects, the analysis period may be at least 15 days. In some aspects, the analysis period may be in a range from 3 days to 30 days. In some aspects, the analysis period may be at least 28 days. In some aspects, the analysis period may be at least 30 days. In some aspects, the analysis period may be in a range from 30 days to 90 days. In some aspects, the analysis period may be at least 45 days. For example, for a longer analysis period (e.g., greater than 15 days, greater than 30 days, greater than 45 days, etc.), the identifying may include three or more patterns (e.g., three patterns, four patterns, five patterns, etc.) since the pattern detection system (model) is considering a larger number of data points (e.g., n>15) and may thereby identify more patterns from the larger data set.

In some aspects, the system may further include a pen cap releasably coupleable to an injection pen (e.g., manual insulin pen, manual GLP-1 pen, etc.) and configured to detect a dosing event of the injection pen. In some aspects, the dosing event may be inferred from a decapping event of the pen cap from the injection pen and a capping event of the pen cap to the injection pen. In some aspects, the dosing event may include information about the medication type (e.g., Metformin, prandial insulin, basal insulin), dose amount, and/or time of dose.

In some aspects, the system may further include a smart device in communication with the at least processor and configured to detect a meal event of the user.

In some aspects, the system may further include one or more smart devices in communication with the at least one processor and configured to detect one or more of an activity event of the user, an exercise event of the user, a stress event of the user, a sleep event of the user, a location event of the user, a travel event of the user, or a combination thereof.

In some aspects, a glucose data analysis system includes an on-body unit and a processor. The on-body unit is configured to measure glucose levels of a user. The processor is configured to receive therapy data of the user including glucose data monitored by the on-body unit, analyze daily glucose profiles of the glucose data over an analysis period by a machine learning model, group the daily glucose profiles into two or more patterns based on the analysis performed by the machine learning model, and output a report including identification of the two or more patterns associated with each daily glucose profile. Advantageously the system may identify and visualize patterns of glucose data, identify recurring patterns, identify correlations between patterns and external events (e.g., medication, meals, exercise, sleep, stress, etc.), reduce glycemic variability, streamline analysis with unsupervised machine learning (e.g., no training), and provide recommendations for user intervention or actions based on identified patterns.

In some aspects, a method of generating titration profiles for adaptive dose guidance may include monitoring, by a glucose monitoring device, glucose levels of a user. In some aspects, the glucose monitoring device may include a first portion arranged above a skin surface of the user, and a second portion arranged below the skin surface and in contact with interstitial fluid of the user. In some aspects, the method may further include receiving, by at least one processor in communication with the glucose monitoring device, user data of the user. In some aspects, the user data may include glucose data monitored by the glucose monitoring device. In some aspects, the method may further include analyzing, by the at least one processor, a plurality of glucose profiles of the glucose data over an analysis period. In some aspects, the method may further include grouping, by the at least one processor, the plurality of glucose profiles into two or more patterns based on the analysis. In some aspects, the method may further include generating, by the at least one processor, a plurality of titration profiles. In some aspects, each titration profile may correspond to one of the two or more patterns identified. In some aspects, the method may further include adjusting, by the at least one processor, one or more insulin therapy settings of a medication delivery device based on a titration profile of the plurality of titration profiles. In some aspects, the method may further include outputting, on a display, a dose recommendation based on the titration profile.

In some aspects, the method may further include dynamically updating, by the at least one processor, a titration profile of the plurality of titration profiles in real time based on a variability of the titration profile over time.

In some aspects, the two or more patterns identified may include weekdays and weekends. In some aspects, the plurality of titration profiles may include a first titration profile corresponding to weekdays and a second titration profile corresponding to weekends.

In some aspects, the two or more patterns identified may include a day shift and a night shift. In some aspects, the plurality of titration profiles may include a first titration profile corresponding to a day shift and a second titration profile corresponding to a night shift. In some aspects, the second titration profile may be shifted later in time relative to the first titration profile.

In some aspects, the two or more patterns identified may include not traveling and traveling. In some aspects, the plurality of titration profiles may include a first titration profile corresponding to not traveling and a second titration profile corresponding to traveling. In some aspects, the second titration profile may be shifted in time relative to the first titration profile based on a current location of the user.

In some aspects, the method may further include generating, by the at least one processor, a second titration profile based on one of the plurality of titration profiles when a variability of the titration profile is above a threshold.

In some aspects, the analyzing may be performed by a machine learning model. In some aspects, the machine learning model may include an unsupervised machine learning model performing cluster analysis.

In some aspects, the analyzing may be performed by a generative AI model. In some aspects, the method may further include prompting the generative AI model to determine whether the plurality of glucose profiles are correlated to weekdays or weekends. In some aspects, the method may further include prompting the generative AI model to determine whether the plurality of glucose profiles are correlated to a day shift or a night shift. In some aspects, the method may further include prompting the generative AI model to determine whether the plurality of glucose profiles are correlated to not traveling or traveling.

In some aspects, the method may further include prompting the generative AI model to determine whether the plurality of glucose profiles are correlated to a first type of user data. In some aspects, the method may further include prompting the generative AI model to determine whether the two or more patterns are correlated to a second type of user data. In some aspects, the prompting is performed by a LLM that compares the two or more patterns to the second type of user data to determine a level of correlation.

In some aspects, a pattern analysis system may include a glucose monitoring device and at least one processor in communication with the glucose monitoring device. In some aspects, the glucose monitoring device is configured to be worn on a skin surface of a user. In some aspects, the glucose sensor is configured to measure glucose levels of the user. In some aspects, the glucose sensor includes a first portion arranged above the skin surface, and a second portion arranged below the skin surface and in contact with interstitial fluid of the user. In some aspects, sensor electronics are coupled to the glucose sensor and are configured to wirelessly transmit glucose data. In some aspects, the at least one processor is coupled to at least one memory storing instructions that when executed cause the at least one processor to perform operations including receiving user data of the user. In some aspects, the user data may include glucose data monitored by the glucose monitoring device. In some aspects, the operations further include analyzing a plurality of glucose profiles of the glucose data over an analysis period. In some aspects, the operations further include grouping the plurality of glucose profiles into two or more patterns based on the analysis. In some aspects, the operations further include generating a plurality of titration profiles. In some aspects, each titration profile corresponds to one of the two or more patterns identified. In some aspects, the operations further include adjusting one or more insulin therapy settings of a medication delivery device based on a titration profile of the plurality of titration profiles. In some aspects, the operations further include outputting on a display a dose recommendation based on the titration profile.

In some aspects, the operations further include dynamically updating a titration profile of the plurality of titration profiles in real time based on a variability of the titration profile over time.

In some aspects, the analysis is performed by a machine learning model. In some aspects, the machine learning model may include an unsupervised machine learning model performing cluster analysis.

In some aspects, the analysis is performed by a generative AI model.

In some aspects, a computer-readable storage medium storing instructions which, when executed by at least one processor, cause the at least one processor to perform operations including receiving user data of a user. In some aspects, the user data may include glucose data monitored by a glucose monitoring device. In some aspects, the glucose monitoring device may include a first portion arranged above a skin surface of the user, and a second portion arranged below the skin surface and in contact with interstitial fluid of the user. In some aspects, the operations further include analyzing a plurality of glucose profiles of the glucose data over an analysis period. In some aspects, the operations further include grouping the glucose profiles into two or more patterns based on the analysis. In some aspects, the operations further include generating a plurality of titration profiles. In some aspects, each titration profile corresponds to one of the two or more patterns identified. In some aspects, the operations further include adjusting one or more insulin therapy settings of a medication delivery device based on a titration profile of the plurality of titration profiles. In some aspects, the operations further include outputting on a display a dose recommendation based on the titration profile.

In some aspects, the operations further include dynamically updating a titration profile of the plurality of titration profiles in real time based on a variability of the titration profile over time.

In some aspects, the method or operations may further include updating, by the at least one processor, the titration profile. In some aspects, the updating is performed in real time based on glucose data monitored over a second analysis period. In some aspects, the updating is based on a variability of one or more parameters of the titration profile exceeding a threshold. In some aspects, the one or more parameters of the titration profile comprises a dose amount, an insulin sensitivity factor (ISF), or a carbohydrate-to-insulin ratio (CR).

In some aspects, a method of generating titration profiles for adaptive dose guidance for pregnancy may include monitoring, by a glucose monitoring device, glucose levels of a user. In some aspects, the glucose monitoring device may include a first portion arranged above a skin surface of the user, and a second portion arranged below the skin surface and in contact with interstitial fluid of the user. In some aspects, the method may further include receiving, by at least one processor in communication with the glucose monitoring device, user data of the user. In some aspects, the user data may include glucose data monitored by the glucose monitoring device and pregnancy data regarding whether the user is pregnant or not pregnant. In some aspects, the method may further include analyzing, by the at least one processor, a plurality of glucose profiles of the glucose data and the pregnancy data over an analysis period. In some aspects, the method may further include grouping, by the at least one processor, the plurality of glucose profiles into a first pattern corresponding to the user being pregnant and a second pattern corresponding to the user not being pregnant based on the analysis. In some aspects, the method may further include generating, by the at least one processor, a first titration profile corresponding to the user being pregnant based on the first pattern and a second titration profile corresponding to the user not being pregnant based on the second pattern.

In some aspects, the method may further include dynamically updating, by the at least one processor, the first titration profile in real time based on a variability of the first titration profile over time.

In some aspects, the first titration profile may have a higher insulin dose range than the second titration profile.

In some aspects, the method may further include outputting a query to the user regarding whether the user is pregnant or not pregnant. In some aspects, the pregnancy data may be received from the user based on a response to the query.

In some aspects, the analyzing may be performed by a generative AI model. In some aspects, the method may further include prompting the generative AI model to determine whether the plurality of glucose profiles are correlated to the user being pregnant or the user not being pregnant.

In some aspects, a pattern analysis system for adaptive dose guidance for pregnancy may include a glucose monitoring device and at least one processor in communication with the glucose monitoring device. In some aspects, the glucose monitoring device is configured to be worn on a skin surface of a user. In some aspects, the glucose monitoring device may include a glucose sensor and sensor electronics. In some aspects, the glucose sensor is configured to measure glucose levels of the user. In some aspects, the glucose sensor may include a first portion arranged above the skin surface, and a second portion arranged below the skin surface and in contact with interstitial fluid of the user. In some aspects, the sensor electronics are coupled to the glucose sensor and configured to wirelessly transmit glucose data. In some aspects, the at least one processor is coupled to at least one memory storing instructions that when executed cause the at least one processor to perform operations including receiving user data of the user. In some aspects, the user data may include glucose data monitored by the glucose monitoring device and pregnancy data regarding whether the user is pregnant or not pregnant. In some aspects, the operations may further include analyzing a plurality of glucose profiles of the glucose data and the pregnancy data over an analysis period. In some aspects, the operations may further include grouping the plurality of glucose profiles into first pattern corresponding to the user being pregnant and a second pattern corresponding to the user not being pregnant based on the analysis. In some aspects, the operations may further include generating a first titration profile corresponding to the user being pregnant based on the first pattern and a second titration profile corresponding to the user not being pregnant based on the second pattern.

In some aspects, the operations may further include dynamically updating the first titration profile in real time based on a variability of the first titration profile over time.

In some aspects, the first titration profile may have a higher insulin dose range than the second titration profile.

In some aspects, the method or operations may further include updating, by the at least one processor, the first titration profile. In some aspects, the updating is performed in real time based on glucose data and pregnancy data monitored over a second analysis period. In some aspects, the updating is based on a variability of one or more parameters of the first titration profile exceeding a threshold. In some aspects, the one or more parameters of the first titration profile comprises a dose amount, an insulin sensitivity factor (ISF), a carbohydrate-to-insulin ratio (CR), or a total body weight of the user.

In some aspects, a method of generating titration profiles for adaptive dose guidance for menstruation may include monitoring, by a glucose monitoring device, glucose levels of a user. In some aspects, the glucose monitoring device may include a first portion arranged above a skin surface of the user, and a second portion arranged below the skin surface and in contact with interstitial fluid of the user. In some aspects, the method may further include receiving, by at least one processor in communication with the glucose monitoring device, user data of the user. In some aspects, the user data may include glucose data monitored by the glucose monitoring device and menstruation data regarding whether the user is menstruating or not menstruating. In some aspects, the method may further include analyzing, by the at least one processor, a plurality of glucose profiles of the glucose data and the menstruation data over an analysis period. In some aspects, the method may further include grouping, by the at least one processor, the plurality of glucose profiles into a first pattern corresponding to the user menstruating and a second pattern corresponding to the user not menstruating based on the analysis. In some aspects, the method may further include generating, by the at least one processor, a first titration profile corresponding to the user menstruating based on the first pattern and a second titration profile corresponding to the user not menstruating based on the second pattern.

In some aspects, the method may further include dynamically updating, by the at least one processor, the first titration profile in real time based on a variability of the first titration profile over time.

In some aspects, the first titration profile may have a higher insulin dose range and/or a lower insulin sensitivity factor (ISF) than the second titration profile.

In some aspects, the method may further include outputting a query to the user regarding whether the user is menstruating or not menstruating. In some aspects, the menstruation data is received from the user based on a response to the query.

In some aspects, the analyzing may be performed by a generative AI model. In some aspects, the method may further include prompting the generative AI model to determine whether the plurality of glucose profiles are correlated to the user menstruating or the user not menstruating.

In some aspects, a pattern analysis system for adaptive dose guidance for menstruation may include a glucose monitoring device and at least one processor in communication with the glucose monitoring device. In some aspects, the glucose monitoring device is configured to be worn on a skin surface of a user. In some aspects, the glucose monitoring device may include a glucose sensor and sensor electronics coupled to the glucose sensor. In some aspects, the glucose sensor is configured to measure glucose levels of the user. In some aspects, the glucose sensor may include a first portion arranged above the skin surface, and a second portion arranged below the skin surface and in contact with interstitial fluid of the user. In some aspects, the sensor electronics are configured to wirelessly transmit glucose data. In some aspects, the at least one processor is coupled to at least one memory storing instructions that when executed cause the at least one processor to perform operations including receiving user data of the user. In some aspects, the user data may include glucose data monitored by the glucose monitoring device and menstruation data regarding whether the user is menstruating or not menstruating. In some aspects, the operations may further include analyzing a plurality of glucose profiles of the glucose data and the menstruation data over an analysis period. In some aspects, the operations may further include grouping the plurality of glucose profiles into first pattern corresponding to the user menstruating and a second pattern corresponding to the user not menstruating based on the analysis. In some aspects, the operations may further include generating a first titration profile corresponding to the user menstruating based on the first pattern and a second titration profile corresponding to the user not menstruating based on the second pattern.

In some aspects, the operations further include dynamically updating the first titration profile in real time based on a variability of the first titration profile over time.

In some aspects, the first titration profile may have a higher insulin dose range and a lower ISF than the second titration profile.

In some aspects, the method or operations may further include updating, by the at least one processor, the first titration profile. In some aspects, the updating is performed in real time based on glucose data and menstruation data monitored over a second analysis period. In some aspects, the updating is based on a variability of one or more parameters of the first titration profile exceeding a threshold. In some aspects, the one or more parameters of the first titration profile comprises a dose amount, an insulin sensitivity factor (ISF), a carbohydrate-to-insulin ratio (CR), or a current menstrual phase of the user.

In some aspects, a method of generating titration profiles for adaptive dose guidance for illness may include monitoring, by a glucose monitoring device, glucose levels of a user. In some aspects, the glucose monitoring device may include a first portion arranged above a skin surface of the user, and a second portion arranged below the skin surface and in contact with interstitial fluid of the user. In some aspects, the method may further include receiving, by at least one processor in communication with the glucose monitoring device, user data of the user. In some aspects, the user data may include glucose data monitored by the glucose monitoring device and illness data regarding whether the user is ill or not ill. In some aspects, the method may further include analyzing, by the at least one processor, a plurality of glucose profiles of the glucose data and the illness data over an analysis period. In some aspects, the method may further include grouping, by the at least one processor, the plurality of glucose profiles into a first pattern corresponding to the user being ill and a second pattern corresponding to the user not being ill based on the analysis. In some aspects, the method may further include generating, by the at least one processor, a first titration profile corresponding to the user being ill based on the first pattern and a second titration profile corresponding to the user not being ill based on the second pattern.

In some aspects, the method may further include dynamically updating, by the at least one processor, the first titration profile in real time based on a variability of the first titration profile over time.

In some aspects, the first titration profile may have a higher insulin dose range than the second titration profile.

In some aspects, the first titration profile may have a lower insulin dose range than the second titration profile.

In some aspects, the method may further include outputting a query to the user regarding whether the user is ill or not ill. In some aspects, the illness data is received from the user based on a response to the query. In some aspects, the illness data may include body temperature data (e.g., fever) based on one or more monitoring devices.

In some aspects, the illness data may include a type of illness.

In some aspects, the analyzing may be performed by a generative AI model. In some aspects, the method may further include prompting the generative AI model to determine whether the plurality of glucose profiles are correlated to the user being ill or the user not being ill.

In some aspects, a pattern analysis system for adaptive dose guidance for illness may include a glucose monitoring device and at least one processor in communication with the glucose monitoring device. In some aspects, the glucose monitoring device is configured to be worn on a skin surface of a user. In some aspects, the glucose monitoring device may include a glucose sensor and sensor electronics coupled to the glucose sensor. In some aspects, the glucose sensor is configured to measure glucose levels of the user. In some aspects, the glucose sensor may include a first portion arranged above the skin surface, and a second portion arranged below the skin surface and in contact with interstitial fluid of the user. In some aspects, the sensor electronics are configured to wirelessly transmit glucose data. In some aspects, the at least one processor is coupled to at least one memory storing instructions that when executed cause the at least one processor to perform operations including receiving user data of the user. In some aspects, the user data may include glucose data monitored by the glucose monitoring device and illness data regarding whether the user is ill or not ill. In some aspects, the operations may further include analyzing a plurality of glucose profiles of the glucose data and the illness data over an analysis period. In some aspects, the operations may further include grouping the plurality of glucose profiles into first pattern corresponding to the user being ill and a second pattern corresponding to the user not being ill based on the analysis. In some aspects, the operations may further include generating a first titration profile corresponding to the user being ill based on the first pattern and a second titration profile corresponding to the user not being ill based on the second pattern.

In some aspects, the method or operations may further include updating, by the at least one processor, the first titration profile. In some aspects, the updating is performed in real time based on glucose data and illness data monitored over a second analysis period. In some aspects, the updating is based on a variability of one or more parameters of the first titration profile exceeding a threshold. In some aspects, the one or more parameters of the first titration profile comprises a dose amount, an insulin sensitivity factor (ISF), a carbohydrate-to-insulin ratio (CR), or a body temperature of the user.

In some aspects, a method of identifying and reporting patterns of glucose data includes monitoring, by a glucose monitoring device, glucose levels of a user. In some aspects, the glucose monitoring device may include a first portion arranged above a skin surface of the user, and a second portion arranged below the skin surface and in contact with interstitial fluid of the user. In some aspects, the method may further include receiving, by at least one processor in communication with the glucose monitoring device, therapy data of the user, wherein the therapy data comprises glucose data monitored by the glucose monitoring device. In some aspects, the method may further include analyzing, by the at least one processor, a plurality of daily glucose profiles of the glucose data over an analysis period. In some aspects, the method may further include grouping, by the at least one processor, the daily glucose profiles into two or more patterns based on the analysis. In some aspects, the method may further include outputting, on a display device in communication with the at least one processor, a report comprising identification of one of the two or more patterns associated with each daily glucose profile. In some aspects, the report includes a display of each daily glucose profile. In some aspects, the display overlays the two or more patterns over the analysis period and visually distinguishes each of the two or more patterns from another by color. In some aspects, the report further includes a calendar with an indication of one of the two or more patterns associated with the daily glucose profile for each day on the calendar.

In some aspects, the method may further include generating, by the at least one processor, a plurality of titration profiles. In some aspects, each titration profile corresponds to one of the two or more patterns identified. In some aspects, the method may further include adjusting, by the at least one processor, one or more insulin therapy settings of a medication delivery device based on a titration profile of the plurality of titration profiles. In some aspects, the one or more insulin therapy settings may include one or more of a dose amount, an insulin sensitivity factor (ISF), or a carbohydrate-to-insulin ratio (CR). In some aspects, the method may further include outputting, on the display device, a dose recommendation based on the titration profile.

In some aspects, a method of triggering an update to a titration profile for adaptive dose guidance may include monitoring, by a glucose monitoring device, glucose levels of a user. In some aspects, the glucose monitoring device may include a first portion arranged above a skin surface of the user, and a second portion arranged below the skin surface and in contact with interstitial fluid of the user. In some aspects, the method may further include receiving, by at least one processor in communication with the glucose monitoring device, user data of the user. In some aspects, the user data may include glucose data monitored by the glucose monitoring device. In some aspects, the method may further include updating, by the at least one processor, a titration profile of the user based on a variability of one or more parameters of the titration profile exceeding a threshold.

In some aspects, the one or more parameters of the titration profile may include a dose amount, an insulin sensitivity factor (ISF), and a carbohydrate-to-insulin ratio (CR). In some aspects, the updating may include updating based on a change in user data. In some aspects, the change in user data may include a change in pregnancy data, a change in menstruation data, a change in illness data, or a combination thereof.

Implementations of any of the techniques described above may include a system, a method, a process, a device, and/or an apparatus. The details of one or more implementations are set forth in the accompanying drawings and the description below. Other features will be apparent from the description and drawings, and from the claims.

Further features and example aspects of the present disclosure, as well as the structure and operation of various aspects, are described in detail below with reference to the accompanying drawings. It is noted that the aspects are not limited to the specific aspects described herein. Such aspects are presented herein for illustrative purposes only. Additional aspects will be apparent to persons skilled in the relevant art(s) based on the teachings contained herein.

### BRIEF DESCRIPTION OF THE DRAWINGS/FIGURES

The accompanying drawings, which are incorporated herein and form part of the specification, illustrate the aspects and, together with the description, further serve to explain the principles of the aspects and to enable a person skilled in the relevant art(s) to make and use the aspects.

FIG. 1 is a schematic illustration of an analyte monitoring system, according to an example aspect.

FIG. 2A illustrates a block diagram of a sensor control device, according to an example aspect.

FIG. 2B illustrates a block diagram of a sensor control device, according to an example aspect.

FIG. 3 illustrates a block diagram of a receiver device, according to an example aspect.

FIG. 4 is a schematic illustration of an analyte pattern analysis system, according to an example aspect.

FIG. 5 illustrates a flow diagram for the analyte pattern analysis system shown in FIG. 4, according to an example aspect.

FIGS. 6A-6D are schematic illustrations of a model for identifying and visualizing analyte patterns, according to an example aspect.

FIG. 7 shows a plot of daily glucose profiles as a function of time visually indicating three patterns, according to an example aspect.

FIG. 8 shows a plot of daily glucose profiles as a function of calendar day visually indicating three patterns, according to an example aspect.

FIG. 9 shows a plot of daily glucose profiles as a function of day-to-day glucose variation visually indicating two patterns, according to an example aspect.

FIGS. 10A-10C are schematic illustrations of a model for identifying analyte patterns and generating titration profiles, according to an example aspect.

FIG. 11 illustrates a flow diagram for the model shown in FIGS. 10A-10C, according to an example aspect.

FIGS. 12A-12C are schematic illustrations of a model for identifying analyte patterns and generating titration profiles, according to an example aspect.

FIG. 13 illustrates a flow diagram for the model shown in FIGS. 12A-12C to generate titration profiles for pregnancy, according to an example aspect.

FIG. 14 illustrates a flow diagram for the model shown in FIGS. 12A-12C to generate titration profiles for menstruation, according to an example aspect.

FIG. 15 illustrates a flow diagram for the model shown in FIGS. 12A-12C to generate titration profiles for illness, according to an example aspect.

The features and example aspects of the present disclosure will become more apparent from the detailed description set forth below when taken in conjunction with the drawings, in which like reference characters identify corresponding elements throughout. In the drawings, like reference numbers generally indicate identical, functionally similar, and/or structurally similar elements. Additionally, generally, the left-most digit(s) of a reference number identifies the drawing in which the reference number first appears. Unless otherwise indicated, the drawings provided throughout the disclosure should not be interpreted as to-scale drawings.

### DETAILED DESCRIPTION

Provided herein are system, apparatus, device, method, process, and/or computer program product aspects, and/or combinations and sub-combinations thereof, for identifying and visualizing recurring patterns of analyte data.

A system as described below may include an on-body unit configured to monitor analyte data of a user. The system includes a processor configured to analyze analyte profiles of the analyte data. The analyte profiles may be daily analyte profiles. The daily analyte profiles may be based on a calendar day or a period of about 24 hours. The analyte profiles are grouped into patterns. The grouping may be performed by a machine learning model. The system may output a report indicating the identified patterns for the analyte profiles.

A method as described below may include monitoring analyte data of a user with an analyte monitoring device. The method may include analyzing analyte profiles of the analyte data by a machine learning model. The analyte profiles may be daily analyte profiles (e.g., daily glucose profiles). The method may include grouping the analyte profiles into two or more patterns based on the analysis of the machine learning model. The method may include outputting a report on a display device identifying the pattern for each analyte profile and visually distinguishing the two or more patterns from another.

A machine learning model as described below may include one or more of comparing analyte profiles of analyte data of a user (e.g., daily glucose profiles), generating a distance matrix based on a distance between pairs of the analyte profiles, constructing a point set from the distance matrix by multidimensional scaling, and performing cluster analysis on the point set to identify two or more patterns of the analyte profiles.

Machine learning models may include, by way of example and not limitation, models trained using or encompassing decision tree analysis, gradient boosting, adaptive boosting, artificial neural networks or variants thereof, linear discriminant analysis, nearest neighbor analysis, support vector machines, supervised or unsupervised classification, and others. The models may also include algorithmic or rules-based models in addition to machine learned models. Machine learning involves computers discovering how they may perform tasks without being explicitly programmed to do so. Machine learning includes, but is not limited to, artificial intelligence, deep learning, fuzzy learning, supervised learning, and unsupervised learning, etc.

Machine learning algorithms may build an initial prediction model based on sample data, known as “training data”, in order to make predictions or decisions without being explicitly programmed to do so. This sample data may include analyte data (e.g., glucose data) and/or medication data (e.g., insulin data) from the user or from a population of users. For supervised learning, the computer is presented with example inputs and their desired outputs, and the goal is to learn a general rule that maps the inputs to the outputs. In another example, for unsupervised learning, no labels are given to the machine learning algorithm, leaving it on its own to find structure in the inputs. Unsupervised learning may be the ultimate goal, such as discovering trends or patterns in data, or as a means towards another goal, such as improved accuracy of future predictions.

A machine learning engine may use various classifiers to map concepts or data to identify relationships between concepts and an accuracy of prior predicted user outcomes. A classifier (discriminator) may be trained to distinguish (recognize) variations in the data. In some aspects, machine learning models are trained on a remote machine learning platform using historical data of the user, or from a population of user. In one aspect, prediction models may be continuously updated as new user information is received.

In some aspects, the first step in training a machine learning model to identify and report recurring analyte patterns is the acquisition of a dataset. The dataset may originate from various sources, such as databases, files, application programming interfaces (APIs), or real-time sensor feeds (e.g., on-body unit). For example, the dataset may include analyte data (e.g., glucose data) and/or medication data (e.g., insulin data) from the user or from a population of users. The dataset comprises multiple records, where each record includes one or more features (independent variables). Optionally, each record may include an associated label (dependent variable or target). For example, this target may be used for supervised machine learning tasks, while unlabeled data may be used for unsupervised machine learning tasks. In particular, analyte data of a user may be labeled into analyte profiles (e.g., date and times, daily glucose profiles), a distance matrix may be generated based on comparison between pairs of the analyte profiles using a distance metric (e.g., MARD), a point set may be constructed based on the distance matrix (e.g., via multidimensional scaling), and two or more patterns of the analyte data may be identified by cluster analysis, and the two or more patterns may then be labeled for training data. In some aspects, the two or more patterns may be known and the analyte data may be labeled as such to generate training data.

In some aspects, once the data is collected, it may undergo a pre-processing phase to ensure that it is clean and suitable for training. For example, the pre-processing may include data cleaning (e.g., removing or imputing missing values, eliminating duplicate entries, and/or correcting inconsistencies in the dataset), data normalization or scaling (e.g., transforming feature values to ensure they are on the same scale, which may improve model performance in certain algorithms), categorical encoding (e.g., converting categorical variables into numerical form, for example, one-hot encoding, for algorithms that require numeric input), and/or data splitting (e.g., dividing the dataset into two or more subsets, for example, training and testing sets). In some aspects, a portion of the data may be used for model training, and the remaining portion may be reserved for model evaluation to prevent overfitting (e.g., model performing well on training data but poorly on new data).

In some aspects, relevant features may be selected or engineered from the dataset to improve model performance. Feature selection techniques may be used to identify which attributes (features) have the most influence on the target variable. In some aspects, feature selection techniques may include MARD. Feature engineering may also create new features through transformations or combinations of existing ones. In some aspects, feature engineering techniques may include feature splitting (e.g., dividing the feature into two or more sub-features), handling outliers (e.g., omitting data points outside of a threshold or target range), or a combination thereof. This ensures that the model is provided with informative and non-redundant data so that training is efficient.

In some aspects, a machine learning model may be selected based on the nature of the problem and the characteristics of the data. In particular, a machine learning model as described below may identify recurring analyte patterns (e.g., glucose patterns) for reporting the identified analyte patterns (e.g., in a visually distinguishing way), hence an unsupervised machine learning model may be used. Suitable unsupervised machine learning algorithms may include cluster analysis, k-means clustering, hierarchical clustering, or a Gaussian mixture model. In particular, k-means clustering may be used to identify two or more patterns in the dataset. The selected algorithm defines the structure of the model, which is then initialized with staring parameters. In some aspects, the starting parameters may be an untrained model using analyte data over a period of time (e.g., at least 3 days). In some aspects, the staring parameters may be a partially trained model, in some cases, based on generalized data of a user or a population of users.

In some aspects, a training phase may involve feeding the training dataset into the chosen machine learning algorithm, allowing the model to learn patterns and relationships between the input features (e.g., daily analyte profiles) and the output target (e.g., identifying a pattern for each analyte profile), to identify and report recurring analyte patterns. During this process, the algorithm may iteratively adjust its internal parameters (e.g., weights, coefficients, metrics, and the like) to minimize the error between its predictions and the actual outcomes (e.g., optimization). In some aspects, for supervised machine learning, a loss function may be used to quantify the difference between predicted and actual values, and the model's parameters may be optimized to minimize this loss function. In some aspects, for unsupervised machine learning, the model may attempt to discover inherent patterns or groupings within the dataset.

In some aspects, after training, the model's performance may be evaluated using a separate testing dataset that was not seen during training (e.g., new analyte data outside of training data). The model's predictions may be compared against the actual outcomes using various evaluation metrics. In particular, a distance matrix may be generated based on a distance metric (e.g., MARD). In some aspects, for classification tasks, metrics such as accuracy, precision, recall, F1-score, or confusion matrix may be used. In some aspects, for clustering tasks, metrics such as a silhouette score, Davies-Bouldin index, or Dunn index may be used to assess the quality of the clusters. In some aspects, to avoid overfitting (e.g., where the model performs well on training data but poorly on new unseen data), techniques such as cross-validation or regularization may be employed.

In some aspects, to improve model performance, hyperparameters (e.g., predefined settings that control the machine learning process, such as learning rate or number of estimators) may be used and fine-tuned. This process may involve manual tuning, grid search, or random search methods. Cross-validation may be used in this step to ensure that the tuned model generalizes well to new data.

In some aspects, once the model achieves satisfactory performance, it is ready for deployment. Deployment refers to integrating the trained model into an application or system where it can make predictions on new, unseen data. The data input to the trained model generally mirrors the data used to train the model. In particular, the data input for the machine learning model may be daily analyte profiles of a user. The output of the trained model identifies and reports recurring analyte patterns (e.g., two or more patterns of the analyte profiles), for example, in a report visually distinguishing the two or more patterns from another (e.g., graph overlaying each daily analyte profile, on a calendar, etc.). In some aspects, the model may be exposed as an API, embedded into a software application, or used in batch processing pipelines. In some aspects, the model may be deployed on a mobile app. The deployed model may be monitored over time to ensure it continues to perform as expected and does not degrade due to changes in input data or system environments.

In some aspects, as new data becomes available, the model may be retrained periodically to maintain or improve its performance. Retraining may be initiated based on temporal intervals (e.g., every 6-months), performance thresholds, or changes in the data distribution. In some cases, models may be continuously updated in real-time (e.g., online learning) to adapt to new data.

This specification discloses one or more aspects that incorporate the features of this present invention.

The aspect(s) described, and references in the specification to “one aspect,” “an aspect,” “an example aspect,” “an exemplary aspect,” etc., indicate that the aspect(s) described may include a particular feature, structure, or characteristic, but every aspect may not necessarily include the particular feature, structure, or characteristic. Moreover, such phrases are not necessarily referring to the same aspect. Further, when a particular feature, structure, or characteristic is described in connection with an aspect, it is understood that it is within the knowledge of one skilled in the art to affect such feature, structure, or characteristic in connection with other aspects whether or not explicitly described.

The term “about” or “substantially” or “approximately” as used herein means the value of a given quantity that may vary based on a particular technology. Based on the particular technology, the term “about” or “substantially” or “approximately” may indicate a value of a given quantity that varies within, for example, 0.1-10% of the value (e.g., ±0.1%, ±1%, ±2%, ±5%, or ±10% of the value).

Numerical values, including endpoints of ranges, may be expressed herein as approximations preceded by the term “about,” “substantially,” “approximately,” or the like. In such cases, other aspects include the particular numerical values. Regardless of whether a numerical value is expressed as an approximation, two aspects are included in this disclosure: one expressed as an approximation, and another not expressed as an approximation. It will be further understood that an endpoint of each range is significant both in relation to another endpoint, and independently of another endpoint.

Aspects of the disclosure may be implemented in hardware, firmware, software, or any combination thereof. Aspects of the disclosure may also be implemented as instructions stored on a machine-readable medium (e.g., memory), which may be read and executed by one or more processors. A machine-readable medium may include any mechanism for storing or transmitting information in a form readable by a machine (e.g., a computing device). For example, a machine-readable medium may include read only memory (ROM); random access memory (RAM); magnetic disk storage media; optical storage media; flash memory devices; electrical, optical, acoustic, or other forms of propagated signals (e.g., carrier waves, infrared signals, digital signals, etc.), and other. Further, firmware, software, routines, and/or instructions may be described herein as performing certain actions. However, it should be appreciated that such descriptions are merely for convenience and that such actions in fact result from computing devices, processors, controllers, or other devices executing the firmware, software, routines, instructions, etc.

The term “analyte” as used herein may refer to a substance or chemical constituent of interest for pattern analysis. The analyte may include glucose. The analyte may include ketones. The analyte may include lactate. The analyte may include alcohol. The analyte may include one or more metabolic analytes, for example but not limited to, glucose, ketones, lactate, lactic acid, oxygen, hemoglobin A1C, lactone, lactose, galactose, vitamin C, glucoronate, glycogen, mannose, phosphate, bisphosphate, fructose, glyceraldehyde, glycerol, triglycerides, sorbitol, phosphoglucono, phosphogluconate, xylulose, ribose, bile, cysteine, serine, homoserine, pyruvate, phenylpyruvate, glutamate, glycine, taurine, threonine, methionine, ethanol, acetone, acetate, oxaloacetate, alanine, phenylalanine, aspartate, asparagine, alcohol, cholesterol, vitamin D, progesterone, testosterone, estrogen, squalene, insulin, hydroxybutyrate, leucine, isoleucine, malonyl, malonate, glucagon, epinephrine, norepinephrine, palmitate, lysine, eicosanoids, melanin, dopamine, tyrosine, tryptophan, niacin, melatonin, serotonin, citrate, isocitrate, valine, porphyrins, histidine, urocanate, histamine, glutamine, proline, creatine, putrescine, spermidine, spermine, arginine, ornithine, citrulline, fumarate, succinate, argininosuccinate, succinyl, ketoglutarate, aconitate, glyoxylate, caffeine, sugars, or carbs.

The term “analyte profile” as used herein may refer to a segment of analyte data of a user. In some aspects, an analyte profile may represent a segment of analyte data (e.g., glucose data) over less than a 24-hour time window. For example, the analyte profile may represent an N-hour time window, where N is an integer from 1 to 23 (e.g., 3-hour time window, 6-hour time window, 12-hour time window, etc.). In some aspects, an analyte profile may represent a segment of analyte data (e.g., glucose data) over a specific time window. For example, the analyte profile may represent a breakfast time window (e.g., 6:00 to 10:00), a lunch time window (e.g., 11:00 to 14:00), a dinner time window (e.g., 17:00 to 22:00), or a snack time window (e.g., 14:00 to 17:00). In some aspects, the analyte profile may be a glucose profile. In some aspects, the analyte profile may be a ketones profile. In some aspects, the analyte profile may be a lactate profile. In some aspects, an analyte profile may represent a segment of analyte data (e.g., glucose data) over a 24-hour time window. For example, the analyte profile may be a daily analyte profile. In some aspects, the analyte profile may be a daily glucose profile. In some aspects, the analyte profile may be a daily ketones profile. In some aspects, the analyte profile may be a daily lactate profile.

The term “distance matrix” as used herein may refer to a square matrix (2D array) containing the distances, taken pairwise, between the elements of a set. In some aspects, the distance matrix is an N×N symmetric matrix representing the distance between each pair of N daily glucose profiles. In some aspects, the distance may be defined by a metric. For example, the metric may include one or more of a mean absolute difference (MAD), a median absolute difference, a weighted mean absolute difference (WMAD), a weighted median absolute difference, a mean absolute relative difference (MARD), a median absolute relative difference, a precision absolute relative difference (PARD), an absolute difference, an absolute deviation, or a standard deviation. For example, the metric may be the MARD, which is a common and simple statistical approach to evaluate measurement accuracy and performance of the analyte sensor (e.g., CGM) as a single numeric value. Further, MARD is quick to calculate, may be compared between different CGM systems, reflects glycemic variability, and is unaffected by certain factors (e.g., physiological factors, pathological factors, etc.).

The term “point set” as used herein may refer to a set of points in a plane. In some aspects, each point of the point set may represent an analyte profile. For example, each analyte profile may be represented as a point on a graph, the distance between two points reflecting the distance between the two corresponding analyte profiles, allowing the difference (e.g., comparison) between two analyte profiles to be seen graphically. In some aspects, the point set may be represented as a scatter plot using Cartesian coordinates (X-axis, Y-axis) to display the collection of points at corresponding horizontal and vertical positions. In some aspects, the Euclidean distance between two points of the point set is the distance between the two corresponding analyte profiles (e.g., daily glucose profiles).

The term “multidimensional scaling” as used herein may refer to a process to translate elements of the distance matrix to the point set in a plane such that a distance between two points of the point set is the distance between a corresponding pair of the analyte profiles (e.g., daily glucose profiles). In some aspects, given a distance matrix with the distances between each pair of analyte profiles (e.g., daily glucose profiles), the multidimensional scaling may include an algorithm to place each analyte profile (e.g., daily glucose profile) into an N-dimensional space (e.g., N=1, 2, 3, etc.) such that the distances between the analyte profiles is preserved. In some aspects, the multidimensional scaling algorithm may include classical multidimensional scaling (e.g., principal coordinates analysis), metric multidimensional scaling (mMDS), non-metric multidimensional scaling (NMDS), or generalized multidimensional scaling (GMDS). For example, principal coordinates analysis (PCoA) may be used to minimize a loss function (e.g., a strain) based on dissimilarities between pairs of elements, and provides visual inspection and exploration to analyze the structure visually. For example, mMDS may provide a visual representation between complex data, reduce dimensionality for easier interpretation, and preserve distance to maintain the data's structure.

The term “cluster analysis” as used herein may refer to a process of grouping the point set (e.g., representing each of the daily glucose profiles) into two or more patterns such that daily glucose profiles of each pattern are more similar (e.g., as defined by a threshold or a metric) to each other than to those in other patterns. In some aspects, the cluster analysis may include one or more algorithms to separate the point set into two or more patterns. For example, the one or more algorithms may include a centroid model (e.g., k-means clustering), a model-based clustering (e.g., a Gaussian mixture model), a connectivity model (e.g., hierarchical clustering), or a self-organizing mapping (e.g., unsupervised neural network). For example, the algorithm may include k-means clustering that may provide efficient computation for real-time analysis, scalability for small or large datasets, and flexibility for utilizing different distance metrics. For example, the algorithm may include a Gaussian mixture model that may provide speed for fitting datasets quickly, robustness to accommodate outliers, a probabilistic approach for soft clustering and assigning different probabilities to different clusters, and flexibility to handle a wide range of complex distributions. In some aspects, the cluster analysis may utilize a metric to identify (e.g., classify) the two or more patterns from each other. For example, the metric may include a distance reduction ratio, a Dunn index, a Davies-Bouldin index, a silhouette coefficient, a purity, a Rand index, an F-measure, a Jaccard index, a Dice index, a Fowlkes-Mallows index, a Chi index, a confusion matrix, a Hopkins statistic. In some aspects, the metric may include a threshold above which the two or more patterns are identified (e.g., classified).

The term “smart device” as used herein may refer to an electronic device generally connected to one or more other devices or networks via different wireless protocols. In some aspects, the smart device may be capable of performing autonomous computing operations and connecting to other devices. In some aspects, the smart device may include a smart phone, a smart tablet, smart glasses, a smart keychain, a smart refrigerator, a smart car, a smart watch, a smart strap (e.g., Apple Watch, Fitbit Inspire, Biostrap EVO, Whoop, etc.), or a smart ring (e.g., Oura Ring, etc.).

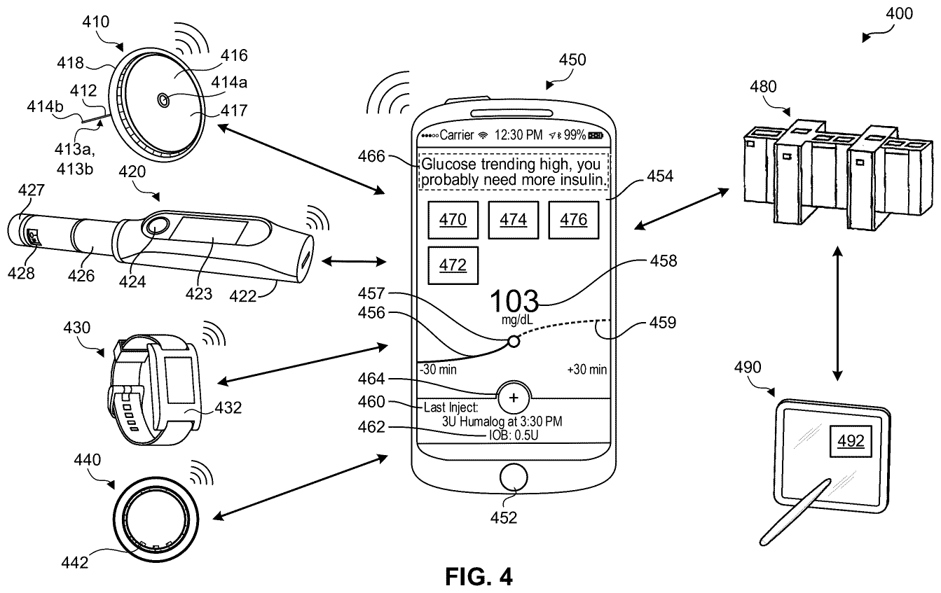

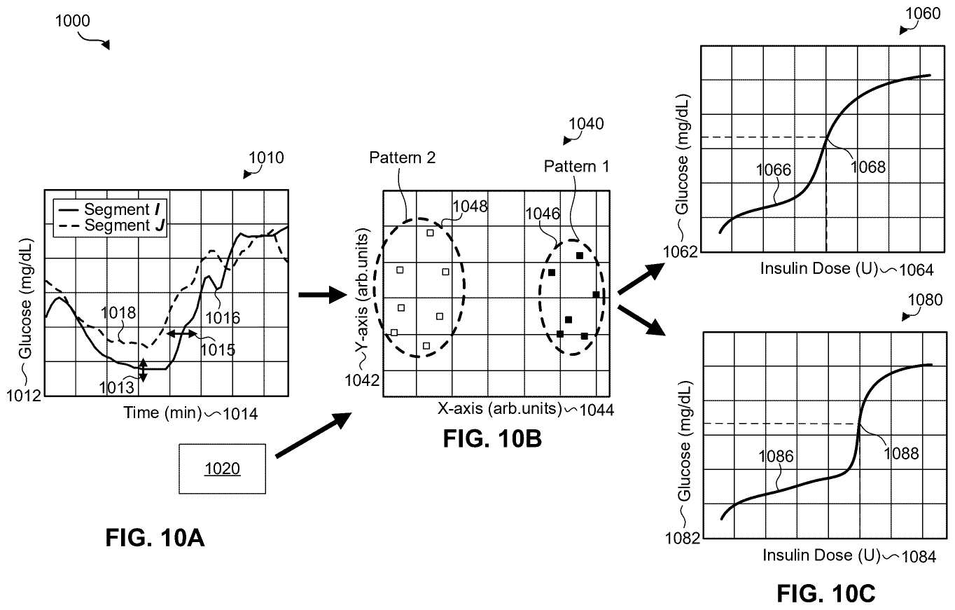

The term “titration profile” as used herein may refer to a graph or curve of glucose level (mg/dL) as a function of dose amount (U), for example, insulin dose amount (U). For example, as shown in FIG. 10C, titration profile 1066 may represent the dependence of glucose level (mg/dL) on insulin dose amount (U) (e.g., basal insulin dose, meal bolus dose, correction dose) to properly titrate to an optimal value (e.g., equivalence point 1068), for example, a glucose level of 100 mg/dL. If the glucose level (mg/dL) is below the optimal value (e.g., 100 mg/dL), a lower insulin dose (U) may be recommended, whereas if the glucose level (mg/dL) is above the optimal value (e.g., 100 mg/dL), a higher insulin dose (U) may be recommended. In some aspects, the titration profile may be based on one or more parameters including insulin dose amount, insulin sensitivity factor (ISF), and carbohydrate-to-insulin ratio (CR). In some aspects, the titration profile may be further discretized or applied to generate titration sub-profiles, for example, a meal titration sub-profile (e.g., a breakfast profile, a lunch profile, a dinner profile, a weekday breakfast profile, a weekday lunch profile, a weekday dinner profile, a weekend breakfast profile, a weekend lunch profile, a weekend dinner profile), a correction dose titration sub-profile, a basal dose titration sub-profile, etc. In some aspects, the titration profile may be used to generate one or more dose recommendations, for example, using the glucose data and one or more parameters of insulin dose amount, ISF, and/or CR of the titration profile to provide fixed dosed recommendations to a user (e.g., breakfast dose, lunch dose, dinner dose, etc.). In some examples, a titration profile may include a total daily dose (TDD) amount, which can be the same or different between titration profiles. In some aspects, the titration profile may be used with a medication delivery pump (e.g., an insulin pump). For example, the titration profile may be used to update a basal rate for an insulin pump. In some aspects, the titration profile may be used with a medication delivery device (e.g., an insulin injection pen, smart insulin pen), including a smart cap (e.g., a smart insulin pen cap). For example, the titration profile may be used to update a dose regimen (e.g., breakfast dose, lunch dose, dinner dose, etc.) on an insulin pen or on a smart insulin cap (e.g., pen cap 422 shown in FIG. 4).

Before describing such aspects in more detail, however, it is instructive to present example environments in which aspects of the present disclosure may be implemented.

### Example Analyte Monitoring System

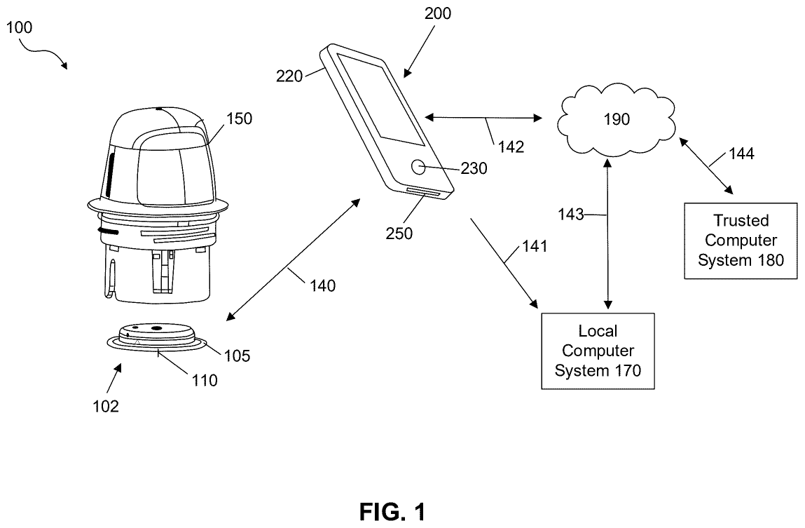

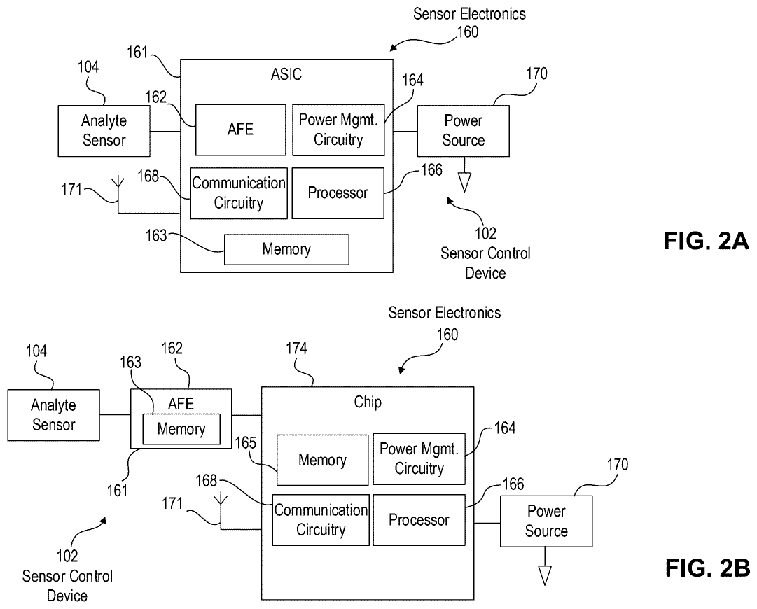

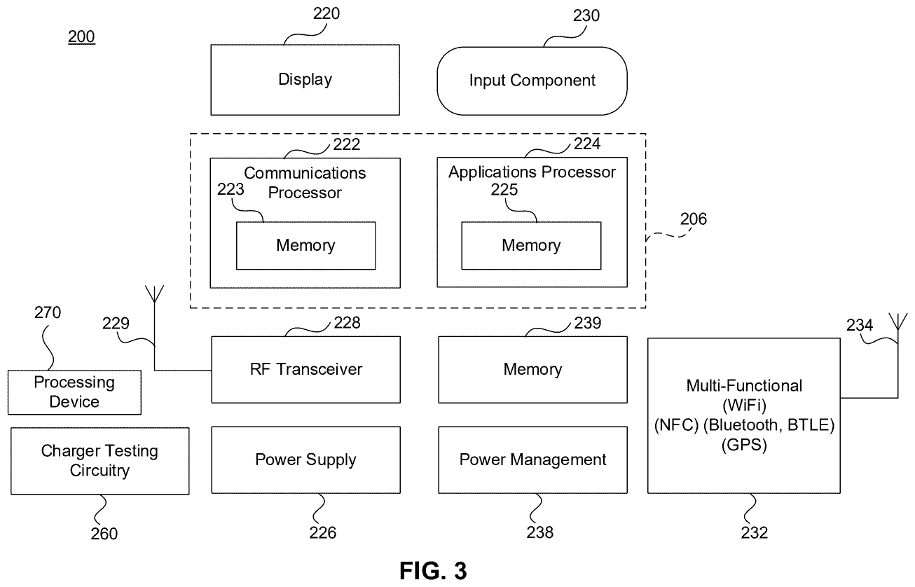

FIG. 1 is a conceptual diagram depicting an example aspect of an analyte monitoring system 100 that includes a sensor applicator 150, a sensor control device 102, and a receiver device 200. Sensor applicator 150 may be used to deliver sensor control device 102 to a monitoring location on a user's skin where an in vivo analyte sensor 104 is maintained in position for a period of time by an adhesive patch 105. Sensor control device 102 is further described in FIGS. 2A and 2B, and may communicate with receiver device 200 via a first communication path 140 using a wired or wireless technique. Example wireless protocols include Bluetooth, Bluetooth Low Energy (BLE, BTLE, Bluetooth SMART, etc.), Near Field Communication (NFC), and others. Users may view and use applications installed in memory on receiver device 200 using display 220 (which, in some aspects, may include a touchscreen), and input component 230 arranged on housing 210. A power source of receiver device 200 may be recharged using charging port 250. While only one receiver device 200 is shown, sensor control device 102 may communicate with multiple receiver devices 200. Each of the receiver devices 200 may communicate and share data with one another. More details about receiver device 200 are set forth with respect to FIG. 3 below.

Receiver device 200 may communicate with local computer system 170 via a second communication path 141 using a wired or wireless communication protocol. Local computer system 170 may include one or more of a laptop, desktop, tablet, smartphone, set-top box, video game console, or other computing device, and wireless communication may include any of a number of applicable wireless networking protocols including Bluetooth, Bluetooth Low Energy (BTLE), Wi-Fi, or others. Local computer system 170 may communicate via fourth communications path 143 with a network 190, similar to how receiver device 200 may communicate via a third communications path 142 with network 190, by a wired or wireless communication protocol as described previously. Network 190 may be any of a number of networks, such as private networks and public networks, local area or wide area networks, and so forth. A trusted computer system 180 may include one or more servers and may provide authentication services and secured data storage, and may communicate via fifth communications path 144 with network 190 by wired or wireless techniques.

FIGS. 2A and 2B are block diagrams depicting example aspects of sensor control devices 102 having in vivo analyte sensors 104 and sensor electronics 160 (including analyte monitoring circuitry) that may have the majority of the processing capability for rendering end-result data suitable for display to the user. In FIG. 2A, a single semiconductor chip 161 is depicted that may be a custom application specific integrated circuit (ASIC). Shown within ASIC 161 are certain high-level functional units, including an analog front end (AFE) 162, power management (or control) circuitry 164, processor 166, and communication circuitry 168 (which may be implemented as a transmitter, receiver, transceiver, passive circuit, or otherwise according to the communication protocol). In this aspect, both AFE 162 and processor 166 are used as analyte monitoring circuitry, but in other aspects either circuit may perform the analyte monitoring function. Processor 166 may include one or more processors, microprocessors, controllers, and/or microcontrollers, each of which may be a discrete chip or distributed amongst (and a portion of) a number of different chips.

A memory 163 is also included within ASIC 161 and may be shared by the various functional units present within ASIC 161, or may be distributed amongst two or more of them. Memory 163 may also be a separate chip. Memory 163 may be volatile and/or non-volatile memory. In this aspect, ASIC 161 is coupled with power source 172, which may be a battery, or the like. AFE 162 interfaces with in vivo analyte sensor 104 and receives measurement data therefrom and outputs the data to processor 166 in digital form, which in turn processes the data to arrive at the end-result analyte data, such as discrete analyte levels or trend values, etc. This data may then be provided to communication circuitry 168 for sending, by way of antenna 171, to receiver device 200 or other devices, for example, where minimal further processing is needed by the resident software application to display the data.

FIG. 2B is similar to FIG. 2A but instead includes two discrete semiconductor chips 161 and 174, which may be packaged together or separately. Here, AFE 162 is resident on ASIC 161. Processor 166 is integrated with power management circuitry 164 and communication circuitry 168 on chip 174. AFE 162 includes memory 163 and chip 174 includes memory 165, which may be isolated or distributed within. In one example aspect, AFE 162 is combined with power management circuitry 164 and processor 166 on one chip, while communication circuitry 168 is on a separate chip. In another example aspect, both AFE 162 and communication circuitry 168 are on one chip, and processor 166 and power management circuitry 164 are on another chip. It should be noted that other chip combinations are possible, including three or more chips, each bearing responsibility for the separate functions described, or sharing one or more functions for fail-safe redundancy.

FIG. 3 is a block diagram depicting an example aspect of a receiver device 200. Receiver device 200 may include a housing having a display 220 and one or more input components 230, such as for receiving user input. Receiver device 200 may include one or more processors, and the processors may be coupled to memory. Receiver device 200 may include a processing core 206, including a communications processor 222 coupled with first memory 223 and an applications processor 224 coupled with second memory 225 as shown in FIG. 3. Also included may be separate memory 239. Receiver device 200 may include communication circuitry for receiving analyte data from sensor control device 102, or for communicating with other components of analyte monitoring system 100, such as local computer system 170, trusted computer system 180, or network 190. The communication circuitry may include a transceiver, such as a RF transceiver 228 with antenna 229, and/or a multi-functional transceiver 232 which may communicate over one or more of Wi-Fi, NFC, Bluetooth, BTLE, and GPS with an antenna 234. Receiver device 200 may include a power storage device 226, and may further include a power management module 238. A charging port for charging power storage device 226 may be in electrical communication with power storage device 226 and/or power management module 238. Charger testing circuitry 260 may be in electrical communication with power storage device 226 and/or power management module 238. As understood by one of skill in the art, these components are electrically and communicatively coupled in a manner to make a functional device.

Receiver device 200 may be configured to detect whether a connected charging device is suitable for use with receiver device 200. A suitable charging device either has a power that is at or below the power requirement for the receiver device, or has a protection mechanism against short circuit. In some aspects, the power output from the charging device should be no more than a maximum power, such as 3 W. In some aspects, the maximum power may be in a range of 2.5 W to 3 W for a suitable charging device.

Instructions for the charging device testing process may be stored in a memory of receiver device, e.g., one or more of memory 223, 225, 239. When executed by a processing device, receiver device 200 is configured to perform a series of steps to detect whether a connected charging device is suitable for use with the receiver device and to alert the user if not. A processing device 270 in communication with charger testing circuitry 260, as shown for example in FIG. 3, may determine a voltage of charger testing circuitry 260 after a test charge is provided to a connected charging device. Processing device 270 may determine voltage of charger testing circuitry 260 at different locations within charger testing circuitry 260, as further described herein. Processing device 270 may be a microcontroller, and may include an analog-to-digital converter. Processing device 270 may be a comparator, among other devices. The use of a separate, low power device, such as a comparator as processing device 270 may help to conserve power relative to using the processors 222, 224 of receiver device 200, as processors 222, 224 of receiver device 200 may have greater power consumption. Alternatively, instead of processing device 270, one or both of processors 222 or 224 may determine a voltage of charger testing circuitry 260, which may help to reduce the number of components and to simplify construction and manufacturing of receiver device 200.

Receiver device 200 is configured to communicate with a sensor control device 102. Sensor control device 102 may include an in vivo analyte sensor 104, such as an in vivo glucose sensor, as described herein. However, in other aspects, one or more analytes may be measured by the in vivo analyte sensor, such as one or more of glucose, ketones, lactate, or alcohol. Other analytes that may be monitored with system 100 include, but are not limited to, acetyl choline, amylase, bilirubin, cholesterol, chorionic gonadotropin, glycosylated hemoglobin (HbAlc), creatine kinase (e.g., CK-MB), creatine, creatinine, DNA, fructosamine, glucose, glucose derivatives, glutamine, growth hormones, hormones, ketones, ketone bodies, lactate, peroxide, prostate-specific antigen, prothrombin, RNA, thyroid stimulating hormone, and troponin. The concentration of drugs, such as, for example, antibiotics (e.g., gentamicin, vancomycin, and the like), digitoxin, digoxin, drugs of abuse, theophylline, and warfarin, may also be monitored. When monitoring more than one analyte, the analytes may be monitored at the same or different times.

Receiver device 200 may receive data from sensor control device 102, such as raw or processed analyte data. Receiver device 200 may determine and/or display analyte metrics based on the received analyte data. Receiver device 200 may use artificial intelligence, such as machine learning, e.g., deep learning, to analyze the received analyte data. Artificial intelligence may be used to detect patterns in the user's analyte data, predict future glucose levels, predict extreme glucose events such as episodes of hypoglycemia or hyperglycemia, identify lifestyle events such as meals, medication dose administration, or exercise, or to generate recommendations to improve control of glucose levels, among other functions.

Display 220 of receiver device 200 may display analyte data and other information. Display 220 may present a graphical user interface (GUI). Receiver device 200 may display at least a portion of the analyte data received from the in vivo analyte sensor. Receiver device 200 may display a plot of analyte levels over time based on received analyte data. The plot of analyte levels may be updated in real time as analyte data is received by receiver device 200. Receiver device 200 may display a current analyte level, and/or an analyte trend level. Receiver device 200 may output alerts. Alerts may correspond to an analyte level condition, such as a high glucose level, a low glucose level, or a very low glucose level, among others. The alerts may be based on one or more analyte level thresholds, which may be predetermined or adjustably set by the user or by a healthcare professional. Alerts may also correspond to system conditions, such as a temperature of the sensor, a communication error, a battery error, or an error related to sensor accuracy, among others.

### Example Analyte Pattern Analysis System

Existing daily monitoring schemes (e.g., AGP) may be difficult to interpret by patients and even by the HCP. Such methods fail to analyze day-to-day pattern variations caused by changing daily habits and routines of individuals. Further, existing reports of daily monitoring schemes (e.g., AGP reports) neither identify multiple distinct recurring patterns nor present identified patterns in a clear, visually distinguishable way to provide insights and assist a user or health care professional better understand the user's multi-day analyte data (e.g., glucose data) and correlated factors. Moreover, current daily monitoring schemes do not use machine learning to streamline day-to-day analyte pattern analysis and provide configurable pattern identification metrics.

Aspects of analyte pattern analysis apparatuses, systems, and methods as discussed herein may identify and visualize distinct patterns of analyte data (e.g., glucose data). Aspects of analyte pattern analysis apparatuses, systems, and methods as discussed herein may identify recurring analyte patterns (e.g., recurring glucose patterns). Aspects of analyte pattern analysis apparatuses, systems, and methods as discussed herein may identify correlations between analyte patterns and external events (e.g., medication dosing, meals, exercise, sleep, stress, etc.). Aspects of analyte pattern analysis apparatuses, systems, and methods as discussed herein may help reduce glycemic variability. Aspects of analyte pattern analysis apparatuses, systems, and methods as discussed herein may output a report identifying and visually distinguishing two or more patterns associated with an individual's multi-day analyte data (e.g., multi-day glucose data). Aspects of analyte pattern analysis apparatuses, systems, and methods as discussed herein may streamline pattern analysis with machine learning methods (e.g., unsupervised machine learning). Aspects of analyte pattern analysis apparatuses, systems, and methods as discussed herein may provide one or more recommendations for user intervention or user action based on identified patterns. Aspects of analyte pattern analysis apparatuses, systems, and methods as discussed herein may develop personalized and optimized treatment for an individual.

In some aspects, an analyte pattern analysis system may include an on-body unit (OBU) with an analyte sensor, a receiver device, and a software application. The processing of the software application may be performed in part on the on-body unit (e.g., OBU 410), on the receiver device (e.g., receiver device 450), or remotely (e.g., on remote server 480). The analyte sensor may be configured to monitor one or more analyte levels of a user (e.g., glucose levels). The receiver device may be configured to receive therapy data of the user (e.g., glucose data, medication dosing data, meal data, activity data, etc.). The software application may be configured to analyze analyte profiles (e.g., daily glucose profiles) of the therapy data over an analysis period. The analysis may be performed by a machine learning model (e.g., unsupervised machine learning). The analysis may include grouping the analyte profiles (e.g., the daily glucose profiles) into two or more patterns based on the analysis of the machine learning model. The software application may be configured to output a report including identification of the two or more patterns associated with each analyte profile (e.g., daily glucose profile). Advantageously, the analyte pattern analysis system may identify and visualize distinct patterns of therapy data (e.g., glucose data). Further advantageously, the analyte pattern analysis system may identify recurring analyte patterns (e.g., glucose patterns). Further advantageously, the analyte pattern analysis system may identify correlations between analyte patterns and external events (e.g., medication dosing, meals, exercise, sleep, stress, etc.). Further advantageously, the analyte pattern analysis system may streamline pattern analysis with the machine learning model. Further advantageously, the analyte pattern analysis system may identify and visually distinguish two or more patterns associated with an individual's multi-day analyte data (e.g., glucose data) in the report. Further advantageously, the analyte pattern analysis system may provide one or more recommendations for user intervention or user action based on the identified patterns. Further advantageously, the analyte pattern analysis system may develop personalized and optimized treatment for an individual.

FIG. 4 illustrates analyte pattern analysis system 400 with software application 470, according to example aspects. Analyte pattern analysis system 400 may be configured to identify and visualize distinct patterns of analyte data, identify recurring analyte patterns, and reduce analyte variability of an individual. Analyte pattern analysis system 400 may be further configured to streamline pattern analysis with a machine learning model (e.g., unsupervised machine learning). Analyte pattern analysis system 400 may be further configured to output a report identifying and visually distinguishing two or more patterns associated with an individual's multi-day analyte data, and provide one or more recommendations for user intervention or user action based on identified patterns. Analyte pattern analysis system 400 may be further configured to identify correlations between analyte patterns and external events (e.g., medication dosing, meals, exercise, sleep, stress, etc.), and develop personalized and optimized treatment for an individual.

Although analyte pattern analysis system 400 is shown in FIG. 4 as a stand-alone apparatus and/or system, aspects of this disclosure may be used with other apparatuses, systems, and/or methods, such as, but not limited to, elements in FIGS. 1-3 and 5-9, e.g., analyte monitoring system 100, sensor control device 102, receiver device 200, flow diagram 500, machine learning model 600, cluster plot 700, calendar plot 800, and/or distance plot 900.

As shown in FIG. 4, analyte pattern analysis system 400 may include on-body unit (OBU) 410, also referred to as an analyte monitoring device, or when glucose is the analyte a “glucose monitoring device.” In some aspects, analyte pattern analysis system 400 may omit medication delivery device 420, first smart device 430, and/or second smart device 440, for example, when only analyzing analyte data of a user rather than also considering therapy data of the user (e.g., medication dosing data, meal data, activity data, etc.). In some aspects, analyte pattern analysis system 400 may utilize just OBU 410, receiver device 450, and software application 470 to analyze daily analyte profiles and identify recurring analyte patterns.

As described herein, sensor control device 102 shown in FIGS. 1 and 2A-2B and OBU 410 shown in FIG. 4 represent the same device for measuring and communicating sensor data of an analyte (e.g., glucose) of a user. Also, sensor control device 102 shown in FIGS. 1 and 2A-2B and OBU 410 shown in FIG. 4 are referred to herein as an analyte monitoring device, or when glucose is the analyte, as a glucose monitoring device.

OBU 410 may be configured to measure and communicate sensor data of an analyte (e.g., glucose) of a user. OBU 410 may be further configured to communicate therapy data (e.g., glucose data) from analyte sensor 412 to one or more components of analyte pattern analysis system 400 (e.g., receiver device 450, software application 470, remote server 480, etc.). As shown in FIG. 4, OBU 410 may include analyte sensor 412, sensor electronics 416, housing 417, and/or adhesive layer 418. In some aspects, analyte data may include daily analyte profiles over an analysis period. In some aspects, analyte data may include a time stamp (e.g., date and time) and a analyte value (e.g., glucose level mg/dL) for each time stamp.

Analyte sensor 412 may be configured to measure an analyte (e.g., glucose) of a user. In some aspects, analyte sensor 412 may be a continuous glucose monitor (CGM) sensor configured to monitor glucose levels of a user. As shown in FIG. 4, analyte sensor 412 may include a first portion 414a configured to be arranged above a skin surface of the user, and a second portion 414b configured to be arrange below the skin surface of the user and in contact with interstitial fluid of the user. In some aspects, analyte sensor 412 may include a single analyte sensor to measure first analyte 413a (e.g., a CGM sensor). In some aspects, first analyte 413a may be glucose.

In some aspects, analyte sensor 412 may be configured to measure one or more analytes (e.g., glucose, ketones, lactic acid, etc.) of a user. Analyte sensor 412 may be further configured to continuously measure (e.g., in vivo) in real-time a concentration of one or more analytes (e.g., first analyte 413a, second analyte 413b, etc.) of a user. As shown in FIG. 4, analyte sensor 412 may detect first analyte 413a (e.g., glucose) and/or second analyte 413b (e.g., ketones). In some aspects, a portion of analyte sensor 412 (e.g., second portion 414b) may be positioned in vivo through a skin surface of a user (e.g., transcutaneously) and in fluid contact with bodily fluids (e.g., blood, interstitial fluid, etc.) of the user. In some aspects, analyte sensor 412 may be insertable into a body of a patient (e.g., vein, artery, skin, etc.) containing an analyte. In some aspects, analyte sensor 412 may include CGM to continuously and automatically track glucose levels of the user.

In some aspects, for example, as shown in FIG. 4, analyte sensor 412 may include a single (dual) analyte sensor to measure first and second analytes 413a, 413b (e.g., simultaneously). In some aspects, analyte sensor 412 may include two separate analyte sensors to measure first and second analytes 413a, 413b (e.g., a CGM sensor and a separate continuous ketone monitoring sensor). In some aspects, first analyte 413a may be glucose and second analyte 413b may be ketones.

In some aspects, analyte sensor 412 may automatically and/or continuously monitor one or more analyte levels (e.g., first analyte 413a, second analyte 413b, etc.) in vivo, for example, glucose and ketones of a patient, over a predetermined time interval (e.g., sensor lifetime) or given sensing period (e.g., 7 days, 14 days, 15 days, 28 days, or 30 days, etc.). In some aspects, analyte sensor 412 may be coupled (e.g., electronically) to sensor electronics 416 to process information obtained from analyte sensor 412 (e.g., sensor data). In some aspects, analyte sensor 412 may be in communication (e.g., wired, wirelessly) with sensor electronics 416.

In some aspects, analyte sensor 412 may measure glucose. In some aspects, analyte sensor 412 may measure one or more analytes. For example, analyte sensor 412 may measure one or more metabolic analytes, for example but not limited to, glucose, ketones, lactate, lactic acid, oxygen, hemoglobin A1C, lactone, lactose, galactose, vitamin C, glucoronate, glycogen, mannose, phosphate, bisphosphate, fructose, glyceraldehyde, glycerol, triglycerides, sorbitol, phosphoglucono, phosphogluconate, xylulose, ribose, bile, cysteine, serine, homoserine, pyruvate, phenylpyruvate, glutamate, glycine, taurine, threonine, methionine, ethanol, acetone, acetate, oxaloacetate, alanine, phenylalanine, aspartate, asparagine, alcohol, cholesterol, vitamin D, progesterone, testosterone, estrogen, squalene, insulin, hydroxybutyrate, leucine, isoleucine, malonyl, malonate, glucagon, epinephrine, norepinephrine, palmitate, lysine, eicosanoids, melanin, dopamine, tyrosine, tryptophan, niacin, melatonin, serotonin, citrate, isocitrate, valine, porphyrins, histidine, urocanate, histamine, glutamine, proline, creatine, putrescine, spermidine, spermine, arginine, ornithine, citrulline, fumarate, succinate, argininosuccinate, succinyl, ketoglutarate, aconitate, glyoxylate, caffeine, sugars, or carbs, etc. In some aspects, analyte sensor 412 may measure one or more analytes simultaneously with one or more corresponding electrochemical biosensors for each different analyte measured.

Sensor electronics 416 may be configured to process signals from analyte sensor 412. Sensor electronics 416 may be further configured to communicate data (e.g., glucose data) from analyte sensor 412 to one or more external devices (e.g., receiver device 450, software application 470, remote server 480, etc.). Sensor electronics 416 may be further configured to wirelessly communicate (e.g., NFC, WiFi, Bluetooth, BLE, Internet, etc.) analyte related sensor data (e.g., glucose data). As shown in FIG. 4, sensor electronics 416 may be operatively (e.g., electrically) coupled to analyte sensor 412 and wirelessly coupled to receiver device 450 and/or software application 470.

In some aspects, sensor electronics 416 may include a printed circuit board (PCB) for connection to various components (e.g., analyte sensor 412, processor, ASIC, wireless transceiver, wireless transmitter, controller, memory, etc.). In some aspects, sensor electronics 416 may store (e.g., via memory) historical analyte related data (e.g., daily glucose data). In some aspects, sensor electronics 416 may be configured to store some or all of analyte related data (e.g., glucose data) from analyte sensor 412 in a memory, for example, during an analysis period (e.g., 7 days, 14 days, 15 days, 28 days, or 30 days, etc.). In some aspects, sensor electronics 416 may include one or more processors and/or control logic configured to determine (e.g., via software programs and/or algorithms) current analyte levels (e.g., glucose levels), rates of change (ROC) of analyte levels (e.g., glucose ROC), rates of acceleration of analyte levels (e.g., rate of glucose ROC), analyte trend information (e.g., graph 456), and/or analyte fluctuation levels (e.g., standard deviation, etc.).

Housing 417 may be configured to provide an interior compartment for a portion of analyte sensor 412 (e.g., proximal portion) and sensor electronics 416. As shown in FIG. 4, housing 417 may include analyte sensor 412 and sensor electronics 416, and be coupled to adhesive layer 418. In some aspects, housing 417 may include a sealed housing (e.g., hermetically sealed biocompatible housing). Adhesive layer 418 may be configured to attach OBU 410 to a skin surface of a user. As shown in FIG. 4, adhesive layer 418 may be coupled to housing 417 to securely position a portion of analyte sensor 412 (e.g., second portion 414b) below a skin surface of a user.

As shown in FIG. 4, analyte pattern analysis system 400 may include medication delivery device 420. Medication delivery device 420 may be configured to administer and monitor medication doses to a user. Medication delivery device 420 may be further configured to communicate therapy data (e.g., medication dosing data) to one or more components of analyte pattern analysis system 400 (e.g., receiver device 450, software application 470, remote server 480, etc.). In some aspects, medication dosing data may include daily medication dosing events over an analysis period. In some aspects, medication dosing data may include a dosing type (e.g., basal insulin, prandial insulin, pre-mixed insulin, GLP-1, etc.), a dosing time stamp (e.g., date and time), a dosing time range (e.g., time since last dose), and/or a dose amount (e.g., 5 U) for each dosing time stamp. As shown in FIG. 4, medication delivery device 420 may include pen cap 422 and injection pen 426. In some aspects, medication delivery device 420 may include a smart insulin cap where the functionality of pen cap 422 described below is embedded in injection pen 426. In some aspects, medication delivery device 420 may include a smart injection pen where the functionality of pen cap 422 described below is embedded in injection pen 426.

Pen cap 422 may be configured to monitor medication dosing data. Pen cap 422 may be further configured to detect a dosing event of injection pen 426 (e.g., insulin pen, GLP-1 pen, etc.). For example, the dosing event may be inferred from a decapping event of pen cap 422 from injection pen 426 and a capping event of pen cap 422 to injection pen 426. As shown in FIG. 4, pen cap 422 may include display 423 and input button 424. Display 423 may be configured to provide information and/or notifications to the user. Input button 424 may be configured to provide a control input for the user to input information (e.g., type of injection pen, dose amount, recommended dose amount, etc.).

Injection pen 426 may be configured to administer medication doses to a user. As shown in FIG. 4, injection pen 426 may include dial 427 and dose indicator 428. Dial 427 may be configured to set a dosage to be delivered and dose indicator 428 may be configured to indicate the dose amount. In some aspects, injection pen 426 may include a manual injection pen. For example, the manual injection pen may include insulin (e.g., long-acting (LA) insulin, rapid-acting (RA) or prandial insulin), glucagon-like peptide-1 (GLP-1) receptor agonists, or dual gastric inhibitory peptide (GIP)/GLP-1 receptor agonists.

System 400 may include a first smart device 430 that is configured to monitor external events of a user (e.g., activity data). First smart device 430 may be further configured to communicate therapy data (e.g., activity data, meal data, exercise data, etc.) to one or more components of analyte pattern analysis system 400 (e.g., receiver device 450, software application 470, remote server 480, etc.). In some aspects, activity data may include daily activity events over an analysis period. In some aspects, activity data may include an activity event of the user (e.g., a meal event), an exercise event of the user, a stress event of the user, a sleep event of the user, a location event of the user, a travel event of the user, or a combination thereof. In some aspects, first smart device 430 may include a smart strap (e.g., Apple Watch, Fitbit Inspire, Biostrap EVO, Whoop, etc.). As shown in FIG. 4, first smart device 430 may include one or more sensors 432 (e.g., position sensor, motion sensor, activity sensor, etc.) configured to detect and measure activity data of the user.

System 400 may optionally include a second smart device 440 to monitor external events of a user (e.g., activity data). Second smart device 440 may be further configured to communicate therapy data (e.g., activity data, stress data, sleep data, etc.) to one or more components of analyte pattern analysis system 400 (e.g., receiver device 450, software application 470, remote server 480, etc.). In some aspects, activity data may include daily activity events over an analysis period. In some aspects, activity data may include an activity event of the user (e.g., a meal event), an exercise event of the user, a stress event of the user, a sleep event of the user, a location event of the user, a travel event of the user, or a combination thereof. In some aspects, second smart device 440 may include a smart ring (e.g., Oura Ring, etc.). As shown in FIG. 4, second smart device 440 may include one or more sensors 442 (e.g., position sensor, motion sensor, activity sensor, sleep sensor, etc.) configured to detect and measure activity data of the user.

As shown in FIG. 4, analyte pattern analysis system 400 may include receiver device 450. Receiver device 450 may be configured to receive or retrieve therapy data from OBU 410 (e.g., glucose data), medication delivery device 420 (e.g., medication dosing data), first smart device 430 (e.g., meal data), and/or second smart device 440 (e.g., sleep data). Receiver device 450 may be further configured to communicate therapy data (e.g., glucose data, medication dosing data, activity data, stress data, sleep data, etc.) to one or more components of analyte pattern analysis system 400 (e.g., software application 470, remote server 480, etc.). In some aspects, receiver device 450 may include a dedicated reader device, such as may be provided by the CGM manufacturer, a handheld computer (e.g., smartphone, cell phone, mobile phone, PDA, smart watch, etc.), personal computer, laptop computer, or any other portable communication device. As shown in FIG. 4, receiver device 450 may be operatively (e.g., wirelessly, NFC, BLE, etc.) coupled to OBU 410, medication delivery device 420, first smart device 430, second smart device 440, software application 470, and/or remote server 480. In some aspects, receiver device 450 may be configured to send therapy data (e.g., daily glucose profiles) to software application 470 for pattern analysis and outputting a report. As shown in FIG. 4, receiver device 450 may include input component 452, display 454, software application 470, report 472, meal logs 474 (e.g., mobile app), and/or activity logs 476 (e.g., mobile app).

Input component 452 may be configured to control operation of receiver device 450. Input component 452 may be further configured to input data and/or commands to receiver device 450. As shown in FIG. 4, input component 452 may interact with receiver device 450 to control operation of receiver device 450. In some aspects, input component 452 may include a button, an actuator, a switch, a job wheel, a touch screen, a microphone, a camera, a combination thereof, or a similar input element. For example, input component 452 may be a touch screen or touch sensitive element of display 454.

Display 454 may be configured to display a variety of information-some or all of which may be displayed at the same time or at different times. Display 454 may be further configured to output alarms, notifications (e.g., warnings, recommendations, guidance, etc.), prompts, first analyte 413a (e.g., glucose) levels, second analyte 413b (e.g., ketones) levels, information (e.g., information display 460), messages (e.g., message 466), reports (e.g., report 472), or a combination thereof, which may be visual, audio, tactile, or a combination thereof. As shown in FIG. 4, display 454 may include, but is not limited to, graph 456 (e.g., historical trend), indicator 457, current glucose value 458, predictor 459 (e.g., estimated trend), information display 460 (e.g., type of injection, dose amount, time of last dose), insulin on board (IOB) 462 (e.g., insulin remaining within user), bolus calculator 464, and/or message 466.

Meal logs 474 may be configured to track and correlate meal data of the user. In some aspects, meal logs 474 may detect a meal event of the user (e.g., breakfast, lunch, dinner, snack). In some aspects, meal data may include a meal type, a meal description, a meal classification, a meal size, a meal time, a meal date, a meal time stamp, a meal frequency, a meal glycemic response, or other similar meal relevant information. In some aspects, meal logs 474 may be part of a mobile app. In some aspects, meal logs 474 may be input by a user. For example, a user may input meal data into receiver device 450 via a mobile app. In some aspects, meal logs 474 may be detected by one or more smart devices (e.g., first smart device 430 and/or second smart device 440) and received by receiver device 450 in communication with the one or more smart devices. In some aspects, meal logs 474 may be operatively (e.g., wirelessly, NFC, BLE, electronically, etc.) coupled to software application 470 to provide meal data of a user. In some aspects, meal logs 474 (e.g., mobile app) may be part of software application 470.

Activity logs 476 may be configured to track and correlate activity data of the user. In some aspects, activity logs 476 may detect an activity event of the user (e.g., exercise event, stress event, sleep event, location event, travel event). In some aspects, activity data may include exercise data, stress data, sleep data, location data, travel data, calendar data, or other similar activity relevant information. In some aspects, activity logs 476 may be part of a mobile app (e.g., Fitbit app, Oura app, etc.). In some aspects, activity logs 476 may be input by a user. For example, a user may input activity data into receiver device 450 via a mobile app. In some aspects, activity logs 476 may be detected by one or more smart devices (e.g., first smart device 430 and/or second smart device 440) and received by receiver device 450 in communication with the one or more smart devices. In some aspects, activity logs 476 may be operatively (e.g., wirelessly, NFC, BLE, electronically, etc.) coupled to software application 470 to provide activity data of a user. In some aspects, activity logs 476 (e.g., mobile app) may be part of software application 470.

As shown in FIG. 4, analyte pattern analysis system 400 may include software application 470. System 400 may be configured to identify and visualize distinct patterns of analyte data (e.g., patterns in daily glucose profiles). Software application 470 may be configured to identify recurring analyte patterns via a model. Software application 470 may be further configured to identify correlations between analyte patterns and external events (e.g., medication dosing, meals, exercise, sleep, stress, etc.) via a model. Software application 470 may be further configured to help reduce glycemic variability via one or more recommendations for therapy adjustment. Software application 470 may be further configured to output a report (e.g., report 472) identifying and visually distinguishing two or more patterns associated with an individual's multi-day analyte data. Software application 470 may be further configured to streamline pattern analysis using a machine learning model (e.g., unsupervised machine learning). Software application 470 may be further configured to provide one or more recommendations for user intervention or user action based on identified patterns. Software application 470 may be further configured to develop personalized and optimized treatment for an individual.

As shown in FIG. 4, software application 470 may be operatively coupled to OBU 410, receiver device 450, remote server 480, and/or remote device 490. In some aspects, software application 470 may be performed by one or more processors (e.g., processor, controller, microprocessor, microcontroller, ASIC, etc.). In some aspects, software application 470 may include and/or be coupled to a memory storing instructions which, when executed by at least one processor, cause the processor to perform operations including receiving therapy data of a user, including analyte data monitored by OBU 410. In some aspects, the operations may further include analyzing analyte profiles (e.g., daily glucose profiles) of the analyte data over an analysis period by a machine learning model. In some aspects, the operations may further include grouping the analyte profiles into two or more patterns based on the analysis performed by the machine learning model. In some aspects, the operations may further include outputting report 472 including identification of the two or more patterns associated with each analyte profile.

In some aspects, software application 470 may include a mobile application (app). For example, software application 470 may be part of receiver device 450 (e.g., in a mobile app). In some aspects, software application 470 may all be contained in a mobile application (app). In some aspects, some or part of software application 470 may be contained in remote server 480 (e.g., web-server, cloud server, etc.) that supports software application 470. In some aspects, software application 470 may be part of receiver device 450. In some aspects, software application 470 may be part of remote device 490, for example, at a remote location (e.g., HCP, clinic, etc.). In some aspects, software application 470 may include a web application (app).

Software application 470 may analyze analyte profiles of analyte data of a user over an analysis period by a machine learning model. In some aspects, a daily analyte profile may include analyte data of the user over a 24-hour time window. In some aspects, an analyte profile may include analyte data of the user over less than a 24-hour time window. For example, the analyte profile may represent an N-hour time window, where N is an integer from 1 to 23 (e.g., 3-hour time window, 6-hour time window, 12-hour time window, etc.). In some aspects, an analyte profile may represent a segment of analyte data over a specific time of day window. For example, the analyte profile (e.g., glucose profile) may represent a breakfast time window (e.g., 6:00 to 10:00), a lunch time window (e.g., 11:00 to 14:00), a dinner time window (e.g., 17:00 to 22:00), or a snack time window (e.g., 14:00 to 17:00).

In some aspects, the analysis period of software application 470 may be 3 or more days, 5 or more days, 7 or more days, and may be in a range from 3 days to 14 days, 5 days to 12 days, or 7 days to 12 days. In some aspects, software application 470 may limit the identifying to no greater than three patterns. For example, for a shorter analysis period (e.g., 14 days or less), the identifying may be limited to no greater than three patterns since for daily analyte profiles software application 470 is only considering 14 data points (e.g., n=14, one for each day) and more than three patterns may not be statistically significant.

In some aspects, the analysis period of software application 470 may be at least 14 days. In some aspects, the analysis period of software application 470 may be in a range from 3 days to 28 days. In some aspects, the analysis period of software application 470 may be at least 28 days. In some aspects, the analysis period of software application 470 may be in a range from 30 days to 90 days. In some aspects, the analysis period of software application 470 may be at least 45 days. For example, for a longer analysis period (e.g., greater than 14 days, greater than 28 days, greater than 45 days, etc.), the identifying may include three or more patterns (e.g., three patterns, four patterns, five patterns, etc.) since software application 470 is considering a larger number of data points (e.g., n>14, one for each day) and may thereby identify more patterns from the larger data set that are statistically significant.

In some aspects, software application 470 may analyze analyte profiles by assessing a distance between the analyte profiles. For example, software application 470 may analyte daily glucose profiles by the distance between pairs of the daily glucose profiles. In some aspects, the distance may be based on a MAD (e.g., Equation 1 above), a median absolute difference, a WMAD (e.g., Equation 2 above), a weighted median absolute difference, a MARD (e.g., Equation 3 above), a median absolute relative difference, a precision absolute relative difference (PARD), an absolute difference, an absolute deviation, a standard deviation, or any other suitable distance metric. For example, the distance may based on the MARD. In some aspects, the distance may be an adjustable parameter. In some aspects, software application 470 may group first and second analyte profiles into a first pattern type when the distance is at or below a threshold. For example, the threshold may include a predetermined analyte value (e.g., glucose difference of 30 mg/dL). In some aspects, if the distance is greater than a predetermined maximum analyte value, the analyte profile may be deemed to be an outlier (e.g., glucose difference greater than 70 mg/dL).

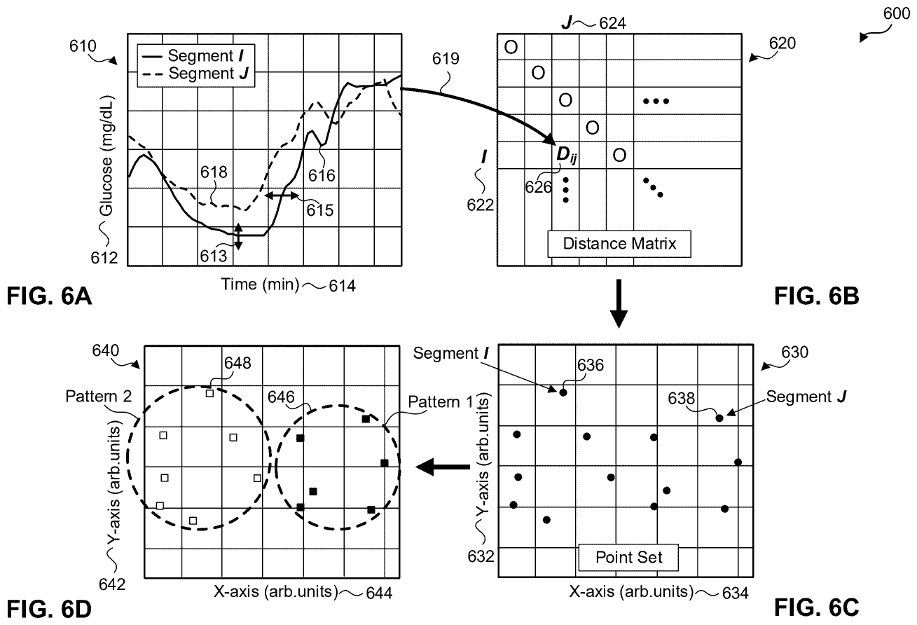

In some aspects, software application 470 may generate a distance matrix based on a distance between pairs of the analyte profiles. For example, as shown in FIGS. 6A and 6B, software application 470 may generate distance matrix 620 based on analysis (e.g., comparison) between daily glucose profiles. In some aspects, the distance matrix may be an N×N symmetric matrix representing the distance between each pair of N analyte profiles (e.g., daily glucose profiles). In some aspects, the distance may be based on the MARD, which is a simple statistical approach to evaluate measurement accuracy and performance of the analyte sensor (e.g., CGM) as a single numeric value, reflects analyte variability (e.g., glycemic variability), and is unaffected by certain factors (e.g., physiological factors, pathological factors, etc.).

Software application 470 may group the analyte profiles (e.g., daily glucose profiles) into two or more patterns based on the analysis performed by a model, for example, a machine learning model. The model is configured to identify analyte profiles (e.g., daily glucose profiles) having similar patterns and group them together. The model may identify each analyte profile (e.g., daily glucose profile) as corresponding to one of two or more patterns. The number of patterns may be small to facilitate analysis by the user and HCP. A large number of patterns, e.g., 5 or more, may be difficult for the user and HCP to interpret. The number of patterns may be based in part on the analysis period. The accuracy of the pattern identification is improved with more analyte profiles (e.g., daily glucose profiles) and provides more relevant data to the user (e.g., more statistically significant data clustering). For example, if the analysis period is three days and three patterns are assessed, with one pattern for each day, the analysis may provide relatively limited guidance to the user. Longer analysis periods, for example, of 28 or more days may include more than two patterns due to the larger amount of data. In some aspects, software application 470 (e.g., via the machine learning model) may construct a point set from the distance matrix. For example, as shown in FIGS. 6B and 6C, software application 470 may construct point set 630 from the generated distance matrix 620. In some aspects, each point of the point set may represent an analyte profile (e.g., a daily glucose profile). In some aspects, software application 470 may construct the point set using multidimensional scaling of the distance matrix. For example, as shown in FIGS. 6B and 6C, the multidimensional scaling may include translating elements of distance matrix 620 to point set 630 in a plane such that such that a distance between two points of point set 630 (e.g., point I 636 and point J 638) is the distance between a corresponding pair of the daily glucose profiles (e.g., glucose segment I 616 and glucose segment J 618).

In some aspects, software application 470 (e.g., via the machine learning model) may perform cluster analysis on the point set. For example, as shown in FIGS. 6C and 6D, software application 470 may perform cluster analysis 640 on point set 630 to identify two or more patterns (e.g., first and second patterns 646, 648). In some aspects, software application 470 may perform cluster analysis on the point set using k-means clustering. For example, the k-means clustering may utilize a centroid model that may provide efficient computation for real-time analysis, scalability for small or large datasets, and flexibility for utilizing different distance metrics. In some aspects, software application 470 may perform cluster analysis on the point set using a Gaussian mixture model. For example, the Gaussian mixture model may provide speed for fitting datasets quickly, robustness to accommodate outliers, a probabilistic approach for soft clustering and assigning different probabilities to different clusters, and flexibility to handle a wide range of complex analyte profile distributions.

In some aspects, software application 470 (e.g., via the machine learning model) may perform cluster analysis utilizing a metric to identify the two or more patterns from each other. For example, the metric may include a distance reduction ratio metric based on a separation between each pattern. In some aspects, the distance reduction ratio metric may be defined as a ratio between an average inter-pattern distance to an average intra-pattern distance, as represented by Equation (4) above. In some aspects, the distance reduction ratio metric may include a threshold above which the two or more patterns are identified. In some aspects, the threshold is at least 20%. In some aspects, the threshold is at least 25%. In some aspects, the threshold is at least 30%. In some aspects, the threshold is in a range from about 20% to about 30%. In some aspects, the threshold is in a range from about 15% to about 40%.

In some aspects, the metric may include a meal metric based on a separation between each pattern in relation to meal data of the user. In some aspects, the metric may include a medication dosing metric based on a separation between each pattern in relation to medication dosing data of the user. In some aspects, software application 470 may consider one or more metrics (e.g., distance reduction ratio metric, meal metric, and/or medication dosing metric) to identify the two or more patterns from each other during cluster analysis.

Software application 470 may utilize a machine learning model to analyze analyte profiles of analyte data of a user over an analysis period (e.g., daily glucose profiles of glucose data), and group the analyte profiles into two or more patterns based on the analysis. In some aspects, the machine learning model may be configured to streamline pattern analysis and visual representation of identified patterns. For example, as shown in FIGS. 6A-6D, machine learning model 600 may analyze analyte profiles (e.g., analyte pattern analysis 610 and distance matrix 620), and group the analyte profiles into two or more patterns (e.g., point set 630 and cluster analysis 640). In some aspects, the machine learning model may compare the analyte profiles (e.g., daily glucose profiles) and identify the two or more patterns. In some aspects, the machine learning model may include unsupervised machine learning (e.g., no training). For example, for unsupervised machine learning, software application 470 would be given the therapy data (e.g., daily glucose profiles) without any training datasets or human supervision, and allowed to identify patterns and insights based on the model without any explicit guidance or instruction.

In some aspects, the machine learning model may include supervised machine learning (e.g., with training). For example, for supervised machine learning, software application 470 would be trained with training datasets (e.g., daily glucose profiles of a specific user or population) that provide desired output values and context (e.g., bias), and then measures its accuracy using a loss function and adjusts itself iteratively until optimized. In some aspects, the supervised machine learning may include a training phase, for example, the model is given labeled data sets (e.g., labeled analyte profiles) that instruct it on which output variable is related to each input value. In some aspects, the supervised machine learning may include a testing phase, for example, the trained model is given test data (e.g., analyte profiles) that has been labeled but not revealed to the algorithm to measure how accurately the algorithm performs on unlabeled data. In some aspects, the supervised machine learning may include a classification phase, for example, the algorithm assigns test data into specific categories (e.g., analyte profiles are categorized into two or more patterns).

In some aspects, software application 470 may provide one or more recommendations to the user or a health care professional (HCP) based at least in part on two more patterns identified by the machine learning model. For example, the one or more recommendations may be presented in a report (e.g., report 472 and/or report 492) and/or in a message to the user or HCP (e.g., on receiver device 450 and/or remote device 490). In some aspects, the one or more recommendations may be generated using generative artificial intelligence (AI).

In some aspects, software application 470 may determine one or more insights of the user based on report 472. In some aspects, the one or more insights may include one or more recurring daily glucose patterns. In some aspects, the one or more insights may include a time-of-day glucose variation, a day-to-day glucose variation, elevated glucose times, elevated glucose days, a weekday variation, a weekday-to-weekend variation, a medication dosing variation, a mealtime variation, an activity variation, or a combination thereof. For example, the insight may be a pattern of high glucose levels after dinner time. In another example, the insight may be a pattern of overnight low glucose levels. In another example, the insight may be high intra-day glucose variability on weekends. In some aspects, software application 470 may provide the one or more insights in a message (e.g., message 466) and/or a report (e.g., report 472) to the user, a HCP, or both. For example, software application 470 may provide the one or more insights in message 466 and/or report 472 on receiver device 450.

In some aspects, software application 470 may analyze daily glucose profiles of glucose data of a user over an analysis period and identify correlations between glucose patterns and therapy data of the user (e.g., medication dosing data, meal data, activity data, exercise data, sleep data, stress data, etc.) by a machine learning model. In some aspects, software application 470 may provide one or more recommendations for user intervention or user action based on identified correlations between glucose patterns and therapy data. For example, the one or more recommendations may include avoiding certain meals (e.g., with high glycemic response), adjusting medication dosing for certain times of day or certain days, adjusting medication dosing for certain activities (e.g., extended exercise), getting more sleep, avoiding stressful activities, etc.

In some aspects, therapy data may include medication dosing data of the user. In some aspects, medication dosing data may include a dosing type (e.g., insulin, GLP-1, etc.), a dosing time stamp (e.g., date and time), a dosing time range (e.g., time since last dose), and/or a dose amount (e.g., 5 U) for each dosing time stamp. For example, software application 470 may consider daily medication dosing events over the analysis period, including a type of medication, dosing times and dates, dosing amounts, adherence to dosing regimen, etc., and correlate this medication dosing data with glucose patterns to identify multi-day recurring patterns associated with medication dosing data. In some aspects, software application 470 may receive medication dosing data from medication delivery device 420 (e.g., via pen cap 422) and/or user medication dosing logs.

In some aspects, therapy data may include meal data of the user. In some aspects, meal data may include a meal type, a meal description, a meal classification, a meal size, a meal time, a meal date, a meal time stamp, a meal frequency, a meal glycemic response, or other similar meal relevant information. For example, software application 470 may consider daily meal events over the analysis period, including a type of meal, meal times and dates, meal amounts, skipped meals, additional meals, etc., and correlate this meal data with glucose patterns to identify multi-day recurring patterns associated with meal data. In some aspects, software application 470 may receive meal data from one or more smart devices (e.g., first smart device 430 and/or second smart device 440) and/or user meal logs (e.g., meal logs 474).

In some aspects, therapy data may include activity data of the user. In some aspects, activity data may include exercise data, stress data, sleep data, location data, travel data, calendar data, or other similar activity relevant information. For example, software application 470 may consider daily activity events over the analysis period, including exercise events, stress events, sleep events, location events, travel events, etc., and correlate this activity data with glucose patterns to identify multi-day recurring patterns associated with activity data. In some aspects, software application 470 may receive activity data from one or more smart devices (e.g., first smart device 430 and/or second smart device 440) and/or user activity logs (e.g., activity logs 476).

In some aspects, the machine learning model of software application 470 may consider multiple therapy data (e.g., medication dosing data, meal data, and/or activity data) and correlate this multiple therapy data with glucose patterns to identify multi-day recurring patterns associated with the multiple therapy data. For example, software application 470 may quantify (e.g., rank) identified correlations with certain therapy data (e.g., meal data correlation stronger than medication dosing data, etc.), and develop personalized and optimized treatment for the user (e.g., customized recommendations for user action or adjustments to therapy).

In some aspects, the machine learning model of software application 470 may correlate the two or more patterns to one or more user parameters based on one or more metrics. In some aspects, the one or more user parameters may include medication dosing data statistics, meal data statistics, activity data statistics, exercise data statistics, stress data statistics, sleep data statistics, location data statistics, travel data statistics, calendar data statistics, daily routine statistics, or a combination thereof. In some aspects, the one or more metrics may include a statistical metric, a distance reduction ratio metric, a meal metric, a medication dosing metric, or a combination thereof.

In some aspects, software application 470 may provide one or more recommendations to the user or a HCP based at least in part on one or more correlations of the two more patterns to the one or more user parameters (e.g., confirmed by the one or more metrics) identified by the machine learning model. For example, the one or more recommendations may be presented in a report (e.g., report 472 and/or report 492) and/or in a message to the user or HCP (e.g., on receiver device 450 and/or remote device 490).

Report 472 may be configured to visually distinguish two or more patterns associated with each daily glucose profile. Report 472 may be further configured to provide one or more recommendations for user intervention or user action based on identified patterns. Report 472 may be further configured to identify correlations between glucose patterns and external events (e.g., medication dosing, meals, exercise, sleep, stress, etc.). Report 472 may be further configured to be presented on one or more devices (e.g., receiver device 450, remote server 480, remote device 490).

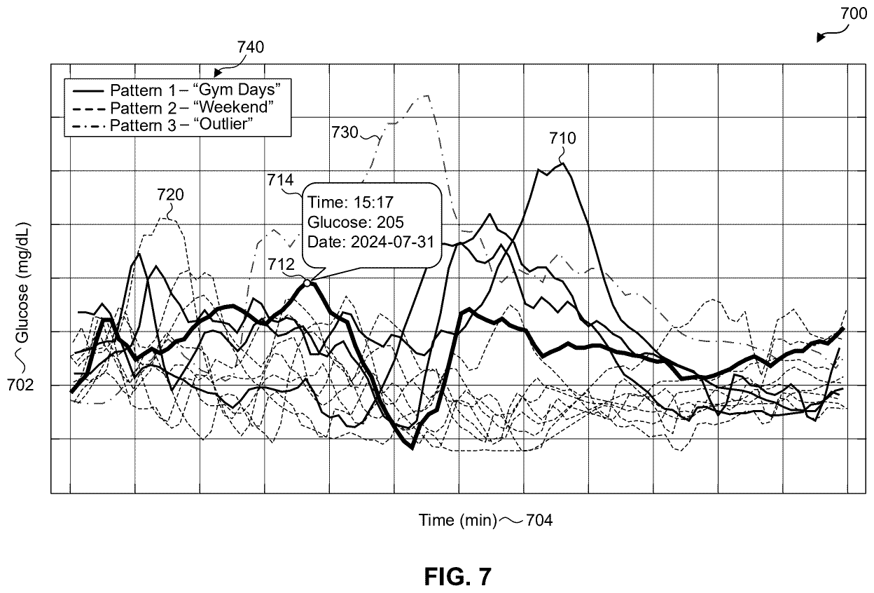

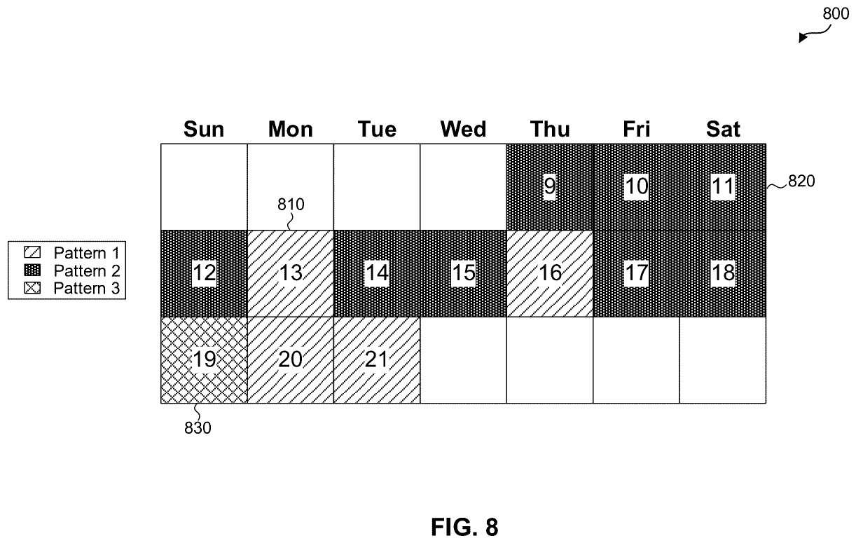

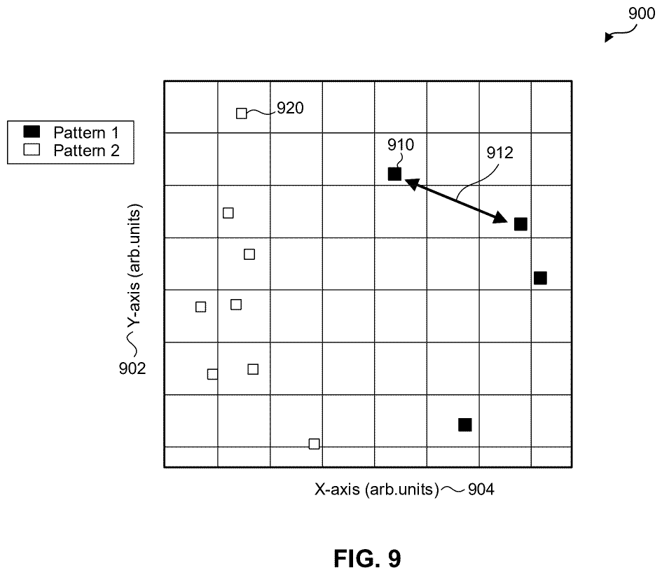

In some aspects, report 472 may include a display of each daily glucose profile. For example, as shown in FIG. 4, report 472 may include a plot 700 showing daily glucose profiles from the analysis period as a function of time. The daily glucose profiles may be displayed simultaneously, in an overlapping manner. The plot may include the identified and grouped (e.g., classified) daily glucose profiles of the user over the analysis period based on analysis performed by machine learning model. In some aspects, report 472 may include a calendar with an indication of the two or more patterns for each day on the calendar. For example, as shown in FIG. 8, report 472 may include calendar plot 800 showing daily glucose profiles as a function of calendar day, corresponding to identified and grouped (e.g., classified) daily glucose profiles of the user over the analysis period based on analysis performed by machine learning model. In some aspects, report 472 may include a graph of the two or more patterns identified. For example, as shown in FIG. 9, report 472 may include distance plot 900 showing daily glucose profiles as a function of day-to-day glucose variation on a scatter plot, corresponding to identified and grouped (e.g., classified) daily glucose profiles of the user over the analysis period based on analysis performed by machine learning model. In some aspects, report 472 may include a cluster plot 700 (FIG. 7), a calendar plot 800 (FIG. 8), a distance plot 900 (FIG. 9), or a combination thereof.

As shown in FIG. 4, analyte pattern analysis system 400 may include remote server 480. Remote server 480 may be configured to provide data management, data analysis, and/or data communication with one or more components of analyte pattern analysis system 400 (e.g., OBU 410, receiver device 450, software application 470, remote device 490, etc.). Remote server 480 may be configured to support receiver device 450, software application 470, and/or remote device 490. As shown in FIG. 4, remote server 480 may be operatively (e.g., wirelessly) coupled to receiver device 450, software application 470, and remote device 490. In some aspects, remote server 480 may include a personal computer (e.g., smartphone), a laptop computer, an external server, a server terminal, a cloud server, a web server, or other suitable server that provides functionality for other programs and/or devices.

In some aspects, remote server 480 may be connected to a wireless network (e.g., Internet), a local area network (LAN), a wide area network (WAN), or any other data network for unidirectional or bidirectional data communication between one or more components of analyte pattern analysis system 400 (e.g., OBU 410, receiver device 450, software application 470, remote device 490, etc.). In some aspects, all of software application 470 may be contained in remote server 480. In some aspects, some or part of software application 470 may be contained in remote server 480, for example, supporting processing, communication, and/or reporting functionalities of software application 470.

As shown in FIG. 4, analyte pattern analysis system 400 may include remote device 490. Remote device 490 may be configured to receive or retrieve one or more reports (e.g., report 492) from software application 470. Remote device 490 may be further configured to communicate therapy adjustments and/or recommendations to one or more components of analyte pattern analysis system 400 (e.g., receiver device 450, software application 470, remote server 480, etc.). In some aspects, remote device 490 may include a handheld computer (e.g., smartphone, cell phone, mobile phone, PDA, smart watch, etc.), personal computer, laptop computer, or any other portable communication device. As shown in FIG. 4, remote device 490 may be operatively (e.g., wirelessly, NFC, BLE, etc.) coupled to software application 470 and/or remote server 480. In some aspects, remote device 490 may receive one or more reports (e.g., report 492) from software application 470. As shown in FIG. 4, remote device 490 may include report 492, for example, for a HCP.

In some aspects, report 492 may be similar to report 472. In some aspects, report 492 may include additional information than report 472, for example, correlations between glucose patterns and external events (e.g., medication dosing, meals, exercise, sleep, stress, etc.), one or more recommendations for user intervention or user action based on identified patterns in report 492, personalized and optimized treatment recommendations for the user, or a combination thereof.

### Example Flow Diagram

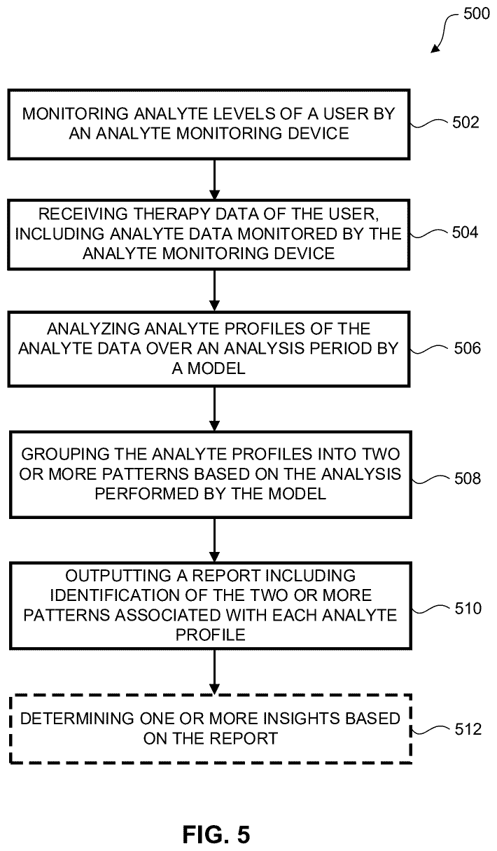

FIG. 5 illustrates flow diagram 500, according to an example aspect. For example, flow diagram 500 may be for analyte pattern analysis system 400 shown in FIG. 4. For example, flow diagram 500 may be for software application 470 shown in FIG. 4. Flow diagram 500 may be configured to identify and visualize analyte patterns (e.g., glucose patterns). Flow diagram 500 may be further configured to analyze daily glucose profiles of glucose data of a user and group the daily glucose patterns into two or more patterns. Flow diagram 500 may be further configured to output a report visually distinguishing the two or more patterns from another. Flow diagram 500 may be further configured to streamline analyte pattern analysis (e.g., glucose pattern analysis) using a machine learning model (e.g., machine learning model 600).

It is to be appreciated that not all operations in FIG. 5 are needed to perform the disclosure provided herein. Further, some of the operations may be performed simultaneously, sequentially, and/or in a different order than shown in FIG. 5. Flow diagram 500 shall be described with reference to FIGS. 4 and 6-9. However, flow diagram 500 is not limited to those example aspects. Although flow diagram 500 is shown in FIG. 5 as a stand-alone method, the aspects of this disclosure may be used with other apparatuses, systems, and/or methods, such as, but not limited to, elements in FIGS. 1-4 and 6-9, e.g., analyte monitoring system 100, sensor control device 102, receiver device 200, analyte pattern analysis system 400, machine learning model 600, cluster plot 700, calendar plot 800, and/or distance plot 900. In some aspects, flow diagram 500 may be implemented by analyte pattern analysis system 400 shown in FIG. 4 (e.g., via a processor or software application 470). In some aspects, flow diagram 500 may be implemented by software application 470 shown in FIG. 4.

In operation 502, as shown in the example of FIGS. 4 and 6-9, analyte levels of a user may be monitored by an analyte monitoring device (e.g., OBU 410 shown in FIG. 4). For example, daily glucose profiles of the user may be monitored.

In operation 504, as shown in the example of FIGS. 4 and 6-9, therapy data of the user may be received by at least one processor (e.g., receiver device 450 running software application 470 shown in FIG. 4), including analyte data monitored by the analyte monitoring device. For example, the therapy data may include analyte data of the user, for example, glucose data (e.g., daily glucose profiles).

In some aspects, the therapy data may include medication dosing data (e.g., via pen cap 422 shown in FIG. 4). In some aspects, for example, pen cap 422 may detect a dosing event of injection pen 426 (e.g., insulin, GLP-1, etc.), for example, the dosing event may be inferred from a decapping event of pen cap 422 from injection pen 426 and a capping event of pen cap 422 to injection pen 426. In some aspects, the therapy data may include meal data (e.g., via meal logs 474 shown in FIG. 4). In some aspects, for example, meal logs 474 and/or one or more smart devices (e.g., first smart device 430 and/or second smart device 440 shown in FIG. 4) may detect a meal event of the user (e.g., breakfast, lunch, dinner, snack). In some aspects, the therapy data may include activity data, exercise data, stress data, sleep data, location data, travel data, calendar data, or a combination thereof (e.g., via activity logs 476 shown in FIG. 4). In some aspects, for example, activity logs 476 and/or one or more smart devices (e.g., first smart device 430, second smart device 440, and/or receiver device 450 shown in FIG. 4) may detect an activity event of the user, an exercise event of the user, a stress event of the user, a sleep event of the user, a location event of the user, a travel event of the user, or a combination thereof.

In operation 506, as shown in the example of FIGS. 4 and 6-9, analyte profiles (e.g., glucose segment I 616 and glucose segment J 618 shown in FIG. 6A) of the analyte data over an analysis period (e.g., 14 days) may be analyzed by a machine learning model (e.g., machine learning model 600 shown in FIGS. 6A-6D). For example, the analyte profiles may include daily glucose profiles.

In some aspects, each of the analyte profiles may include analyte data of the user over a 24-hour time window (e.g., daily analyte profiles). In some aspects, each of the analyte profiles may include analyte data of the user over less than a 24-hour time window. In some aspects, each of the analyte profiles may include analyte data segments, for example, a breakfast time window, a lunch time window, a dinner time window, a snack time window, or a combination thereof. In some aspects, the analysis period may be in a range from 3 days to 14 days. In some aspects, the analysis period may be in a range from 3 days to 30 days. In some aspects, the analysis period may be at least 7 days.

In some aspects, the machine learning model may receive the analyte profiles (e.g., daily glucose profiles) and group them into patterns. The grouping may be performed based on one or more metrics for each analyte profile (e.g., daily glucose profile). In some aspects, the one or more metrics may include a similar mean glucose. In some aspects, the one or more metrics may include a similar glucose variability. In some aspects, the one or more metrics may include a minimum peak glucose level. In some aspects, the machine learning model may include assessing a distance between the daily glucose profiles. For example, the distance may be based on a MARD (e.g., Equation 3 above). In some aspects, first and second daily glucose profiles (e.g., glucose segment J 618 shown in FIG. 6A) may be grouped into a first pattern type (e.g., first pattern 646) when the distance is at or below a threshold. For example, the threshold may be based on a predetermined analyte value (e.g., glucose level of 30 mg/dL, glucose difference of 30 mg/dL). In some aspects, the machine learning model may include unsupervised machine learning (e.g., no training). In some aspects, the machine learning model may include supervised machine learning (e.g., with training).

In operation 508, as shown in the example of FIGS. 4 and 6-7, the daily analyte profiles may be grouped into two or more patterns (e.g., first and second patterns 646, 648 shown in FIG. 6D) based on the analysis performed by the machine learning model.

In some aspects, the grouping may be limited to no greater than three patterns. For example, when the analysis period is in a range from 3 days to 14 days, the grouping is limited to no more than three patterns (e.g., minimum data points for cluster analysis).

In operation 510, as shown in the example of FIGS. 4 and 6-7, a report (e.g., report 472 shown in FIG. 4) may be outputted to a display device (e.g., receiver device 450 and/or remote device 490 shown in FIG. 4) in communication with the at least one processor, the report including identification of the two or more patterns associated with each daily analyte profile (e.g., cluster plot 700 shown in FIG. 7, calendar plot 800 shown in FIG. 8, and/or distance plot 900 shown in FIG. 9).

In some aspects, the report may include a display of each daily analyte profile. For example, as shown in FIG. 7, the report may include cluster plot 700 that overlays first, second, and third patterns 710, 720, 730 over the analysis period and visually distinguishes each pattern from another (e.g., by color, by size, by shape, etc.). In some aspects, the report may include a calendar with an indication of the two or more patterns for each day on the calendar. For example, as shown in FIG. 8, the report may include calendar plot 800 that separates first, second, and third patterns 810, 820, 830 into corresponding calendar days and visually distinguishes each pattern from another (e.g., by color, by size, by shape, etc.). In some aspects, the report may include a graph of the two or more patterns. For example, as shown in FIG. 9, the report may include distance plot 900 that maps the two or more patterns onto a plane (e.g., scatter plot) and visually distinguishes each pattern from another (e.g., by color, by size, by shape, etc.). In some aspects, distance plot 900 may represent a day-to-day analyte variation between each of the daily analyte profiles. In some aspects, the report may include cluster plot 700 (FIG. 7), calendar plot 800 (FIG. 8), distance plot 900 (FIG. 9), or a combination thereof.

In operation 512, optionally, as shown in the example of FIGS. 4 and 6-7, one or more insights of the user may be determined based on the report.

In some aspects, the one or more insights may include one or more recurring analyte patterns (e.g., first pattern 646 shown in FIG. 6D). In some aspects, the one or more insights may include one or more of a time-of-day analyte variation, a day-to-day analyte variation, elevated analyte times, elevated analyte days, a weekday variation, a weekday-to-weekend variation, a medication dosing variation, a mealtime variation, or an activity variation. In some aspects, flow diagram 500 may include providing the one or more insights in a message to the user (e.g., via message 466 and/or report 472 on receiver device 450 shown in FIG. 4), a HCP (e.g., via remote device 490 shown in FIG. 4), or both. In some aspects, flow diagram 500 may further include providing a recommendation to the user (e.g., via message 466 and/or report 472 on receiver device 450 shown in FIG. 4) or a HCP (e.g., via a message and/or report 492 on remote device 490 shown in FIG. 4) based at least in part on the two or more patterns.

In some aspects, flow diagram 500 may include correlating the two or more patterns to one or more user parameters based on one or more metrics (e.g., via the machine learning model). In some aspects, for example, the one or more user parameters may include one or more of medication dosing data statistics (e.g., via pen cap 422 shown in FIG. 4), meal data statistics (e.g., via meal logs 474 shown in FIG. 4), activity data statistics (e.g., via activity logs 476 shown in FIG. 4), exercise data statistics, stress data statistics, sleep data statistics, location data statistics, travel data statistics, calendar data statistics, or daily routine statistics. In some aspects, for example, the one or more metrics may include one or more of a statistical metric, a distance reduction ratio metric, a meal metric, or a medication dosing metric. In some aspects, flow diagram 500 may further include providing a recommendation to the user (e.g., via message 466 and/or report 472 on receiver device 450 shown in FIG. 4) or a HCP (e.g., via a message and/or report 492 on remote device 490 shown in FIG. 4) based on a correlation of the two or more patterns to the one or more user parameters.

### Example Machine Learning Model

FIGS. 6A-6D illustrate machine learning model 600, according to an example aspect. For example, machine learning model 600 may be for software application 470 of analyte pattern analysis system 400 shown in FIG. 4. For example, machine learning model 600 may be for flow diagram 500 shown in FIG. 5. Machine learning model 600 may be configured to identify and visualize analyte patterns. Machine learning model 600 may be further configured to analyze analyte profiles of analyte data of a user and group the analyte patterns into two or more patterns. Machine learning model 600 may be further configured to streamline analyte pattern analysis using one or more algorithms (e.g., unsupervised machine learning).

It is to be appreciated that not all operations in FIGS. 6A-6D are needed to perform the disclosure provided herein. Further, some of the operations may be performed simultaneously, sequentially, and/or in a different order than shown in FIGS. 6A-6D. Machine learning model 600 shall be described with reference to FIGS. 4, 5, and 7-9. However, machine learning model 600 is not limited to those example aspects. Although machine learning model 600 is shown in FIGS. 6A-6D as a stand-alone model and/or process, the aspects of this disclosure may be used with other apparatuses, systems, and/or methods, such as, but not limited to, elements in FIGS. 1-5 and 7-9, e.g., analyte monitoring system 100, sensor control device 102, receiver device 200, analyte pattern analysis system 400, flow diagram 500, cluster plot 700, calendar plot 800, and/or distance plot 900. In some aspects, machine learning model 600 may be implemented by analyte pattern analysis system 400 shown in FIG. 4 (e.g., via a processor or software application 470). In some aspects, machine learning model 600 may be implemented in flow diagram 500 shown in FIG. 5 (e.g., via a processor or app).

As shown in FIGS. 6A-6D, machine learning model 600 may include analyte pattern analysis 610, distance matrix 620, point set 630, and cluster analysis 640. In some aspects, the various components of machine learning model 600, and the one or more software modules that make them up (e.g., receiver device 450, software application 470, remote server 480, etc.), may be implemented on a single processor, multiple processors, a single server, multiple web-servers, and/or intranet servers.

Analyte pattern analysis 610 may be configured to analyze (e.g., compare) pairs of analyte profiles. For example, analyte pattern analysis 610 may analyze pairs of daily glucose profiles. Analyte pattern analysis 610 may be further configured to calculate a distance (e.g., MARD) between each pair of analyte profiles (e.g., each pair of daily glucose profiles). Analyte pattern analysis 610 may be further configured to generate a distance matrix 620 based on a distance between each pair of analyte profiles. As shown in FIG. 6A, analyte pattern analysis 610 may include glucose level (mg/dL) 612 as a function of time (min) 614 for pairs of analyte profiles of analyte data (e.g., daily glucose profiles of glucose data) of a user over an analysis period (e.g., monitored by OBU 410), for example, glucose segment I 616 (e.g., indicated by a solid line) and glucose segment J 618 (e.g., indicated by a dashed line). In some aspects, glucose level (mg/dL) 612 may include a glucose difference between pairs of analyte profiles of analyte data (e.g., daily glucose profiles of glucose data).

As shown in FIGS. 6A and 6B, analyte pattern analysis 610 may perform a distance calculation 619 for each pair of analyte profiles (e.g., between glucose segment I 616 and glucose segment J 618) to generate a corresponding distance element (e.g., distance Dᵢⱼ 626) in distance matrix 620. In some aspects, the distance calculation 619 may be defined by a metric, for example, a MAD, a median absolute difference, a WMAD, a weighted median absolute difference, a MARD, a median absolute relative difference, a PARD, an absolute difference, an absolute deviation, a standard deviation, or any other suitable difference metric.

In some aspects, as shown in FIG. 6A, analyte pattern analysis 610 may include a glucose threshold Δg 613, which represents an allowable spread or dispersion in glucose level from the daily glucose profiles (e.g., glucose segment I 616). In some aspects, for example, glucose threshold Δg 613 may be adjusted to a specified value (e.g., Δg=0%, ±1%, ±2%, ±5%, ±10%, etc. from the baseline glucose value of the corresponding daily glucose profile). In some aspects, as shown in FIG. 6A, analyte pattern analysis 610 may include a time threshold Δt 615, which represents an allowable spread or dispersion in time from the daily glucose profiles (e.g., glucose segment I 616). In some aspects, for example, time threshold Δt 615 may be adjusted to a specified value (e.g., Δt=0%, ±1%, ±2%, ±5%, ±10%, etc. from the baseline time value of the corresponding daily glucose profile).

Distance matrix 620 may be configured to represent the distances, taken pairwise, between analyte profiles (e.g., daily glucose profiles) over an analysis period. Distance matrix 620 may be further configured to represent the distance between each pair of N analyte profiles for an N×N symmetric matrix (2D array). As shown in FIG. 6B, distance matrix 620 may include row I 622, column J 624, and distance Dᵢⱼ 626 corresponding to a distance (e.g., distance calculation 619) between glucose segment I 616 and glucose segment J 618. Distance matrix 620 may include a corresponding distance Dᵢⱼ 626 (e.g., specific row and column element) calculated between each pair of analyte profiles (e.g., each pair of daily glucose profiles).

Point set 630 may be configured to represent (e.g., a lower-dimensional representation) elements of the distance matrix 620 (e.g., distance Dᵢⱼ 626) as a set of points in a plane (e.g., a scatter plot). Point set 630 may be further configured to translate elements of the distance matrix 620 (e.g., distance Dᵢⱼ 626) into a plane such that a distance between two points of point set 630 (e.g., point I 636 and point J 638) is the calculated distance between a corresponding pair of analyte profiles (e.g., glucose segment I 616 and glucose segment J 618). As shown in FIG. 6C, point set 630 may include Y-axis (arb. units) 632, X-axis (arb. units) 634, point 1636 (e.g., representing glucose segment I 616), and point J 638 (e.g., representing glucose segment J 618). In some aspects, as shown in FIG. 6C, point set 630 may be represented as a scatter plot using Cartesian coordinates (Y-axis 632, X-axis 634) to display the collection of points (e.g., point I 636, point J 638, etc.) at corresponding horizontal (X-axis) and vertical (Y-axis) positions.

In some aspects, point set 630 may be constructed from distance matrix 620 by multidimensional scaling. For example, the multidimensional scaling (MDS) may include an algorithm (e.g., classical MDS, mMDS, NMDS, GMDS) to place each analyte profile (e.g., glucose segment I 616 and glucose segment J 618) into an N-dimensional space (e.g., N=2 for a 2D scatter plot) such that the distances (e.g., distance Dᵢⱼ 626) between the analyte profiles is preserved in the distances between points of point set 630 (e.g., point I 636 and point J 638).

Cluster analysis 640 may be configured to group points of point set 630 into two or more patterns such that points (e.g., daily glucose profiles) of each pattern are more similar to each other than to those in other patterns. Cluster analysis 640 may be further configured to utilize a metric to identify (e.g., classify) two or more patterns of point set 630 from each other. As shown in FIG. 6D, cluster analysis 640 may include Y-axis (arb. units) 642, X-axis (arb. units) 644, first pattern 646 (e.g., including point J 638), and second pattern 648 (e.g., including point 1636). Cluster analysis 640 may group points of point set 630 (e.g., point I 636 and point J 638), representing each of the daily analyte profiles (e.g., glucose segment I 616 and glucose segment J 618), into first and second patterns 646, 648.

In some aspects, cluster analysis 640 may include one or more algorithms to separate point set 630 into two or more patterns (e.g., first and second patterns 646, 648). For example, the one or more algorithms may include a centroid model (e.g., k-means clustering), a model-based clustering (e.g., a Gaussian mixture model), a connectivity model (e.g., hierarchical clustering), self-organizing mapping (e.g., unsupervised neural network), or any other suitable clustering algorithm. In some aspects, cluster analysis 640 may utilize k-means clustering. In some aspects, cluster analysis 640 may utilize a Gaussian mixture model.

In some aspects, cluster analysis 640 may utilize a metric to identify (e.g., classify) the two or more patterns from each other (e.g., first and second patterns 646, 648). For example, the metric may include a distance reduction ratio, a Dunn index, a Davies-Bouldin index, a silhouette coefficient, a purity, a Rand index, an F-measure, a Jaccard index, a Dice index, a Fowlkes-Mallows index, a Chi index, a confusion matrix, a Hopkins statistic, or any other suitable metric. In some aspects, cluster analysis 640 may utilize a distance reduction ratio. In some aspects, the metric may include a threshold above which the two or more patterns are identified (e.g., classified). For example, for the distance reduction ratio metric, the threshold may be at least 20%, at least 25%, or at least 30%. In some aspects, the threshold is in a range from about 20% to about 30%. In some aspects, the threshold is in a range from about 15% to about 40%.

In some aspects, cluster analysis 640 may utilize one or more metrics to identify (e.g., classify) the two or more patterns from each other (e.g., first and second patterns 646, 648) based on therapy data of the user. For example, cluster analysis 640 may utilize a meal metric based on a separation between each pattern in relation to meal data of the user. For example, cluster analysis 640 may utilize a medication dosing metric based on a separation between each pattern in relation to medication dosing data of the user. For example, cluster analysis 640 may utilize an activity metric based on a separation between each pattern in relation to activity data of the user. In some aspects, cluster analysis 640 may utilize the one or more metrics (e.g., meal metric, medication dosing metric, activity metric, etc.) separately or in combination. In some aspects, cluster analysis 640 may utilize the one or more metrics (e.g., meal metric, medication dosing metric, activity metric, etc.) separately or in combination with the above referenced metrics (e.g., a distance reduction ratio).

### Example Report

FIGS. 7-9 illustrate example plots 700, 800, 900 of report 472 from software application 470, according to various example aspects. FIG. 7 shows a plot 700 of daily glucose profiles as a function of time visually indicating first, second, and third patterns 710, 720, 730, according to an example aspect. However, in other aspects additional or fewer patterns may be identified, e.g., 2 patterns, 4 patterns, etc. FIG. 8 shows calendar plot 800 of daily glucose profiles as a function of calendar day visually indicating first, second, and third patterns 810, 820, 830, according to an example aspect. FIG. 9 shows distance plot 900 of daily glucose profiles as a function of day-to-day glucose variation visually indicating first and second patterns 910, 920, according to an example aspect.

Although plots 700, 800, 900 are shown in FIGS. 7-9 as a stand-alone output, apparatus and/or system, the aspects of this disclosure may be used with other apparatuses, systems, and/or methods, such as, but not limited to, elements in FIGS. 1-6D, e.g., analyte monitoring system 100, sensor control device 102, receiver device 200, analyte pattern analysis system 400, flow diagram 500, and/or machine learning model 600.

As shown in FIG. 7, cluster plot 700 shows glucose levels (mg/dL) 702 as a function of time (min) 704 for first, second, and third patterns 710, 720, 730, corresponding to identified and grouped (e.g., classified) daily glucose profiles of a user over an analysis period based on analysis performed by machine learning model 600. Cluster plot 700 shows first pattern 710 (e.g., indicated by a solid line) that includes four grouped daily glucose profiles, second pattern 720 (e.g., indicated by a dashed line) that includes eight grouped daily glucose profiles, and third pattern 730 (e.g., indicated by a dashed-dot line) that includes one grouped daily glucose profile (e.g., an outlier).

In some aspects, as shown in FIG. 7, a daily glucose profile may be selected in cluster plot 700 of report 472, shown by indicator 712, to provide further information about the selected daily glucose profile. For example, as shown in FIG. 7, indicator 712 may be adjusted (e.g., slide) along the selected daily glucose profile and provide information display 714, for example, providing the time of day, the glucose value, and date of the selected daily glucose profile. In some aspects, as shown in FIG. 7, cluster plot 700 may overlay first, second, and third patterns 710, 720, 730 over the analysis period and visually distinguish each pattern from another. For example, cluster plot 700 may visually distinguish each pattern from another by color (e.g., first pattern 710 in red, second pattern 720 in blue, third pattern 730 in grey). In some aspects, cluster plot 700 may include a legend to provide one or more insights about the identified patterns. For example, as shown in FIG. 7, cluster plot 700 may include legend 740 with insights for each pattern, for example, legend 740 may include Pattern 1 (e.g., first pattern 710) with “Gym Days,” Pattern 2 (e.g., second pattern 720) with “Weekend,” and Pattern 3 (e.g., third pattern 730) with “Outlier.” Patterns may be distinguished by other means, such as using dotted lines, lines of different thickness, or the glucose profiles may be displayed by a series of data points with each pattern having data points represented by a different shape or icon (e.g., circle, triangle, square). In some aspects, the user may be able to select a pattern and plot 700 displays the glucose profiles identified as having that pattern, and the other patterns may be hidden or otherwise deemphasized.

The plot 700 allows the user to see which days correspond to a particular pattern. For example, pattern 1 may correspond to weekends, whereas pattern 2 may correspond to weekdays. This may allow the user to more easily determine how his or her behaviors on certain days impact glucose levels. This may also guide the HCP to ask about the user's behaviors on the days having a pattern indicative of glucose dysregulation and provide recommendation as to behavioral modifications or medication adjustments.

In some aspects, the report may include insights and recommendations based on one or more of the patterns. For example, the system may output an insight that glucose levels are high at dinner time in pattern 1. In some aspects, the insights and recommendations may be generated based on generative AI. For example, the generative AI may generate insights or recommendations on how to improve outcomes on certain days of a particular pattern based on inputs (e.g., prompts) that include collected data (e.g., CGM data, activity data, meal data, etc.) for that particular pattern. The system may provide a recommendation to consider alternate meal choices at dinner time. The pattern may indicate glucose control and the insight may indicate that the user has good glucose control on certain days. This may allow the user to consider what behaviors or actions the user takes on the days assessed to have a pattern indicative of good glucose control and contrast with days having a different pattern type.

As shown in FIG. 8, calendar plot 800 shows the identified pattern for each calendar day. Calendar plot 800 shows daily glucose profiles as a function of calendar day for first, second, and third patterns 810, 820, 830, corresponding to identified and grouped (e.g., classified) daily glucose profiles of a user over an analysis period based on analysis performed by machine learning model 600. Calendar may display the days of the analysis period. In FIG. 8, the analysis period included the 9^{th} through 21^{st} of the month. Each calendar day may indicate the pattern for that day. This allows the user to easily see which days share a similar glucose pattern. For example, in FIG. 8, the user may notice that he or she has the same glucose pattern on Mondays, and the user has a different glucose pattern on Fridays and Saturdays each week. The first pattern may be, for example, a pattern of glucose control and the user may see which days have the pattern indicative of glucose control. The second pattern may indicate glucose dysregulation, and the user may easily see which days have a pattern of glucose dysregulation, such as Fridays/Saturdays, so that the user may make modifications accordingly. As discussed herein, the system may generate an insight about the pattern and/or the timing of the pattern. For example, if pattern 2 may be characterized by high glucose variability, and pattern 2 is present on each Friday/Saturday, system may output an indication of a pattern of high glucose variability on Friday/Saturday. The system may recommend monitoring food choices and/or medication dosing amounts on those days, or encouraging healthy dieting.

Calendar plot 800 shows first pattern 810 (e.g., indicated by cross horizontal line calendar days) that includes four grouped daily glucose profiles (e.g., 13^{th}, 16^{th}, 20^{th}, 21^{st}) second pattern 820 (e.g., indicated by dashed calendar days) that includes eight grouped daily glucose profiles (e.g., 9^{th}, 10^{th}, 11^{th}, 12^{th}, 14^{th}, 15^{th}, 17^{th}, 18^{th}), and third pattern 830 (e.g., indicated by cross-hatched calendar day) that includes one grouped daily glucose profile (e.g., 19^{th}). As shown in FIG. 8, the system may visually indicate that the user experienced pattern 1 on certain weekdays (e.g., Monday, Tuesday, Thursday) while the user experienced pattern 2 on the other weekdays and weekends. The system may utilize this information to provide one or more recommendations to the user on those certain calendar day to modify therapy, for example, modifying eating habits and/or activities on Mondays to reduce glucose levels of pattern 1 in the evenings and on Fridays to reduce glucose levels of pattern 2 in the morning.

In some aspects, as shown in FIG. 8, calendar plot 800 may include first, second, and third patterns 810, 820, 830 for each calendar day over the analysis period and visually distinguish each pattern from another. For example, calendar plot 800 may visually distinguish each pattern from another by color (e.g., first pattern 810 in red, second pattern 820 in blue, third pattern 830 in grey). In some aspects, first, second, and third patterns 810, 820, 830 of calendar plot 800 shown in FIG. 8 may be the same daily glucose profiles identified as first, second, and third patterns 710, 720, 730 of cluster plot 700 shown in FIG. 7. In some aspects, report 472 may include cluster plot 700 and calendar plot 800 adjacent to each other.

As shown in FIG. 9, distance plot 900 shows daily glucose profiles as a function of day-to-day glucose variation on a scatter plot (e.g., Y-axis 902, X-axis 904) for first and second patterns 910, 920, corresponding to identified and grouped (e.g., classified) daily glucose profiles of a user over an analysis period based on analysis performed by machine learning model 600. Distance plot 900 may be generated by software application 470 similar to how point set 630 shown in FIG. 6C was generated (e.g., via MDS of distance matrix 620). For example, the model may translate elements of a distance matrix (e.g., distance Dᵢⱼ 626) into a plane (e.g., scatter plot), via MDS, such that a distance 912 between two points of distance plot 900 is the calculated distance between a corresponding pair of analyte profiles (e.g., daily glucose profiles). Distance plot 900 shows first pattern 910 (e.g., indicated by solid black squares) that includes four grouped daily glucose profiles, and second pattern 920 (e.g., indicated by open black squares) that includes eight grouped daily glucose profiles. In some aspects, distance plot 900 may omit any outliers (e.g., third pattern 730 shown in FIG. 7).

In some aspects, as shown in FIG. 9, a pair of daily glucose profiles may be selected in distance plot 900 of report 472, shown by distance 912, to provide a distance between the selected pair of daily glucose profiles (e.g., mean absolute difference). In some aspects, as shown in FIG. 9, distance plot 900 may graph (e.g., map) first and second patterns 910, 920 based on a distance between each pair of daily glucose profiles and visually distinguish each pattern from another. For example, distance plot 900 may visually distinguish each pattern from another by color (e.g., first pattern 910 in red, second pattern 920 in blue). In some aspects, first and second patterns 910, 920 of distance plot 900 shown in FIG. 9 may be the same daily glucose profiles identified as first and second patterns 710, 720 of cluster plot 700 shown in FIG. 7. In some aspects, report 472 may include cluster plot 700 and distance plot 900 adjacent to each other.

In some aspects, report 472 may include cluster plot 700, calendar plot 800, distance plot 900, or a combination thereof.

### Example Model for Titration Profiles

As discussed above, a problem in the art is that PwD exhibit large glycemic variability and day-to-day glucose deviations, even with prescribed therapies (e.g., oral medication, insulin dosing regimen, GLP-1 therapy, etc.), due to glucose levels being affected by multiple factors dependent upon daily routine, profession, activities, and self-care (e.g., medication dosing, meals, exercise, stress, hormones, etc.). Also, current daily monitoring schemes (e.g., AGP) do not contemplate analyzing pattern variations from day-to-day to identify distinct user patterns over time. Further, current daily monitoring schemes do not consider different daily habits and routines of individuals to identify multiple patterns and generate tailored titration profiles based on those identified patterns (e.g., weekday vs. weekend, day shift vs. night shift, not traveling vs. traveling, etc.).

In order to address this problem, the systems and methods of the present disclosure identify two or more patterns associated with an individual's multi-day analyte data (e.g., glucose data) and generate improved titration profiles for each of the identified patterns. This provides an improvement to diabetes management technology by correlating the analyte data to a specific habit or routine of an individual and optimizing a titration profile for that specific pattern. For example, the system may identify a first analyte pattern (e.g., weekday, day shift, not traveling, etc.) distinguishable from a second analyte pattern (e.g., weekend, night shift, traveling, etc.), and then generate a corresponding first titration profile based on the first analyte pattern and a second titration profile based on the second analyte pattern.

Further, the present disclosure may utilize a generative AI model to determine whether analyte patterns are correlated to a first type of user data (e.g., weekdays, weekends, day shift, night shift, not traveling, traveling, etc.) to generate and optimize a distinct titration profile for each of the identified patterns. For example, the system may utilize a LLM to compare identified analyte patterns to one or more types of user data to determine a level of correlation, and then generate an improved titration profile based on the level of correlation for a specific identified pattern. Additionally, the present disclosure may dynamically update a titration profile in real time based on a variability of that titration profile over time, which improves diabetes management technology by continually adjusting and maintaining the titration profile within an optimal target range (e.g., glucose level of 100 mg/dL).

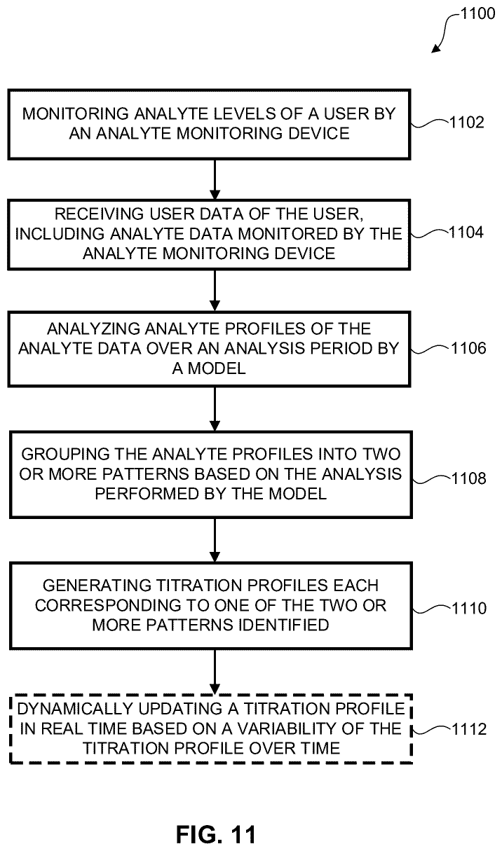

FIGS. 10A-10C illustrate model 1000, according to an example aspect. For example, model 1000 may be for software application 470 of analyte pattern analysis system 400 shown in FIG. 4. For example, model 1000 may be for flow diagram 1100 shown in FIG. 11. Model 1000 may be configured to identify analyte patterns. Model 1000 may be further configured to analyze analyte profiles of analyte data of a user and group the analyte patterns into two or more patterns. Model 1000 may be further configured to develop personalized and optimized titration profiles for adaptive dose guidance for an individual. Model 1000 may be further configured to identify one or more patterns (e.g., weekdays vs. weekends, day shift vs. night shift, not traveling vs. traveling, etc.) and generate one or more titration profiles for each of the identified patterns. Model 1000 may be further configured to utilize a generative AI model to determine whether analyte patterns are correlated to a first type of user data (e.g., weekdays, weekends, day shift, night shift, not traveling, traveling, etc.). Model 1000 may be further configured to utilize a LLM to compare identified analyte patterns to one or more types of user data to determine a level of correlation. Model 1000 may be further configured to streamline analyte pattern analysis using one or more algorithms (e.g., unsupervised machine learning, LLM).

It is to be appreciated that not all operations in FIGS. 10A-10C are needed to perform the disclosure provided herein. Further, some of the operations may be performed simultaneously, sequentially, and/or in a different order than shown in FIGS. 10A-10C. Model 1000 shall be described with reference to FIGS. 4-9 and 11. However, model 1000 is not limited to those example aspects. Although model 1000 is shown in FIGS. 10A-10C as a stand-alone model and/or process, the aspects of this disclosure may be used with other apparatuses, systems, and/or methods, such as, but not limited to, elements in FIGS. 1-9 and 11-15, e.g., analyte monitoring system 100, sensor control device 102, receiver device 200, analyte pattern analysis system 400, flow diagram 500, machine learning model 600, cluster plot 700, calendar plot 800, distance plot 900, flow diagram 1100, model 1200, flow diagram 1300, flow diagram 1400, and/or flow diagram 1500. In some aspects, model 1000 may be implemented by analyte pattern analysis system 400 shown in FIG. 4 (e.g., via a processor or software application 470). In some aspects, model 1000 may be implemented in flow diagram 1100 shown in FIG. 11 (e.g., via a processor or app).

The aspects of machine learning model 600 shown in FIGS. 6A-6D, for example, and the aspects of model 1000 shown in FIGS. 10A-10C may be similar. Similar reference numbers are used to indicate features of the aspects of machine learning model 600 shown in FIGS. 6A-6D and the similar features of the aspects of model 1000 shown in FIGS. 10A-10C. One difference between the aspects of machine learning model 600 shown in FIGS. 6A-6D and the aspects of model 1000 shown in FIGS. 10A-10C is that model 1000 generates first titration profile 1066 and second titration profile 1086 from cluster analysis 1040 of first pattern 1046 and second pattern 1048, respectively, instead of or in addition to generating a report (e.g., report 472 with cluster plot 700, calendar plot 800, and distance plot 900) from cluster analysis 640 of machine learning model 600 shown in FIGS. 6A-6D.

Model 1000 is similar to machine learning model 600 shown in FIGS. 6A-6D and similar reference numbers are used to indicate the similar features of machine learning model 600 shown in FIGS. 6A-6D and model 1000 shown in FIGS. 10A-10C. Discussion of model 1000 components, processes, properties, and/or functionality (e.g., analyte pattern analysis 1010, distance matrix 620 shown in FIG. 6B, point set 630 shown in FIG. 6C, cluster analysis 1040) is not duplicated here for brevity, but the aspects and features of each are similar to machine learning model 600 shown in FIGS. 6A-6D described above.

As shown in FIGS. 10A-10C, model 1000 may include analyte pattern analysis 1010, cluster analysis 1040, and first and second titration profiles 1066, 1086. In some aspects, the various components of model 1000, and the one or more software modules that make them up (e.g., receiver device 450, software application 470, remote server 480, etc.), may be implemented on a single processor, multiple processors, a single server, multiple web-servers, and/or intranet servers.

Analyte pattern analysis 1010 may be configured to analyze (e.g., compare) pairs of analyte profiles. For example, analyte pattern analysis 1010 may analyze pairs of daily glucose profiles. Analyte pattern analysis 1010 may be further configured to calculate a distance (e.g., MARD) between each pair of analyte profiles (e.g., each pair of daily glucose profiles). In some aspects, analyte pattern analysis 1010 may be further configured to generate a distance matrix (e.g., similar to distance matrix 620 shown in FIG. 6B) based on a distance between each pair of analyte profiles. In some aspects, analyte pattern analysis 1010 may be further configured to generate a point set (e.g., similar to point set 630 shown in FIG. 6C) constructed from the distance matrix (e.g., similar to distance matrix 620 shown in FIG. 6B) by multidimensional scaling. In some aspects, analyte pattern analysis 1010 may be further configured generate a point set (e.g., similar to point set 630 shown in FIG. 6C) to represent (e.g., a lower-dimensional representation) elements of the distance matrix (e.g., similar to distance Dᵢⱼ 626 shown in FIG. 6B) as a set of points in a plane (e.g., a scatter plot). In some aspects, analyte pattern analysis 1010 may be further configured to provide the point set (e.g., similar to point set 630 shown in FIG. 6C) to cluster analysis 1040 to group points of the point set into two or more patterns.

As shown in FIG. 10A, analyte pattern analysis 1010 may include glucose level (mg/dL) 1012 as a function of time (min) 1014 for pairs of analyte profiles of analyte data (e.g., daily glucose profiles of glucose data) of a user over an analysis period (e.g., monitored by OBU 410), for example, glucose segment I 1016 (e.g., indicated by a solid line) and glucose segment J 1018 (e.g., indicated by a dashed line). In some aspects, glucose level (mg/dL) 1012 may include a glucose difference between pairs of analyte profiles of analyte data (e.g., daily glucose profiles of glucose data).

In some aspects, as shown in FIG. 10A, analyte pattern analysis 1010 may include a glucose threshold Δg 1013, which represents an allowable spread or dispersion in glucose level from the daily glucose profiles (e.g., glucose segment I 1016). In some aspects, for example, glucose threshold Δg 1013 may be adjusted to a specified value (e.g., Δg=0%, ±1%, ±2%, ±5%, ±10%, etc. from the baseline glucose value of the corresponding daily glucose profile). In some aspects, as shown in FIG. 10A, analyte pattern analysis 1010 may include a time threshold Δt 1015, which represents an allowable spread or dispersion in time from the daily glucose profiles (e.g., glucose segment I 1016). In some aspects, for example, time threshold Δt 1015 may be adjusted to a specified value (e.g., Δt=0%, ±1%, ±2%, ±5%, ±10%, etc. from the baseline time value of the corresponding daily glucose profile).

In some aspects, as shown in FIG. 10A, model 1000 may also include user data 1020 to be used in cluster analysis 1040. In some aspects, user data 1020 may include timing data, calendar data, user schedule data, user work data, travel data, GPS location data, time zone data, or a combination thereof. In some aspects, for example, user data 1020 may include timing data or calendar data to determine whether the glucose profiles occur on the weekdays or on the weekends. In some aspects, for example, user data 1020 may include user schedule data or user work data to determine whether the glucose profiles occur during a day shift or a night shift. In some aspects, for example, user data 1020 may include user travel data, GPS location data, or time zone data to determine whether the glucose profiles occur when the user is traveling or not traveling.

In some aspects, user data 1020 may be received via a user query. In some aspects, model 1000 may include outputting a query to the user requesting user data 1020 (e.g., via receiver device 450 shown in FIG. 4). In some aspects, user data 1020 may be received from the user based on a response to the query. In some aspects, user data 1020 may be determined based on the glucose profiles. In some aspects, for examples, the glucose profiles may be timestamped such that model 1000 may determine if the glucose profiles occur on the weekdays or the weekends, during a day shift or a night shift, or when the user is traveling or not traveling. In some aspects, user data 1020 may be determined via one or more monitoring devices (e.g., first smart device 430, second smart device 440, and/or receiver device 450 shown in FIG. 4).

Cluster analysis 1040 may be configured to group points of a point set (e.g., similar to point set 630 shown in FIG. 6C) generated by analyte pattern analysis 1010 into two or more patterns, such that the points (e.g., daily glucose profiles) of each pattern are more similar to each other than to those in other patterns. Cluster analysis 1040 may be further configured to utilize a metric to identify (e.g., classify) two or more patterns of analyte pattern analysis 1010 from each other. As shown in FIG. 10B, cluster analysis 1040 may include Y-axis (arb. units) 1042, X-axis (arb. units) 1044, first pattern 1046 (e.g., weekday, day shift, not traveling, etc.), and second pattern 1048 (e.g., weekend, night shift, traveling, etc.). Cluster analysis 1040 may group points of the point set from analyte pattern analysis 1010, each point representing one of the daily analyte profiles (e.g., glucose segment I 1016 and glucose segment J 1018), into first and second patterns 1046, 1048.

In some aspects, cluster analysis 1040 may include one or more algorithms to separate analyte pattern analysis 1010 into two or more patterns (e.g., first and second patterns 1046, 1048). For example, the one or more algorithms may include a centroid model (e.g., k-means clustering), a model-based clustering (e.g., a Gaussian mixture model), a connectivity model (e.g., hierarchical clustering), self-organizing mapping (e.g., unsupervised neural network), or any other suitable clustering algorithm. In some aspects, cluster analysis 1040 may utilize k-means clustering. In some aspects, cluster analysis 1040 may utilize a Gaussian mixture model.

In some aspects, as shown in FIG. 10B, cluster analysis 1040 may also consider user data 1020 (e.g., timing data, calendar data, user schedule data, user work data, travel data, GPS location data, time zone data, etc.) along with analyte pattern analysis 1010 to identify two or more patterns (e.g., first and second patterns 1046, 1048). In some aspects, for example, user data 1020 may be combined with and/or used to label corresponding glucose profiles of analyte pattern analysis 1010 to identify two or more patterns (e.g., first and second patterns 1046, 1048) with cluster analysis 1040.

In some aspects, cluster analysis 1040 may utilize a metric to identify (e.g., classify) the two or more patterns from each other (e.g., first and second patterns 1046, 1048). For example, the metric may include a distance reduction ratio, a Dunn index, a Davies-Bouldin index, a silhouette coefficient, a purity, a Rand index, an F-measure, a Jaccard index, a Dice index, a Fowlkes-Mallows index, a Chi index, a confusion matrix, a Hopkins statistic, or any other suitable metric. In some aspects, cluster analysis 1040 may utilize a distance reduction ratio. In some aspects, the metric may include a threshold above which the two or more patterns are identified (e.g., classified). For example, for the distance reduction ratio metric, the threshold may be at least 20%, at least 25%, or at least 30%. In some aspects, the threshold is in a range from about 20% to about 30%. In some aspects, the threshold is in a range from about 15% to about 40%.

In some aspects, first pattern 1046 may include weekdays. In some aspects, first pattern 1046 may include a day shift. In some aspects, first pattern 1046 may include not traveling. In some aspects, second pattern 1048 may include weekends. In some aspects, second pattern 1048 may include a night shift. In some aspects, second pattern 1048 may include traveling. In some aspects, first pattern 1046 may include weekdays and second pattern 1048 may include weekends. In some aspects, first pattern 1046 may include a day shift and second pattern 1048 may include a night shift. In some aspects, first pattern 1046 may include not traveling and second pattern 1048 may include traveling.

FIG. 10C shows first plot 1060 of first titration profile 1066, according to an example aspect. First titration profile 1066 may be configured to provide a personalized and optimized approach to adjusting insulin dosages for an individual based on the identified first pattern 1046 (e.g., weekday, day shift, not traveling, etc.) to achieve optimal glucose control. First titration profile 1066 may be further configured to increase or decrease an insulin dose to adjust or maintain glucose levels of the individual within an optimal target range (e.g., target glucose level within 90-110 mg/dL). In some aspects, model 1000 may generate first titration profile 1066 based on first pattern 1046 determined by cluster analysis 1040. In some aspects, for example, model 1000 may utilize one or more titration algorithms (e.g., fixed-dose titration, treat-to-target titration, carbohydrate-to-insulin ratio (CR) and correction factor (CF) titration, etc.), using the glycemic outcomes and insulin dosing regimen of first pattern 1046 as inputs, to generate first titration profile 1066.

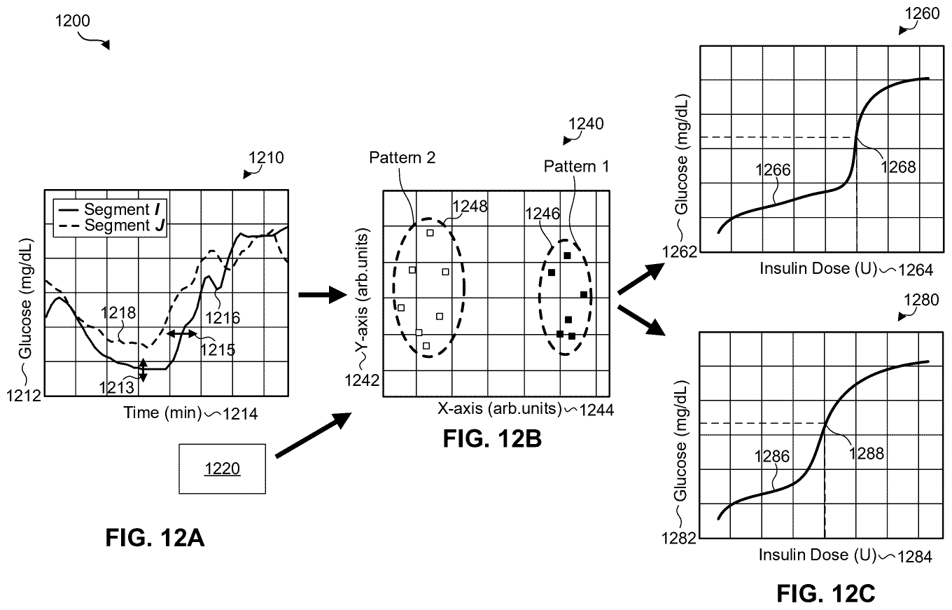

In some aspects, a titration profile (e.g., first titration profile 1066, second titration profile 1086, first titration profile 1266, second titration profile 1286) may be based on one or more input parameters. For example, the input parameters of the titration profile may include glucose level, dose amount, insulin sensitivity factor (ISF), carbohydrate-to-insulin ratio (CR), or a combination thereof. In some aspects, the titration profile (e.g., first titration profile 1066, second titration profile 1086, first titration profile 1266, second titration profile 1286) may generate one or more dose recommendations (e.g., meal dose, correction dose, basal dose, etc.) based on the one or more input parameters (e.g., dose amount, ISF, CR) particular to that titration profile, for example, the one or more input parameters that generate the titration profile (e.g., first titration profile 1066 and/or second titration profile 1086 shown in FIG. 10C, first titration profile 1266 and/or second titration profile 1286 shown in FIG. 12C). In some aspects, the one or more dose recommendations may be output to an insulin delivery device (e.g., smart insulin pen, smart insulin pen cap, pump, etc.). In some aspects, the one or more dose recommendations may be output to a user device (e.g., smart phone, reader, etc.). In some aspects, the one or more dose recommendations may be output to an HCP device for approval before sending to the user device.

In some aspects, a titration profile (e.g., first titration profile 1066, second titration profile 1086, first titration profile 1266, second titration profile 1286) may be based on a trivariate relationship between dose amount, ISF, and CR. In some aspects, for example, the titration profile may be related to an ellipsoid where the three perpendicular axes of symmetry of the ellipsoid represent dose amount, ISF, and CR of the titration profile. In some aspects, for example, the first semi-axis (a, X-axis) of the ellipsoid would represent dose amount (e.g., insulin dose amount), the second semi-axis (b, Y-axis) of the ellipsoid would represent ISF, and the third semi-axis (c, Z-axis) of the ellipsoid would represent CR. In some aspects, the titration profile may be defined by an ellipsoid, as represented by Equation (5) below:

$$\frac{{x}^{2}}{{( dose amount )}^{2}} + \frac{{y}^{2}}{{( ISF )}^{2}} + \frac{{Z}^{2}}{{( CR )}^{2}} = 1 \qquad ( 5 )$$

As shown in FIG. 10C, first plot 1060 shows glucose level (mg/dL) 1062 as a function of insulin dose (U) 1064 (1 U of insulin refers to 0.01 mL of insulin). In some aspects, insulin dose (U) 1064 may be for basal dose. In some aspects, insulin dose (U) 1064 may be for a bolus dose. First plot 1060 includes first titration profile 1066 corresponding to a graphical representation or curve of how glucose levels of an individual are affected by a change in insulin dose (U). In some aspects, as shown in FIG. 10C, first titration profile 1066 may include equivalence point 1068 corresponding to a point on the curve for optimal dosing (e.g., glucose level of 100 mg/dL for an insulin (e.g., basal) dose of 20 U).

FIG. 10C shows second plot 1080 of second titration profile 1086, according to an example aspect. Second titration profile 1086 may be configured to provide a personalized and optimized approach to adjusting insulin dosages for an individual based on the identified second pattern 1048 (e.g., weekend, night shift, traveling, etc.) to achieve optimal glucose control. Second titration profile 1086 may be further configured to increase or decrease an insulin dose to adjust or maintain glucose levels of the individual within an optimal target range (e.g., target glucose level within 90-110 mg/dL). In some aspects, model 1000 may generate second titration profile 1086 based on second pattern 1048 determined by cluster analysis 1040. In some aspects, for example, model 1000 may utilize one or more titration algorithms (e.g., fixed-dose titration, treat-to-target titration, CR and CF titration, etc.), using the glycemic outcomes and insulin dosing regimen of second pattern 1048 as inputs, to generate second titration profile 1086.

As shown in FIG. 10C, second plot 1080 shows glucose level (mg/dL) 1082 as a function of insulin dose (U) 1084 (1 U of insulin refers to 0.01 mL of insulin). Second plot 1080 includes second titration profile 1086 corresponding to a graphical representation or curve of how glucose levels of an individual are affected by a change in insulin dose (U). In some aspects, as shown in FIG. 10C, second titration profile 1086 may include equivalence point 1088 corresponding to a point on the curve for optimal dosing (e.g., glucose level of 100 mg/dL for an insulin (e.g., basal) dose of 30 U).

In some aspects, first titration profile 1066 may include weekdays. In some aspects, first titration profile 1066 may include a day shift. In some aspects, first titration profile 1066 may include not traveling. In some aspects, second titration profile 1086 may include weekends. In some aspects, second titration profile 1086 may include a night shift. In some aspects, second titration profile 1086 may include traveling. In some aspects, first titration profile 1066 may include weekdays and second titration profile 1086 may include weekends. In some aspects, first titration profile 1066 may include a day shift and second titration profile 1086 may include a night shift. In some aspects, first titration profile 1066 may include not traveling and second titration profile 1086 may include traveling.

In some aspects, the two or more patterns identified by cluster analysis 1040 may include weekdays and weekends, for example, first pattern 1046 may include weekdays and second pattern 1048 may include weekends. In some aspects, first titration profile 1066 may correspond to weekdays and second titration profile 1086 may correspond to weekends. In some aspects, as shown in FIG. 10C, second titration profile 1086 may have a higher insulin dose range (e.g., glucose level of 100 mg/dL for an insulin (e.g., basal) dose of 30 U at equivalence point 1088) than first titration profile 1066 (e.g., glucose level of 100 mg/dL for an insulin (e.g., basal) dose of 20 U at equivalence point 1068). In some aspects, the first titration profile 1066 may be for a basal insulin dose (e.g., glucose level of 100 mg/dL for a basal insulin dose of 20 U at equivalence point 1068). In some aspects, the first titration profile 1066 may be for a rapid-acting bolus dose (e.g., glucose level of 100 mg/dL for a bolus dose of 4 U at equivalence point 1068, for example, based on 1 U of rapid-acting insulin for every 15 grams of carbohydrates). In some aspects, the second titration profile 1086 may be for a basal insulin dose (e.g., glucose level of 100 mg/dL for a basal insulin dose of 30 U at equivalence point 1088). In some aspects, the second titration profile 1086 may be for a rapid-acting bolus dose (e.g., glucose level of 100 mg/dL for a bolus dose of 6 U at equivalence point 1088, for example, based on 1 U of rapid-acting insulin for every 15 grams of carbohydrates).

In some aspects, the two or more patterns identified by cluster analysis 1040 may include a day shift and a night shift, for example, first pattern 1046 may include a day shift and second pattern 1048 may include a night shift. In some aspects, first titration profile 1066 may correspond to a day shift and second titration profile 1086 may correspond to a night shift. In some aspects, second titration profile 1086 may be shifted later in time (e.g., by 6 to 12 hours) relative to first titration profile 1066.

In some aspects, the two or more patterns identified by cluster analysis 1040 may include not traveling and traveling, for example, first pattern 1046 may include not traveling and second pattern 1048 may include traveling. In some aspects, first titration profile 1066 may correspond to not traveling and second titration profile 1086 may correspond to traveling. In some aspects, second titration profile 1086 may be shifted in time (e.g., by one or more time zones) relative to first titration profile 1066. In some aspects, for example, second titration profile 1086 may be shifted in time relative to first titration profile 1066 based on a current location (e.g., GPS location) of the user.

In some aspects, model 1000 may further include dynamically updating a titration profile (e.g., first titration profile 1066, second titration profile 1086) in real time. In some aspects, for example, the titration profile (e.g., second titration profile 1086) may be continually updated in real time based on measured glucose data, medication doses, ISF, and/or CR. In some aspects, for example, the titration profile (e.g., second titration profile 1086) may be shifted or changed based on a change of one or more parameters of the titration profile (e.g., dose amount, ISF, CR). For example, equivalence point 1088 of second titration profile 1086 may be shifted (e.g., increased or decreased) based on the change of one or more parameters of second titration profile 1086 (e.g., glucose level, dose amount, ISF, CR). In some aspects, for example, a titration profile (e.g., first titration profile 1066, second titration profile 1086) may be updated based on a variability of the titration profile over time. In some aspects, for example, the variability may be based on a change of a glucose level, a change of a dose amount, a change of an ISF, and/or a change of a CR of the titration profile (e.g., first titration profile 1066, second titration profile 1086). In some aspects, for example, the variability may include a threshold above which the titration profile (e.g., second titration profile 1086) is updated based on an updated pattern (e.g., second pattern 1048) that includes the recent variability (e.g., a change of a glucose level, a change of a dose amount, a change of an ISF, and/or a change of a CR). In some aspects, for example, the threshold may be at least 20%, at least 25%, or at least 30%. In some aspects, the threshold is in a range from about 20% to about 30%. In some aspects, the threshold is in a range from about 15% to about 40%.

In some aspects, updating the titration profile based on a variability helps optimize and maintain optimal dose settings (e.g., fixed dose amounts) within an optimal target range of the user (e.g., glucose level of 100 mg/dL), for example, based on changes to one or more parameters of the titration profile over time (e.g., glucose level, dose amount, ISF, and/or CR). In some aspects, once optimal settings are reached, the titration profile may oscillate (e.g., increase or decrease) around the optimal settings, and a variability (e.g., change in dose amount, ISF, and/or CR) may be used to fine tune the titration profile within an optimal target range, for example, glucose level within ±2% of 100 mg/dL.

In some aspects, model 1000 may further include generating a new titration profile when a variability (e.g., change in glucose level, dose amount, ISF, and/or CR) of one of the titration profiles is above a threshold. In some aspects, the threshold is in a range from about 20% to about 30%. In some aspects, the threshold is in a range from about 15% to about 40%. In some aspects, model 1000 may automatically generate the new titration profile when the threshold is exceeded. In some aspects, model 1000 may send a communication to the user to confirm the new titration profile should be generated and/or incorporated into the therapy regimen upon user confirmation.

In some aspects, model 1000 may include a machine learning model. In some aspects, for example, analyte pattern analysis 1010, cluster analysis 1040, and generation of first and second titration profiles 1066, 1086 may be performed by a machine learning model. In some aspects, the machine learning model may include an unsupervised machine learning model performing cluster analysis.

In some aspects, analyte pattern analysis 1010, cluster analysis 1040, and generation of first and second titration profiles 1066, 1086 of model 1000 may be performed by a generative AI model. In some aspects, model 1000 may include prompting the generative AI model to determine whether the plurality of analyte profiles (e.g., glucose segment I 1016 and glucose segment J 1018) of analyte pattern analysis 1010 are correlated to weekdays or weekends. In some aspects, model 1000 may include prompting the generative AI model to determine whether the plurality of analyte profiles (e.g., glucose segment I 1016 and glucose segment J 1018) of analyte pattern analysis 1010 are correlated to a day shift or a night shift. In some aspects, model 1000 may include prompting the generative AI model to determine whether the plurality of analyte profiles (e.g., glucose segment I 1016 and glucose segment J 1018) of analyte pattern analysis 1010 are correlated to not traveling or traveling.

In some aspects, model 1000 may include prompting the generative AI model to determine whether the plurality of analyte profiles (e.g., glucose segment I 1016 and glucose segment J 1018) of analyte pattern analysis 1010 are correlated to a first type of user data (e.g., weekdays, weekends, day shift, night shift, not traveling, traveling, etc.). In some aspects, model 1000 may include prompting the generative AI model to determine whether the two or more patterns (e.g., first and second patterns 1046, 1048) are correlated to a second type of user data. For example, model 1000 may determine whether first pattern 1046 (e.g., weekdays) and second pattern 1048 (e.g., weekends) are correlated to a second type of user data (e.g., not traveling on weekdays, traveling on weekends, day shift on weekdays, night shift on weekends, etc.).

In some aspects, analyte pattern analysis 1010, cluster analysis 1040, and generation of first and second titration profiles 1066, 1086 of model 1000 may be performed by a LLM (e.g., GPT, Claude, Gemini, Copilot, DeepSeek, etc.) that compares the two or more identified patterns (e.g., first and second patterns 1046, 1048) to a second type of user data to determine a level of correlation. For example, model 1000 may utilize the LLM to compare first pattern 1046 (e.g., weekdays) and second pattern 1048 (e.g., weekends) to a second type of user data (e.g., not traveling on weekdays, traveling on weekends, day shift on weekdays, night shift on weekends, etc.) and determine the level of correlation.

In some aspects, model 1000 may utilize the LLM to generate or update first titration profile 1066 based on the level of correlation between first pattern 1046 (e.g., weekdays) and the second type of user data (e.g., not traveling on weekdays, day shift on weekdays, etc.). In some aspects, for example, model 1000 may utilize the LLM to implement one or more titration algorithms (e.g., fixed-dose titration, treat-to-target titration, CR and CF titration, etc.), using the glycemic outcomes and insulin dosing regimen of first pattern 1046 as inputs, to generate first titration profile 1066. In some aspects, for example, model 1000 may utilize the LLM to implement one or more titration algorithms (e.g., fixed-dose titration, treat-to-target titration, CR and CF titration, etc.), using the glycemic outcomes and insulin dosing regimen of first pattern 1046 and the level of correlation of first pattern 1046 (e.g., weekdays) to the second type of user data (e.g., not traveling, day shift, etc.) as inputs, to generate or update first titration profile 1066.

In some aspects, model 1000 may utilize the LLM to generate or update second titration profile 1086 based on the level of correlation between second pattern 1048 (e.g., weekends) and the second type of user data (e.g., traveling on weekends, night shift on weekends, etc.). In some aspects, for example, model 1000 may utilize the LLM to implement one or more titration algorithms (e.g., fixed-dose titration, treat-to-target titration, CR and CF titration, etc.), using the glycemic outcomes and insulin dosing regimen of second pattern 1048 as inputs, to generate second titration profile 1086. In some aspects, for example, model 1000 may utilize the LLM to implement one or more titration algorithms (e.g., fixed-dose titration, treat-to-target titration, CR and CF titration, etc.), using the glycemic outcomes and insulin dosing regimen of second pattern 1048 and the level of correlation of second pattern 1046 (e.g., weekends) to the second type of user data (e.g., traveling, night shift, etc.) as inputs, to generate or update second titration profile 1086.

### Example Flow Diagram for Titration Profiles

FIG. 11 illustrates flow diagram 1100, according to an example aspect. For example, flow diagram 1100 may be for analyte pattern analysis system 400 shown in FIG. 4. For example, flow diagram 1100 may be for software application 470 shown in FIG. 4. Flow diagram 1100 may be configured to identify analyte patterns (e.g., glucose patterns). Flow diagram 1100 may be further configured to analyze analyte profiles of analyte data of a user and group the analyte patterns into two or more patterns. Flow diagram 1100 may be further configured to develop personalized and optimized titration profiles for adaptive dose guidance for an individual. Flow diagram 1100 may be further configured to identify one or more patterns (e.g., weekdays vs. weekends, day shift vs. night shift, not traveling vs. traveling, etc.) and generate one or more titration profiles for each of the identified patterns. Flow diagram 1100 may be further configured to streamline analyte pattern analysis (e.g., glucose pattern analysis) and generation of titration profiles using a model (e.g., model 1000, generative AI model, LLM).

It is to be appreciated that not all operations in FIG. 11 are needed to perform the disclosure provided herein. Further, some of the operations may be performed simultaneously, sequentially, and/or in a different order than shown in FIG. 11. Flow diagram 1100 shall be described with reference to FIGS. 4-9 and 10A-10C. However, flow diagram 1100 is not limited to those example aspects. Although flow diagram 1100 is shown in FIG. 11 as a stand-alone method, the aspects of this disclosure may be used with other apparatuses, systems, and/or methods, such as, but not limited to, elements in FIGS. 1-9, 10A-10C, 12A-12C, and 13-15, e.g., analyte monitoring system 100, sensor control device 102, receiver device 200, analyte pattern analysis system 400, flow diagram 500, machine learning model 600, cluster plot 700, calendar plot 800, distance plot 900, model 1000, model 1200, flow diagram 1300, flow diagram 1400, and/or flow diagram 1500. In some aspects, flow diagram 1100 may be implemented by analyte pattern analysis system 400 shown in FIG. 4 (e.g., via a processor or software application 470). In some aspects, flow diagram 1100 may be implemented by software application 470 shown in FIG. 4. In some aspects, flow diagram 1100 may be implemented by model 1000 shown in FIGS. 10A-10C.

In operation 1102, as shown in the example of FIGS. 4-9 and 10A-10C, analyte levels of a user may be monitored by an analyte monitoring device (e.g., OBU 410 shown in FIG. 4). For example, daily glucose profiles of the user may be monitored.

In operation 1104, as shown in the example of FIGS. 4-9 and 10A-10C, user data of the user may be received by at least one processor (e.g., receiver device 450 running software application 470 shown in FIG. 4), including analyte data monitored by the analyte monitoring device. For example, the user data may include analyte data of the user, for example, glucose data (e.g., daily glucose profiles).

In some aspects, the user data may include medication dosing data (e.g., via pen cap 422 shown in FIG. 4). In some aspects, for example, pen cap 422 may detect a dosing event of injection pen 426 (e.g., insulin, GLP-1, etc.), for example, the dosing event may be inferred from a decapping event of pen cap 422 from injection pen 426 and a capping event of pen cap 422 to injection pen 426. In some aspects, the user data may include meal data (e.g., via meal logs 474 shown in FIG. 4). In some aspects, for example, meal logs 474 and/or one or more smart devices (e.g., first smart device 430 and/or second smart device 440 shown in FIG. 4) may detect a meal event of the user (e.g., breakfast, lunch, dinner, snack). In some aspects, the user data may include activity data, exercise data, stress data, sleep data, location data, travel data, calendar data, or a combination thereof (e.g., via activity logs 476 shown in FIG. 4). In some aspects, for example, activity logs 476 and/or one or more smart devices (e.g., first smart device 430, second smart device 440, and/or receiver device 450 shown in FIG. 4) may detect an activity event of the user, an exercise event of the user, a stress event of the user, a sleep event of the user, a location event of the user, a travel event of the user, or a combination thereof.

In some aspects, the user data may include timing data (e.g., calendar data) of the analyte data and the medication dosing data of the user. For example, the user data may distinguish between analyte data and medication dosing data collected on weekdays from analyte data and medication dosing data collected on weekends. In some aspects, the user data may include activity data (e.g., day shift vs. night shift) of the user. For example, the user data may distinguish between hours and/or days when the user is working on a day shift from hours and/or days when the user is working on a night shift. In some aspects, for example, activity logs 476 and/or one or more smart devices (e.g., first smart device 430, second smart device 440, and/or receiver device 450 shown in FIG. 4) may detect a day shift or a night shift of the user (e.g., a work log). In some aspects, the user data may include travel data (e.g., not traveling vs. traveling) of the user. For example, the user data may distinguish between a current location (e.g., GPS location, time zone) when the user not traveling from a current location when the user is traveling. In some aspects, for example, activity logs 476 and/or one or more smart devices (e.g., first smart device 430, second smart device 440, and/or receiver device 450 shown in FIG. 4) may detect a current location or time zone (e.g., GPS location) of the user (e.g., a travel log).

In operation 1106, as shown in the example of FIGS. 4-9 and 10A-10C, analyte profiles (e.g., glucose segment I 1016 and glucose segment J 1018 shown in FIG. 10A) of the analyte data over an analysis period (e.g., 14 days) may be analyzed by a model (e.g., model 1000 shown in FIGS. 10A-10C). For example, the analyte profiles may include daily glucose profiles.

In some aspects, the model may receive the analyte profiles (e.g., daily glucose profiles) and group them into patterns. The grouping may be performed based on one or more metrics for each analyte profile (e.g., daily glucose profile). In some aspects, the one or more metrics may include a similar mean glucose. In some aspects, the one or more metrics may include a similar glucose variability. In some aspects, the one or more metrics may include a minimum peak glucose level. In some aspects, the model may include assessing a distance between the daily glucose profiles. For example, the distance may be based on a MARD (e.g., Equation 3 above). In some aspects, first and second daily glucose profiles (e.g., glucose segment J 1018 shown in FIG. 10A) may be grouped into a first pattern type (e.g., first pattern 1046) when the distance is at or below a threshold. For example, the threshold may be based on a predetermined analyte value (e.g., glucose level of 30 mg/dL, glucose difference of 30 mg/dL). In some aspects, the model may include unsupervised machine learning (e.g., no training). In some aspects, the model may include supervised machine learning (e.g., with training). In some aspects, the model may include a generative AI model (e.g., LLM).

In some aspects, the model may also receive user data (e.g., user data 1020 shown in FIG. 10A). In some aspects, the model may utilize the analyte profiles along with the user data (e.g., weekdays, weekends, day shift, night shift, traveling, not traveling, etc.) for pattern identification in the cluster analysis. In some aspects, the user data (e.g., user data 1020) may include timing data, calendar data, user schedule data, user work data, travel data, GPS location data, time zone data, or a combination thereof. In some aspects, for example, the user data may include timing data or calendar data to determine whether the glucose profiles occur on the weekdays or on the weekends to be grouped into patterns. In some aspects, for example, the user data may include user schedule data or user work data to determine whether the glucose profiles occur during a day shift or a night shift of the user to be grouped into patterns. In some aspects, for example, the user data may include user travel data, GPS location data, or time zone data to determine whether the glucose profiles occur when the user is traveling or not traveling to be grouped into patterns.

In operation 1108, as shown in the example of FIGS. 4-9 and 10A-10C, the daily analyte profiles may be grouped into two or more patterns (e.g., first and second patterns 1046, 1048 shown in FIG. 10B) based on the analysis performed by the model (e.g., model 1000 shown in FIGS. 10A-10C).

In some aspects, the grouping may be limited to no greater than three patterns. For example, when the analysis period is in a range from 3 days to 14 days, the grouping is limited to no more than three patterns (e.g., minimum data points for cluster analysis). In some aspects, limiting to no greater than three patterns improves efficiency and helps streamline analysis by limiting complexity and ensuring there is sufficient data to resolve unique patterns (e.g., weekdays, day shift, traveling, etc.). In some aspects, the grouping may include a hierarchy for selecting the patterns, such that user health concerns (e.g., illness, night shift, traveling, etc.) are given priority over more typical or standard patterns (e.g., not ill, day shift, not traveling, etc.).

In operation 1110, as shown in the example of FIGS. 4-9 and 10A-10C, titration profiles (e.g., first and second titration profiles 1066, 1086 shown in FIG. 10C) each corresponding to one of the two or more patterns (e.g., first and second patterns 1046, 1048 shown in FIG. 10B) identified may be generated by the model (e.g., model 1000 shown in FIGS. 10A-10C).

In some aspects, the model may generate first titration profile 1066 based on first pattern 1046 determined by cluster analysis 1040. In some aspects, for example, model 1000 may utilize one or more titration algorithms (e.g., fixed-dose titration, treat-to-target titration, CR and CF titration, etc.), using the glycemic outcomes and insulin dosing regimen of first pattern 1046 as inputs, to generate first titration profile 1066. In some aspects, the model may generate second titration profile 1086 based on second pattern 1048 determined by cluster analysis 1040. In some aspects, for example, model 1000 may utilize one or more titration algorithms (e.g., fixed-dose titration, treat-to-target titration, CR and CF titration, etc.), using the glycemic outcomes and insulin dosing regimen of second pattern 1048 as inputs, to generate second titration profile 1086.

In some aspects, for example, the model may utilize a LLM to implement one or more titration algorithms (e.g., fixed-dose titration, treat-to-target titration, CR and CF titration, etc.), using the glycemic outcomes and insulin dosing regimen of first pattern 1046 and/or second pattern 1048 as inputs, to generate first titration profile 1066 and/or second titration profile 1086. In some aspects, for example, the model may utilize the LLM to implement one or more titration algorithms (e.g., fixed-dose titration, treat-to-target titration, CR and CF titration, etc.), using the glycemic outcomes and insulin dosing regimen of first pattern 1046 and a level of correlation of first pattern 1046 (e.g., weekdays) to a second type of user data (e.g., not traveling, day shift, etc.) as inputs, to generate or update first titration profile 1066. In some aspects, for example, the model may utilize the LLM to implement one or more titration algorithms (e.g., fixed-dose titration, treat-to-target titration, CR and CF titration, etc.). In some aspects, for example, the LLM may use the glycemic outcomes and insulin dosing regimen of an identified pattern (e.g., second pattern 1048) and a level of correlation of the identified pattern (e.g., second pattern 1048 identifying weekdays) to a second type of user data (e.g., not traveling, day shift, etc.) as inputs, to generate or update the titration profile (e.g., second titration profile 1086). In some aspects, for example, the LLM may generate or update the titration profile (e.g., second titration profile 1086) by changing one or more parameters of the titration profile (e.g., dose amount, ISF, and/or CR) based on the level of correlation.

In operation 1112, optionally, as shown in the example of FIGS. 4-9 and 10A-10C, one or more of the titration profiles (e.g., first and second titration profiles 1066, 1086 shown in FIG. 10C) may be dynamically updated in real time by the model based on a variability of a specific titration profile over time.

In some aspects, the model may include dynamically updating a titration profile (e.g., first titration profile 1066, second titration profile 1086) in real time. In some aspects, for example, the titration profile (e.g., first titration profile 1066, second titration profile 1086) may be continually updated in real time based on measured glucose data, medication doses, ISF, and/or CR. In some aspects, for example, the titration profile (e.g., first titration profile 1066, second titration profile 1086) may be shifted or changed based on a change of one or more parameters of the titration profile (e.g., dose amount, ISF, CR). For example, an equivalence point (e.g., equivalence point 1068 of first titration profile 1066, equivalence point 1088 of second titration profile 1086) may be shifted (e.g., increased or decreased) based on the change of one or more parameters of the titration profile (e.g., dose amount, ISF, CR).

In some aspects, for example, a titration profile (e.g., first titration profile 1066, second titration profile 1086) may be updated based on a variability of the titration profile over time. In some aspects, for example, the variability may be based on a change of a glucose level, a change of a dose amount, a change of an ISF, and/or a change of a CR of the titration profile (e.g., first titration profile 1066, second titration profile 1086). In some aspects, for example, the variability may include a threshold above which the titration profile (e.g., first titration profile 1066, second titration profile 1086) is updated based on an updated pattern (e.g., second pattern 1048) that includes the recent variability (e.g., a change of a glucose level, a change of a dose amount, a change of an ISF, and/or a change of a CR). In some aspects, for example, the threshold may be at least 20%, at least 25%, or at least 30%. In some aspects, the threshold is in a range from about 20% to about 30%. In some aspects, the threshold is in a range from about 15% to about 40%.

In some aspects, the model may further include generating a new titration profile when a variability of one of the titration profiles is above a threshold. In some aspects, the threshold is in a range from about 20% to about 30%. In some aspects, the threshold is in a range from about 15% to about 40%. In some aspects, the model may automatically generate the new titration profile when the threshold is exceeded. In some aspects, the model may send a communication (e.g., output an alert) to the user to confirm the new titration profile should be generated and/or incorporated into the therapy regimen upon user confirmation. In some aspects, the user may be a PwD. In some aspects, the user may be a HCP.

### Example Model for Specific Titration Profiles

As discussed above, a problem in the art is that glucose levels are affected by multiple factors dependent upon an individual's daily routine, profession, activities, status, and self-care (e.g., medication dosing, meals, exercise, stress, hormones). Also, current daily monitoring schemes (e.g., AGP) do not correlate day-to-day analyte pattern variations with specific user data (e.g., pregnancy, menstruation, illness, etc.) to identify distinct user patterns over time. Further, current daily monitoring schemes do not consider specific user data to identify distinct patterns and generate specific titration profiles based on those identified patterns (e.g., pregnant vs. not pregnant, menstruating vs. not menstruating, ill vs. not ill, etc.).

In order to address this problem, the systems and methods of the present disclosure identify two or more patterns associated with an individual's multi-day analyte data (e.g., glucose data) and specific user data (e.g., pregnancy, menstruation, illness, etc.), for example, either provided by the user or determined by one or more monitoring devices, and generate improved titration profiles for each of the identified patterns. This provides an improvement to diabetes management technology by correlating the analyte data to a specific state or status of an individual and optimizing a titration profile for that specific pattern. For example, the system may identify a first analyte pattern (e.g., pregnant, menstruating, ill, etc.) distinguishable from a second analyte pattern (e.g., not pregnant, not menstruating, not ill, etc.), and then generate a corresponding first titration profile based on the first analyte pattern and a second titration profile based on the second analyte pattern. Further, the present disclosure may dynamically update a specific titration profile (e.g., pregnant, menstruating, ill, etc.) in real time based on a variability of that titration profile over time, which improves diabetes management technology by continually adjusting and maintaining that specific titration profile within an optimal target range over time.

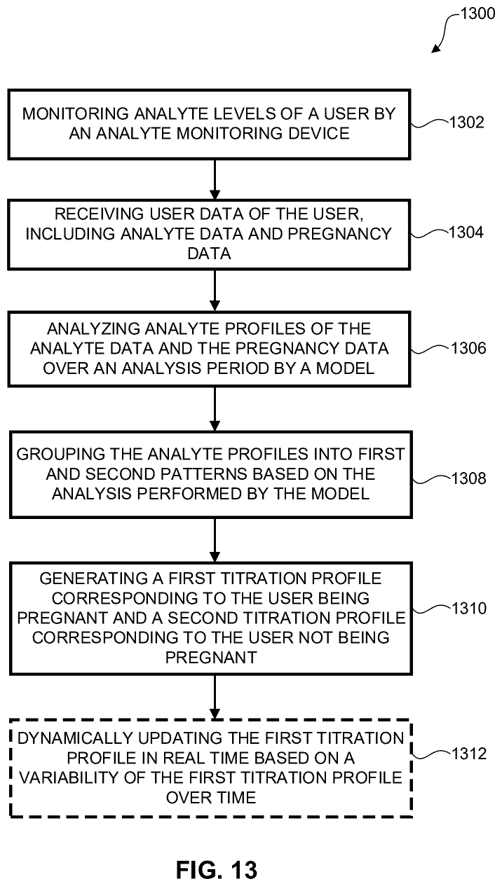

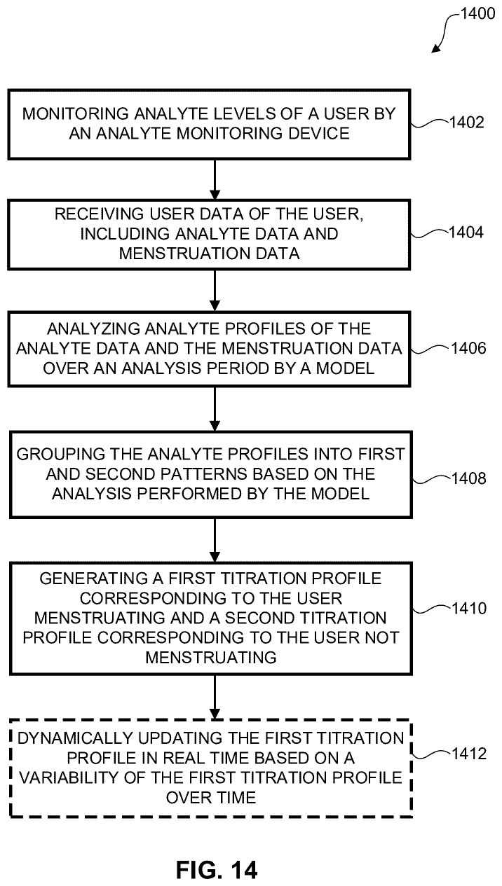

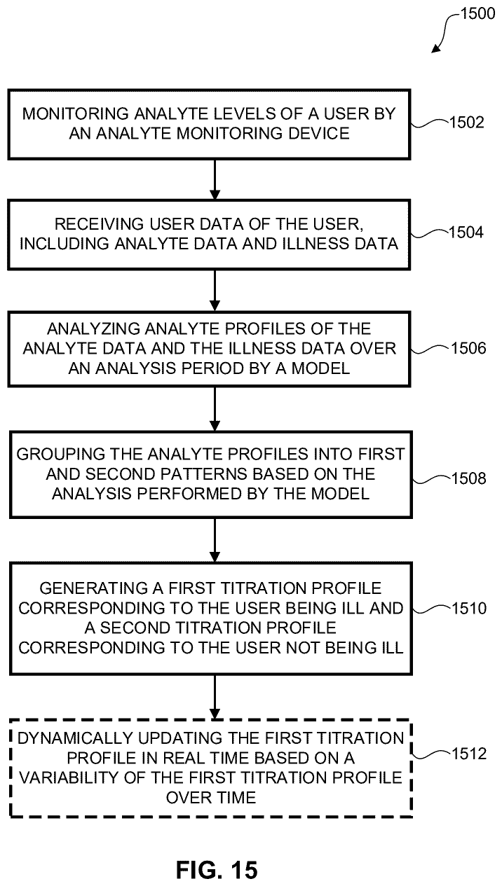

FIGS. 12A-12C illustrate model 1200, according to an example aspect. For example, model 1200 may be for software application 470 of analyte pattern analysis system 400 shown in FIG. 4. For example, model 1200 may be for flow diagram 1300 shown in FIG. 13 (e.g., pregnancy). For example, model 1200 may be for flow diagram 1400 shown in FIG. 14 (e.g., menstruation). For example, model 1200 may be for flow diagram 1500 shown in FIG. 15 (e.g., illness). Model 1200 may be configured to identify analyte patterns. Model 1200 may be further configured to analyze analyte profiles of analyte data of a user and specific user data (e.g., pregnancy, menstruation, illness, etc.), and group the analyte patterns into two or more patterns. Model 1200 may be further configured to develop personalized and optimized titration profiles for adaptive dose guidance for an individual. Model 1200 may be further configured to identify one or more patterns (e.g., pregnant vs. not pregnant, menstruating vs. not menstruating, ill vs. not ill, etc.) and generate one or more titration profiles for each of the identified patterns. Model 1200 may be further configured to utilize a generative AI model to determine whether analyte patterns are correlated to a specific user data (e.g., pregnant, not pregnant, menstruating, not menstruating, ill, not ill, etc.). Model 1200 may be further configured to utilize a LLM to compare identified analyte patterns to one or more types of user data to determine a level of correlation. Model 1200 may be further configured to streamline analyte pattern analysis using one or more algorithms (e.g., unsupervised machine learning, LLM).

It is to be appreciated that not all operations in FIGS. 12A-12C are needed to perform the disclosure provided herein. Further, some of the operations may be performed simultaneously, sequentially, and/or in a different order than shown in FIGS. 12A-12C. Model 1200 shall be described with reference to FIGS. 4-11 and 13-15. However, model 1200 is not limited to those example aspects. Although model 1200 is shown in FIGS. 12A-12C as a stand-alone model and/or process, the aspects of this disclosure may be used with other apparatuses, systems, and/or methods, such as, but not limited to, elements in FIGS. 1-11 and 13-15, e.g., analyte monitoring system 100, sensor control device 102, receiver device 200, analyte pattern analysis system 400, flow diagram 500, machine learning model 600, cluster plot 700, calendar plot 800, distance plot 900, model 1000, flow diagram 1100, flow diagram 1300, flow diagram 1400, and/or flow diagram 1500. In some aspects, model 1200 may be implemented by analyte pattern analysis system 400 shown in FIG. 4 (e.g., via a processor or software application 470). In some aspects, model 1200 may be implemented in flow diagram 1300 shown in FIG. 13 (e.g., via a processor or app). In some aspects, model 1200 may be implemented in flow diagram 1400 shown in FIG. 14 (e.g., via a processor or app). In some aspects, model 1200 may be implemented in flow diagram 1500 shown in FIG. 15 (e.g., via a processor or app).

The aspects of model 1000 shown in FIGS. 10A-10C, for example, and the aspects of model 1200 shown in FIGS. 12A-12C may be similar. Similar reference numbers are used to indicate features of the aspects of model 1000 shown in FIGS. 10A-10C and the similar features of the aspects of model 1200 shown in FIGS. 12A-12C. One difference between the aspects of model 1000 shown in FIGS. 10A-10C and the aspects of model 1200 shown in FIGS. 12A-12C is that model 1200 also considers specific user data 1220 (e.g., pregnancy data, menstruation data, illness data), user data that is not easily detected from monitored data and may require user confirmation, with analyte pattern analysis 1210 to determine first and second patterns 1246, 1248 via cluster analysis 1240 and generate first titration profile 1266 and second titration profile 1286 from cluster analysis 1240 of first pattern 1246 and second pattern 1248, respectively, rather than utilizing analyte pattern analysis 1010 and user data 1020 (e.g., may be determined from one or more monitoring devices) to determine first and second patterns 1046, 1048 via cluster analysis 1040 of model 1000 shown in FIGS. 10A-10C.

Model 1200 is similar to model 1000 shown in FIGS. 10A-10C and similar reference numbers are used to indicate the similar features of model 1000 shown in FIGS. 10A-10C and model 1200 shown in FIGS. 12A-12C. Discussion of model 1200 components, processes, properties, and/or functionality (e.g., analyte pattern analysis 1210, cluster analysis 1240, first titration profile 1266, second titration profile 1286) is not duplicated here for brevity, but the aspects and features of each are similar to model 1000 shown in FIGS. 10A-10C described above.

As shown in FIGS. 12A-12C, model 1200 may include analyte pattern analysis 1210, cluster analysis 1240, and first and second titration profiles 1266, 1286. In some aspects, the various components of model 1200, and the one or more software modules that make them up (e.g., receiver device 450, software application 470, remote server 480, etc.), may be implemented on a single processor, multiple processors, a single server, multiple web-servers, and/or intranet servers.

Analyte pattern analysis 1210 may be configured to analyze (e.g., compare) pairs of analyte profiles. For example, analyte pattern analysis 1210 may analyze pairs of daily glucose profiles. Analyte pattern analysis 1210 may be further configured to calculate a distance (e.g., MARD) between each pair of analyte profiles (e.g., each pair of daily glucose profiles). In some aspects, analyte pattern analysis 1210 may be further configured to generate a distance matrix (e.g., similar to distance matrix 620 shown in FIG. 6B) based on a distance between each pair of analyte profiles. In some aspects, analyte pattern analysis 1210 may be further configured to generate a point set (e.g., similar to point set 630 shown in FIG. 6C) constructed from the distance matrix (e.g., similar to distance matrix 620 shown in FIG. 6B) by multidimensional scaling. In some aspects, analyte pattern analysis 1210 may be further configured generate a point set (e.g., similar to point set 630 shown in FIG. 6C) to represent (e.g., a lower-dimensional representation) elements of the distance matrix (e.g., similar to distance Dᵢⱼ 626 shown in FIG. 6B) as a set of points in a plane (e.g., a scatter plot). In some aspects, analyte pattern analysis 1210 may be further configured to provide the point set (e.g., similar to point set 630 shown in FIG. 6C) to cluster analysis 1240 to group points of the point set into two or more patterns.

As shown in FIG. 12A, analyte pattern analysis 1210 may include glucose level (mg/dL) 1212 as a function of time (min) 1214 for pairs of analyte profiles of analyte data (e.g., daily glucose profiles of glucose data) of a user over an analysis period (e.g., monitored by OBU 410), for example, glucose segment I 1216 (e.g., indicated by a solid line) and glucose segment J 1218 (e.g., indicated by a dashed line). In some aspects, glucose level (mg/dL) 1212 may include a glucose difference between pairs of analyte profiles of analyte data (e.g., daily glucose profiles of glucose data).

In some aspects, as shown in FIG. 12A, analyte pattern analysis 1210 may include a glucose threshold Δg 1213, which represents an allowable spread or dispersion in glucose level from the daily glucose profiles (e.g., glucose segment I 1216). In some aspects, for example, glucose threshold Δg 1213 may be adjusted to a specified value (e.g., Δg=0%, ±1%, ±2%, ±5%, ±10%, etc. from the baseline glucose value of the corresponding daily glucose profile). In some aspects, as shown in FIG. 12A, analyte pattern analysis 1210 may include a time threshold Δt 1215, which represents an allowable spread or dispersion in time from the daily glucose profiles (e.g., glucose segment I 1216). In some aspects, for example, time threshold Δt 1215 may be adjusted to a specified value (e.g., Δt=0%, ±1%, ±2%, ±5%, ±10%, etc. from the baseline time value of the corresponding daily glucose profile).

As shown in FIG. 12A, user data 1220 (e.g., pregnancy data, menstruation data, illness data) may be received (e.g., via a user query) and/or determined (e.g., via one or more monitoring devices) and also used in cluster analysis 1240. In some aspects, model 1200 may include outputting a query to the user requesting user data 1220 (e.g., via receiver device 450 shown in FIG. 4). In some aspects, user data 1220 may be received from the user based on a response to the query. In some aspects, user data 1220 may be determined via one or more monitoring devices (e.g., first smart device 430, second smart device 440, and/or receiver device 450 shown in FIG. 4). In some aspects, user data 1220 may be received from a HCP. User data 1220 may be received from an electronic medical record (EMR).

In some aspects, for example, the query may be regarding pregnancy data (e.g., “Are you pregnant or not pregnant?”, “How far along is the pregnancy?”, “What is your current total body weight?”, “How many fetuses are developing?”, “Is your insulin resistance increasing?”, “What is your current ISF?”, etc.). In some aspects, for example, the query may be regarding menstruation data (e.g., “Are you menstruating or not menstruating?”, “What phase of the menstrual cycle are you experiencing?”, “Have you been diagnosed with type 1 diabetes?”, “Is your insulin resistance increasing?”, “What is your current ISF?”, etc.). In some aspects, for example, the query may be regarding illness data (e.g., “Are you ill or not ill?”, “How long have you been ill?”, “What type of illness?”, “Do you have multiple illnesses?”, “Are you receiving any medication or treatment for the illness?”, “Is it difficult to retain food?”, “Is your insulin resistance increasing?”, “Is your insulin production decreasing?”, “What is your current ISF?”, etc.).

In some aspects, user data 1220 may include pregnancy data regarding whether the user is pregnant or not pregnant. In some aspects, for example, pregnancy data may include how long the user has been pregnant (e.g., first trimester (1 to 13 weeks), second trimester (14 to 27 weeks), third trimester (28 to 40 weeks)). In some aspects, for example, pregnancy data may include how many fetuses are developing during the pregnancy (e.g., twin pregnancy, triplets, etc.). In some aspects, pregnancy data may include a change (e.g. an increase) in total body weight. In some aspects, pregnancy data may indicate whether the user has a decreased insulin sensitivity (e.g., ISF).

In some aspects, user data 1220 may include menstruation data regarding whether the user is menstruating or not menstruating. In some aspects, for example, menstruation data may include what phase of the menstrual cycle the user is experiencing (e.g., menstrual phase, follicular phase, ovulation phase, luteal phase). In some aspects, menstruation data may indicate whether the user is experiencing increased insulin resistance. In some aspects, for example, menstruation data may indicate whether the user has a decreased insulin sensitivity (e.g., ISF), for example, during the luteal phase.

In some aspects, user data 1220 may include illness data regarding whether the user is ill or not ill. In some aspects, for example, illness data may indicate the type of illness. In some aspects, for example, illness data may include whether there are multiple illnesses and what those illnesses are. In some aspects, illness data may indicate whether the user is receiving any medication and/or treatment for the illness (e.g., GLP-1, antibiotics, anti-inflammatory, radiotherapy, chemotherapy, etc.). In some aspects, illness data may indicate that it is difficult for the user to retain food (e.g., vomiting, abdominal pain, etc.). In some aspects, illness data may indicate whether the user is experiencing increased insulin resistance. In some aspects, illness data may indicate whether the user is experiencing decreased insulin production.

Cluster analysis 1240 may be configured to group points of a point set (e.g., similar to point set 630 shown in FIG. 6C) generated by analyte pattern analysis 1210 and user data 1220 into two or more patterns, such that the points (e.g., daily glucose profiles) of each pattern are more similar to each other than to those in other patterns. Cluster analysis 1240 may be further configured to utilize a metric to identify (e.g., classify) two or more patterns of analyte pattern analysis 1210 from each other. As shown in FIG. 12B, cluster analysis 1240 may include Y-axis (arb. units) 1242, X-axis (arb. units) 1244, first pattern 1246 (e.g., pregnant, menstruating, ill, etc.), and second pattern 1248 (e.g., not pregnant, not menstruating, not ill, etc.). Cluster analysis 1240 may group points of the point set from analyte pattern analysis 1210 and user data 1220, each point representing one of the daily analyte profiles (e.g., glucose segment I 1216 and glucose segment J 1218), into first and second patterns 1246, 1248.

In some aspects, as shown in FIG. 12B, cluster analysis 1240 may also consider user data 1220 along with analyte pattern analysis 1210 to identify two or more patterns (e.g., first and second patterns 1246, 1248). In some aspects, points of a point set (e.g., similar to point set 630 shown in FIG. 6C) generated by analyte pattern analysis 1210 may be labeled by user data 1220 (e.g., pregnant, not pregnant, menstruating, not menstruating, ill, not ill, etc.), and grouped accordingly into two or more patterns (e.g., first and second patterns 1046, 1048). In some aspects, cluster analysis 1240 may include one or more algorithms to separate analyte pattern analysis 1210 and user data 1220 into two or more patterns (e.g., first and second patterns 1046, 1048). For example, the one or more algorithms may include a centroid model (e.g., k-means clustering), a model-based clustering (e.g., a Gaussian mixture model), a connectivity model (e.g., hierarchical clustering), self-organizing mapping (e.g., unsupervised neural network), or any other suitable clustering algorithm. In some aspects, cluster analysis 1240 may utilize k-means clustering. In some aspects, cluster analysis 1240 may utilize a Gaussian mixture model.

In some aspects, cluster analysis 1240 may utilize a metric to identify (e.g., classify) the two or more patterns from each other (e.g., first and second patterns 1246, 1248) based on analyte pattern analysis 1210 and user data 1220. For example, the metric may include a distance reduction ratio, a Dunn index, a Davies-Bouldin index, a silhouette coefficient, a purity, a Rand index, an F-measure, a Jaccard index, a Dice index, a Fowlkes-Mallows index, a Chi index, a confusion matrix, a Hopkins statistic, or any other suitable metric. In some aspects, cluster analysis 1240 may utilize a distance reduction ratio. In some aspects, the metric may include a threshold above which the two or more patterns are identified (e.g., classified). For example, for the distance reduction ratio metric, the threshold may be at least 20%, at least 25%, or at least 30%. In some aspects, the threshold is in a range from about 20% to about 30%. In some aspects, the threshold is in a range from about 15% to about 40%.

In some aspects, first pattern 1246 may include a pregnancy pattern. In some aspects, first pattern 1246 may include a menstruation pattern. In some aspects, first pattern 1246 may include an illness pattern. In some aspects, second pattern 1248 may include a not pregnant pattern. In some aspects, second pattern 1248 may include a not menstruating pattern. In some aspects, second pattern 1248 may include a not ill pattern. In some aspects, first pattern 1246 may include a pregnancy pattern and second pattern 1248 may include a not pregnant pattern. In some aspects, first pattern 1246 may include a menstruation pattern and second pattern 1248 may include a not menstruating pattern. In some aspects, first pattern 1246 may include an illness pattern and second pattern 1248 may include a not ill pattern.

FIG. 12C shows first plot 1260 of first titration profile 1266, according to an example aspect. First titration profile 1266 may be configured to provide a personalized and optimized approach to adjusting insulin dosages for an individual based on the identified first pattern 1246 (e.g., pregnant, menstruating, ill, etc.) to achieve optimal glucose control. First titration profile 1266 may be further configured to increase or decrease an insulin dose to adjust or maintain glucose levels of the individual within an optimal target range (e.g., target glucose level within 90-110 mg/dL). In some aspects, model 1200 may generate first titration profile 1266 based on first pattern 1246 determined by cluster analysis 1240. In some aspects, for example, model 1200 may utilize one or more titration algorithms (e.g., fixed-dose titration, treat-to-target titration, CR and CF titration, etc.), using the glycemic outcomes and insulin dosing regimen of first pattern 1246 and user data 1220 as inputs, to generate first titration profile 1266.

As shown in FIG. 12C, first plot 1260 shows glucose level (mg/dL) 1262 as a function of insulin dose (U) 1264 (1 U of insulin refers to 0.01 mL of insulin). First plot 1260 includes first titration profile 1266 corresponding to a graphical representation or curve of how glucose levels of an individual are affected by a change in insulin dose (U). In some aspects, as shown in FIG. 12C, first titration profile 1266 may include equivalence point 1268 corresponding to a point on the curve for optimal dosing (e.g., glucose level of 100 mg/dL for an insulin dose of 30 U).

FIG. 12C shows first plot 1280 of second titration profile 1286, according to an example aspect. Second titration profile 1286 may be configured to provide a personalized and optimized approach to adjusting insulin dosages for an individual based on the identified second pattern 1248 (e.g., not pregnant, not menstruating, not ill, etc.) to achieve optimal glucose control. Second titration profile 1286 may be further configured to increase or decrease an insulin dose to adjust or maintain glucose levels of the individual within an optimal target range (e.g., target glucose level within 90-110 mg/dL). In some aspects, model 1200 may generate second titration profile 1286 based on second pattern 1248 determined by cluster analysis 1240. In some aspects, for example, model 1200 may utilize one or more titration algorithms (e.g., fixed-dose titration, treat-to-target titration, CR and CF titration, etc.), using the glycemic outcomes and insulin dosing regimen of second pattern 1248 and user data 1220 as inputs, to generate second titration profile 1286.

As shown in FIG. 12C, second plot 1280 shows glucose level (mg/dL) 1282 as a function of insulin dose (U) 1284 (1 U of insulin refers to 0.01 mL of insulin). Second plot 1280 includes second titration profile 1286 corresponding to a graphical representation or curve of how glucose levels of an individual are affected by a change in insulin dose (U). In some aspects, as shown in FIG. 12C, second titration profile 1286 may include equivalence point 1288 corresponding to a point on the curve for optimal dosing (e.g., glucose level of 100 mg/dL for an insulin dose of 20 U).

In some aspects, first titration profile 1266 may include the user being pregnant. In some aspects, first titration profile 1266 may include the user menstruating. In some aspects, first titration profile 1266 may include the user being ill. In some aspects, second titration profile 1286 may include the user not being pregnant. In some aspects, second titration profile 1286 may include the user not menstruating. In some aspects, second titration profile 1286 may include the user not being ill. In some aspects, first titration profile 1266 may include the user being pregnant and second titration profile 1286 may include the user not being pregnant. In some aspects, first titration profile 1266 may include the user menstruating and second titration profile 1286 may include the user not menstruating. In some aspects, first titration profile 1266 may include the user being ill and second titration profile 1286 may include the user not being ill.

In some aspects, the two or more patterns identified by cluster analysis 1240 may include the user being pregnant and the user not being pregnant, for example, first pattern 1246 may include the user being pregnant and second pattern 1248 may include the user not being pregnant. In some aspects, first titration profile 1266 may correspond to the user being pregnant and second titration profile 1286 may correspond to the user not being pregnant. In some aspects, as shown in FIG. 12C, first titration profile 1266 (e.g., user being pregnant) may have a higher insulin dose range (e.g., glucose level of 100 mg/dL for an insulin dose of 30 U at equivalence point 1268) than second titration profile 1286 (e.g., glucose level of 100 mg/dL for an insulin dose of 20 U at equivalence point 1288).

In some aspects, the two or more patterns identified by cluster analysis 1240 may include the user menstruating and the user not menstruating, for example, first pattern 1246 may include the user menstruating and second pattern 1248 may include the user not menstruating. In some aspects, first titration profile 1266 may correspond to the user menstruating and second titration profile 1286 may correspond to the user not menstruating. In some aspects, as shown in FIG. 12C, first titration profile 1266 (e.g., user menstruating) may have a higher insulin dose range (e.g., glucose level of 100 mg/dL for an insulin dose of 30 U at equivalence point 1268) than second titration profile 1286 (e.g., glucose level of 100 mg/dL for an insulin dose of 20 U at equivalence point 1288).

In some aspects, the two or more patterns identified by cluster analysis 1240 may include the user being ill and the user not being ill, for example, first pattern 1246 may include the user being ill and second pattern 1248 may include the user not being ill. In some aspects, first titration profile 1266 may correspond to the user being ill and second titration profile 1286 may correspond to the user not being ill. In some aspects, second titration profile 1086 may be shifted in time (e.g., by one or more time zones) relative to first titration profile 1066. In some aspects, as shown in FIG. 12C, first titration profile 1266 (e.g., user being ill) may have a higher insulin dose range (e.g., glucose level of 100 mg/dL for an insulin dose of 30 U at equivalence point 1268) than second titration profile 1286 (e.g., glucose level of 100 mg/dL for an insulin dose of 20 U at equivalence point 1288). In some aspects, first titration profile 1266 (e.g., user being ill) may have a lower insulin dose range (e.g., glucose level of 100 mg/dL for an insulin dose of 20 U) than second titration profile 1286 (e.g., glucose level of 100 mg/dL for an insulin dose of 30 U).

In some aspects, model 1200 may further include dynamically updating first titration profile 1266 in real time based on a variability of first titration profile 1266 over time. In some aspects, for example, the variability may include a threshold above which first titration profile 1266 is updated based on an updated first pattern 1246 that includes the recent variability. For example, the threshold may be at least 20%, at least 25%, or at least 30%. In some aspects, the threshold is in a range from about 20% to about 30%. In some aspects, the threshold is in a range from about 15% to about 40%.

In some aspects, model 1200 may further include generating a new titration profile when a variability of one of the titration profiles is above a threshold. In some aspects, the threshold is in a range from about 20% to about 30%. In some aspects, the threshold is in a range from about 15% to about 40%. In some aspects, model 1200 may automatically generate the new titration profile when the threshold is exceeded. In some aspects, model 1200 may send a communication to the user to confirm the new titration profile should be generated and/or incorporated into the therapy regimen upon user confirmation.

In some aspects, model 1200 may include a machine learning model. In some aspects, for example, analyte pattern analysis 1210, user data 1220, cluster analysis 1240, and generation of first and second titration profiles 1266, 1286 may be performed by a machine learning model. In some aspects, the machine learning model may include an unsupervised machine learning model performing cluster analysis.

In some aspects, analyte pattern analysis 1210, user data 1220, cluster analysis 1240, and generation of first and second titration profiles 1266, 1286 of model 1200 may be performed by a generative AI model. In some aspects, model 1200 may include prompting the generative AI model to determine whether the plurality of analyte profiles (e.g., glucose segment I 1216 and glucose segment J 1218) of analyte pattern analysis 1210 are correlated to user data 1220. In some aspects, for example, model 1200 may include prompting the generative AI model to determine whether the plurality of analyte profiles (e.g., glucose segment I 1216 and glucose segment J 1218) of analyte pattern analysis 1210 are correlated to the user being pregnant or the user not being pregnant. In some aspects, for example, model 1200 may include prompting the generative AI model to determine whether the plurality of analyte profiles (e.g., glucose segment I 1216 and glucose segment J 1218) of analyte pattern analysis 1210 are correlated to the user menstruating or the user not menstruating. In some aspects, for example, model 1200 may include prompting the generative AI model to determine whether the plurality of analyte profiles (e.g., glucose segment I 1216 and glucose segment J 1218) of analyte pattern analysis 1210 are correlated to the user being ill or the user not being ill.

In some aspects, model 1200 may include prompting the generative AI model to determine whether the plurality of analyte profiles (e.g., glucose segment I 1216 and glucose segment J 1218) of analyte pattern analysis 1210 are correlated to user data 1220 (e.g., pregnant, not pregnant, menstruating, not menstruating, ill, not ill, etc.). In some aspects, model 1200 may include prompting the generative AI model to determine whether the two or more patterns (e.g., first and second patterns 1246, 1248) are correlated to a second type of user data. For example, model 1200 may determine whether first pattern 1246 (e.g., pregnant) and second pattern 1248 (e.g., not pregnant) are correlated to a second type of user data (e.g., pregnant and ill, pregnant and not menstruating, etc.).

In some aspects, analyte pattern analysis 1210, user data 1220, cluster analysis 1240, and generation of first and second titration profiles 1266, 1286 of model 1200 may be performed by a LLM (e.g., GPT, Claude, Gemini, Copilot, DeepSeek, etc.) that compares the two or more identified patterns (e.g., first and second patterns 1246, 1248) to a second type of user data to determine a level of correlation. For example, model 1200 may utilize the LLM to compare first pattern 1246 (e.g., pregnant) and second pattern 1248 (e.g., not pregnant) to a second type of user data, for example, illness (e.g., gestational diabetes, high blood pressure, urinary tract infection, preeclampsia, hyperemesis gravidarum (“morning sickness”), etc.), and determine the level of correlation.

In some aspects, model 1200 may utilize the LLM to generate or update first titration profile 1266 based on the level of correlation between first pattern 1246 (e.g., pregnant) and the second type of user data (e.g., pregnant and ill, etc.). In some aspects, for example, model 1200 may utilize the LLM to implement one or more titration algorithms (e.g., fixed-dose titration, treat-to-target titration, CR and CF titration, etc.), using the glycemic outcomes and insulin dosing regimen of first pattern 1246 and user data 1220 as inputs, to generate first titration profile 1266. In some aspects, for example, model 1200 may utilize the LLM to implement one or more titration algorithms (e.g., fixed-dose titration, treat-to-target titration, CR and CF titration, etc.), using the glycemic outcomes and insulin dosing regimen of first pattern 1246 and the level of correlation of first pattern 1246 (e.g., pregnant) to the second type of user data (e.g., ill, etc.) as inputs, to generate or update first titration profile 1266.

### Example Flow Diagram for Pregnancy Titration Profiles

As discussed above, a problem in the art is that current daily monitoring schemes do not correlate day-to-day analyte pattern variations with specific user data, for example, whether the user is pregnant or not pregnant. Also, current daily monitoring schemes do not consider pregnancy data to identify distinct patterns and generate specific titration profiles based on those identified patterns (e.g., pregnant vs. not pregnant).

Pregnancy may affect a dosing regimen and a titration profile of an individual over time. For example, as the individual's weight increases during the pregnancy (e.g., after about 20 weeks pregnant), more insulin is needed since dosing is a function of total body weight (e.g., starting dose is 0.3 IU per kg body weight per day). Eventually, the individual may need 2-3 times the daily amount of insulin the individual took before they were pregnant. Further, the individual's insulin sensitivity (e.g., ISF) may decrease over time during the pregnancy. Hormones produced by the placenta may interfere with how the individual normally absorbs insulin since some of food is now being directed to the fetus, and more insulin is needed to pass into the individual's own cells.

In order to address this problem, the systems and methods of the present disclosure identify two patterns (e.g., pregnant and not pregnant) associated with an individual's multi-day analyte data (e.g., glucose data) and pregnancy data, and generate improved titration profiles for each identified pattern. This provides an improvement to diabetes management technology by correlating the analyte data to whether the individual is pregnant or not pregnant, and optimizing a titration profile for each specific pattern. For example, the system may identify a pregnant analyte pattern distinguishable from a not pregnant analyte pattern, and then generate a corresponding pregnant titration profile based on the pregnant analyte pattern and a not pregnant titration profile based on the not pregnant analyte pattern. Further, the present disclosure may dynamically update the pregnant titration profile in real time based on a variability of that titration profile over time (e.g., at the first trimester (1 to 13 weeks), at the second trimester (14 to 27 weeks), at the third trimester (28 to 40 weeks)). This provides an improvement to diabetes management technology by continually adjusting and maintaining that pregnant titration profile within an optimal target range over time throughout the pregnancy to accommodate the individual's changing therapy needs.

FIG. 13 illustrates flow diagram 1300, according to an example aspect. For example, flow diagram 1300 may be for analyte pattern analysis system 400 shown in FIG. 4. For example, flow diagram 1300 may be for software application 470 shown in FIG. 4. Flow diagram 1300 may be configured to identify analyte patterns (e.g., glucose patterns). Flow diagram 1300 may be further configured to analyze analyte profiles of analyte data of a user and group the analyte patterns into a pregnant pattern and a not pregnant pattern. Flow diagram 1300 may be further configured to develop personalized and optimized titration profiles for adaptive dose guidance for an individual. Flow diagram 1300 may be further configured to identify a pregnant pattern and a not pregnant pattern, and generate a pregnant titration profile based on the pregnant pattern and a not pregnant titration profile based on the not pregnant pattern. Flow diagram 1300 may be further configured to streamline analyte pattern analysis (e.g., glucose pattern analysis) and generation of the titration profiles using a model (e.g., model 1200, generative AI model, LLM).

It is to be appreciated that not all operations in FIG. 13 are needed to perform the disclosure provided herein. Further, some of the operations may be performed simultaneously, sequentially, and/or in a different order than shown in FIG. 13. Flow diagram 1300 shall be described with reference to FIGS. 4-11 and 12A-12C. However, flow diagram 1300 is not limited to those example aspects. Although flow diagram 1300 is shown in FIG. 13 as a stand-alone method, the aspects of this disclosure may be used with other apparatuses, systems, and/or methods, such as, but not limited to, elements in FIGS. 1-11, 12A-12C, 14, and 15, e.g., analyte monitoring system 100, sensor control device 102, receiver device 200, analyte pattern analysis system 400, flow diagram 500, machine learning model 600, cluster plot 700, calendar plot 800, distance plot 900, model 1000, flow diagram 1100, model 1200, flow diagram 1400, and/or flow diagram 1500. In some aspects, flow diagram 1300 may be implemented by analyte pattern analysis system 400 shown in FIG. 4 (e.g., via a processor or software application 470). In some aspects, flow diagram 1300 may be implemented by software application 470 shown in FIG. 4. In some aspects, flow diagram 1300 may be implemented by model 1200 shown in FIGS. 12A-12C.

In operation 1302, as shown in the example of FIGS. 4-11 and 12A-12C, analyte levels of a user may be monitored by an analyte monitoring device (e.g., OBU 410 shown in FIG. 4). For example, daily glucose profiles of the user may be monitored.

In operation 1304, as shown in the example of FIGS. 4-11 and 12A-12C, user data of the user may be received by at least one processor (e.g., receiver device 450 running software application 470 shown in FIG. 4), including analyte data monitored by the analyte monitoring device and pregnancy data (e.g., user data 1220 shown in FIG. 12A). For example, the user data may include analyte data of the user, for example, glucose data (e.g., daily glucose profiles) and pregnancy data (e.g., whether the user is pregnant or not pregnant).

In some aspects, the pregnancy data may indicate whether the user is pregnant or not pregnant. In some aspects, for example, the pregnancy data may include how long the user has been pregnant (e.g., first trimester (1 to 13 weeks), second trimester (14 to 27 weeks), third trimester (28 to 40 weeks)). In some aspects, for example, the pregnancy data may include how many fetuses are developing during the pregnancy (e.g., twin pregnancy, triplets, etc.). In some aspects, the pregnancy data may include a change (e.g. an increase) in total body weight. In some aspects, the pregnancy data may indicate whether the user has a decreased insulin sensitivity (e.g., ISF).

In some aspects, the pregnancy data (e.g., user data 1220) may be received via a user query and/or determined via one or more monitoring devices. In some aspects, for example, a query may be outputted to the user requesting pregnancy data (e.g., via receiver device 450 shown in FIG. 4). In some aspects, the pregnancy data may be received from the user based on one or more responses to the query. In some aspects, for example, the query may include one or more questions regarding pregnancy (e.g., “Are you pregnant or not pregnant?”, “How far along is the pregnancy?”, “What is your current total body weight?”, “How many fetuses are developing?”, “Is your insulin resistance increasing?”, “What is your current ISF?”, etc.). In some aspects, the pregnancy data may be determined via one or more monitoring devices (e.g., first smart device 430, second smart device 440, and/or receiver device 450 shown in FIG. 4). In some aspects, the pregnancy data may be received from a HCP.

In some aspects, after receiving user confirmation that the user is pregnant, the system will assume that all glucose profiles for a period of time following the user confirmation are glucose profiles associated with pregnancy. In some aspects, for example, the system may prompt the user to confirm they are still pregnant, for example, after every 30 days or at critical phases of the pregnancy (e.g., at first trimester, at second trimester, at third trimester, etc.). In some aspects, after 8-9 months, the system may query the user regarding the pregnancy and whether the user is resuming a non-pregnant pattern. In some aspects, the system may track the timing of the user's pregnancy and follow-up with the user around 8-9 months. In some aspects, the system may consult EMR data of the user to determine the status of the pregnancy and whether therapy based on the pregnancy pattern (e.g., pregnancy titration profile) should continue or if the therapy should switch to a non-pregnant pattern.

In operation 1306, as shown in the example of FIGS. 4-11 and 12A-12C, analyte profiles (e.g., glucose segment I 1216 and glucose segment J 1218 shown in FIG. 12A) of the analyte data and the pregnancy data (e.g., user data 1220 shown in FIG. 12A) over an analysis period (e.g., 14-30 days) may be analyzed by a model (e.g., model 1200 shown in FIGS. 12A-12C). For example, the analyte profiles may include daily glucose profiles.

In some aspects, the model may receive the analyte profiles (e.g., daily glucose profiles) and the pregnancy data, and correlate the analyte profiles to the pregnancy data and group them into two patterns (e.g., a pregnant pattern and a not pregnant pattern). The grouping may be performed based on one or more metrics for each analyte profile (e.g., daily glucose profile) and the pregnancy data. In some aspects, the one or more metrics may include a similar mean glucose. In some aspects, the one or more metrics may include a similar glucose variability. In some aspects, the one or more metrics may include a minimum peak glucose level. In some aspects, the model may include assessing a distance between the daily glucose profiles. For example, the distance may be based on a MARD (e.g., Equation 3 above). In some aspects, first and second daily glucose profiles (e.g., glucose segment J 1218 shown in FIG. 12A) and the pregnancy data (e.g., user data 1220 shown in FIG. 12A) may be grouped into a pregnant pattern type (e.g., first pattern 1246) when the distance is at or below a threshold. For example, the threshold may be based on a predetermined analyte value (e.g., glucose level of 30 mg/dL, glucose difference of 30 mg/dL). In some aspects, the model may include unsupervised machine learning (e.g., no training). In some aspects, the model may include supervised machine learning (e.g., with training). In some aspects, the model may include a generative AI model (e.g., LLM).

In operation 1308, as shown in the example of FIGS. 4-11 and 12A-12C, the daily analyte profiles may be grouped into a pregnant pattern (e.g., first pattern 1246 shown in FIG. 12B) and a not pregnant pattern (e.g., second pattern 1248 shown in FIG. 12B) based on the analysis performed by the model (e.g., model 1200 shown in FIGS. 12A-12C). In some aspects, the grouping may be further limited based on the pregnancy data (e.g., user data 1220 shown in FIG. 12A), for example, the daily analyte profiles may be labeled as pregnant or not pregnant and grouped accordingly.

In operation 1310, as shown in the example of FIGS. 4-11 and 12A-12C, titration profiles (e.g., first and second titration profiles 1266, 1286 shown in FIG. 12C) each corresponding to one of the two identified patterns (e.g., first and second patterns 1246, 1248 shown in FIG. 12B) may be generated by the model (e.g., model 1200 shown in FIGS. 12A-12C).

In some aspects, the model may generate first titration profile 1266 (e.g., pregnant) based on first pattern 1246 determined by cluster analysis 1240. In some aspects, for example, model 1200 may utilize one or more titration algorithms (e.g., fixed-dose titration, treat-to-target titration, CR and CF titration, etc.), using the glycemic outcomes and insulin dosing regimen of first pattern 1246 as inputs, to generate first titration profile 1266. In some aspects, the model may generate second titration profile 1286 (e.g., not pregnant) based on second pattern 1248 determined by cluster analysis 1240. In some aspects, for example, model 1200 may utilize one or more titration algorithms (e.g., fixed-dose titration, treat-to-target titration, CR and CF titration, etc.), using the glycemic outcomes and insulin dosing regimen of second pattern 1248 as inputs, to generate second titration profile 1286.

In some aspects, for example, the model may utilize a LLM to implement one or more titration algorithms (e.g., fixed-dose titration, treat-to-target titration, CR and CF titration, etc.), using the glycemic outcomes and insulin dosing regimen of first pattern 1246 and/or second pattern 1248 as inputs, to generate first titration profile 1266 and/or second titration profile 1286. In some aspects, for example, the model may utilize the LLM to implement one or more titration algorithms (e.g., fixed-dose titration, treat-to-target titration, CR and CF titration, etc.), using the glycemic outcomes and insulin dosing regimen of first pattern 1246 and a level of correlation of first pattern 1246 (e.g., pregnant) to a second type of user data (e.g., ill, etc.) as inputs, to generate or update first titration profile 1266.

In operation 1312, optionally, as shown in the example of FIGS. 4-11 and 12A-12C, one or more of the titration profiles (e.g., first and second titration profiles 1266, 1286 shown in FIG. 12C) may be dynamically updated in real time by the model based on a variability of a specific titration profile over time.

In some aspects, the model may include dynamically updating first titration profile 1266 (e.g., pregnant) in real time based on a variability of first titration profile 1266 over time. In some aspects, for example, the variability may include a threshold above which first titration profile 1266 is updated based on an updated first pattern 1246 that includes the recent variability. For example, the threshold may be at least 20%, at least 25%, or at least 30%. In some aspects, the threshold is in a range from about 20% to about 30%. In some aspects, the threshold is in a range from about 15% to about 40%.

In some aspects, the model may further include generating a new titration profile when a variability of one of the titration profiles is above a threshold. In some aspects, the threshold is in a range from about 20% to about 30%. In some aspects, the threshold is in a range from about 15% to about 40%. In some aspects, the model may automatically generate the new titration profile when the threshold is exceeded. In some aspects, the model may send a communication to the user to confirm the new titration profile should be generated and/or incorporated into the therapy regimen upon user confirmation.

In some aspects, the system may further include outputting data to the user based on the titration profile. In some aspects, for example, the system may output a dose recommendation (e.g., on a display device) to the user based on the titration profile. In some aspects, for example, the dose recommendation may be automatically provided to the user when the titration profile is updated. In some aspects, for example, the user may request a dose recommendation and the system may consult the titration profile and provide a dose recommendation (e.g., fixed insulin dose amount). In some aspects, for example, the system may output a dose recommendation for each meal (e.g., breakfast, lunch, dinner, weekday breakfast, weekday lunch, weekday dinner, weekend breakfast, weekend lunch, weekend dinner) based on the updated titration profile. In some aspects, the system may output a daily dosing regimen (e.g., breakfast 10 U, lunch 12 U, dinner 15 U, weekday breakfast 10 U, weekday lunch 12 U, weekday dinner 15 U, weekend breakfast 10 U, weekend lunch 12 U, weekend dinner 15 U, etc.), on a display device, based on the titration profile. In some aspects, the system may output a long acting (e.g., basal) insulin dose recommendation based on the titration profile. In some aspects, the system may output a rapid-acting insulin dose (e.g., fast-acting bolus) recommendation based on the titration profile. In some aspects, for example, the system may output the titration profile on a display.

In some aspects, the system may adjust one or more insulin therapy settings (e.g., dose amount, ISF, CR) of a medication delivery device based on the titration profile. In some aspects, for example, the system may adjust one or more insulin therapy settings (e.g., dose amount, ISF, CR) to optimize a time in range (TIR) of the user based on the titration profile. In some aspects, for example, the system may output, on a display, the adjusted one or more insulin therapy settings.

### Example Flow Diagram for Menstruation Titration Profiles

As discussed above, a problem in the art is that current daily monitoring schemes do not correlate day-to-day analyte pattern variations with specific user data, for example, whether the user is menstruating or not menstruating. Also, current daily monitoring schemes do not consider menstruation data to identify distinct patterns and generate specific titration profiles based on those identified patterns (e.g., menstruating vs. not menstruating).

Menstruation may affect a dosing regimen and a titration profile of an individual over time. For example, hormonal fluctuations during a menstrual cycle may affect insulin sensitivity, and insulin doses may need to be adjusted accordingly. Generally, during the luteal phase (latter half of the cycle), insulin sensitivity decreases which can lead to higher glucose levels and a need for more insulin. Further, women with type 1 diabetes may experience increased insulin resistance and risk of hyperglycemia, and larger insulin sensitivity fluctuations over time. Additionally, each phase of the menstrual cycle (menstrual phase, follicular phase, ovulation phase, luteal phase) are characterized by distinct hormonal changes that may impact overall well-being and insulin therapy.

In order to address this problem, the systems and methods of the present disclosure identify two patterns (e.g., menstruating and not menstruating) associated with an individual's multi-day analyte data (e.g., glucose data) and menstruation data, and generate improved titration profiles for each identified pattern. This provides an improvement to diabetes management technology by correlating the analyte data to whether the individual is menstruating or not menstruating, and optimizing a titration profile for each specific pattern. For example, the system may identify a menstruating analyte pattern distinguishable from a not menstruating analyte pattern, and then generate a corresponding menstruating titration profile based on the menstruating analyte pattern and a not menstruating titration profile based on the not menstruating analyte pattern. Further, the present disclosure may dynamically update the menstruating titration profile in real time based on a variability of that titration profile over time (e.g., at the menstrual phase, at the follicular phase, at the ovulation phase, at the luteal phase). This provides an improvement to diabetes management technology by continually adjusting and maintaining that menstruating titration profile within an optimal target range over time throughout the menstrual cycle to accommodate the individual's changing therapy needs.

FIG. 14 illustrates flow diagram 1400, according to an example aspect. For example, flow diagram 1400 may be for analyte pattern analysis system 400 shown in FIG. 4. For example, flow diagram 1400 may be for software application 470 shown in FIG. 4. Flow diagram 1400 may be configured to identify analyte patterns (e.g., glucose patterns). Flow diagram 1400 may be further configured to analyze analyte profiles of analyte data of a user and group the analyte patterns into a menstruating pattern and a not menstruating pattern. Flow diagram 1400 may be further configured to develop personalized and optimized titration profiles for adaptive dose guidance for an individual. Flow diagram 1400 may be further configured to identify a menstruating pattern and a not menstruating pattern, and generate a menstruating titration profile based on the menstruating pattern and a not menstruating titration profile based on the not menstruating pattern. Flow diagram 1400 may be further configured to streamline analyte pattern analysis (e.g., glucose pattern analysis) and generation of the titration profiles using a model (e.g., model 1200, generative AI model, LLM).

It is to be appreciated that not all operations in FIG. 14 are needed to perform the disclosure provided herein. Further, some of the operations may be performed simultaneously, sequentially, and/or in a different order than shown in FIG. 14. Flow diagram 1400 shall be described with reference to FIGS. 4-11 and 12A-12C. However, flow diagram 1400 is not limited to those example aspects. Although flow diagram 1400 is shown in FIG. 14 as a stand-alone method, the aspects of this disclosure may be used with other apparatuses, systems, and/or methods, such as, but not limited to, elements in FIGS. 1-11, 12A-12C, 13, and 15, e.g., analyte monitoring system 100, sensor control device 102, receiver device 200, analyte pattern analysis system 400, flow diagram 500, machine learning model 600, cluster plot 700, calendar plot 800, distance plot 900, model 1000, flow diagram 1100, model 1200, flow diagram 1300, and/or flow diagram 1500. In some aspects, flow diagram 1400 may be implemented by analyte pattern analysis system 400 shown in FIG. 4 (e.g., via a processor or software application 470). In some aspects, flow diagram 1400 may be implemented by software application 470 shown in FIG. 4. In some aspects, flow diagram 1400 may be implemented by model 1200 shown in FIGS. 12A-12C.

In operation 1402, as shown in the example of FIGS. 4-11 and 12A-12C, analyte levels of a user may be monitored by an analyte monitoring device (e.g., OBU 410 shown in FIG. 4). For example, daily glucose profiles of the user may be monitored.

In operation 1404, as shown in the example of FIGS. 4-11 and 12A-12C, user data of the user may be received by at least one processor (e.g., receiver device 450 running software application 470 shown in FIG. 4), including analyte data monitored by the analyte monitoring device and menstruation data (e.g., user data 1220 shown in FIG. 12A). For example, the user data may include analyte data of the user, for example, glucose data (e.g., daily glucose profiles) and menstruation data (e.g., whether the user is menstruating or not menstruating).

In some aspects, the menstruation data may indicate whether the user is menstruating or not menstruating. In some aspects, for example, the menstruation data may include what phase of the menstrual cycle the user is experiencing (e.g., menstrual phase, follicular phase, ovulation phase, luteal phase). In some aspects, the menstruation data may indicate whether the user is experiencing increased insulin resistance. In some aspects, for example, the menstruation data may indicate whether the user has a decreased insulin sensitivity (e.g., ISF), for example, during the luteal phase.

In some aspects, the menstruation data (e.g., user data 1220) may be received via a user query and/or determined via one or more monitoring devices. In some aspects, for example, a query may be outputted to the user requesting menstruation data (e.g., via receiver device 450 shown in FIG. 4). In some aspects, the menstruation data may be received from the user based on one or more responses to the query. In some aspects, for example, the query may include one or more questions regarding menstruation (e.g., “Are you menstruating or not menstruating?”, “What phase of the menstrual cycle are you experiencing?”, “Have you been diagnosed with type 1 diabetes?”, “Is your insulin resistance increasing?”, “What is your current ISF?”, etc.). In some aspects, the menstruation data may be determined via one or more monitoring devices (e.g., first smart device 430, second smart device 440, and/or receiver device 450 shown in FIG. 4). In some aspects, the menstruation data may be received from a HCP or the user's EMR.

In operation 1406, as shown in the example of FIGS. 4-11 and 12A-12C, analyte profiles (e.g., glucose segment I 1216 and glucose segment J 1218 shown in FIG. 12A) of the analyte data and the menstruation data (e.g., user data 1220 shown in FIG. 12A) over an analysis period (e.g., 14-30 days) may be analyzed by a model (e.g., model 1200 shown in FIGS. 12A-12C). For example, the analyte profiles may include daily glucose profiles.

In some aspects, the model may receive the analyte profiles (e.g., daily glucose profiles) and the menstruation data, and correlate the analyte profiles to the menstruation data and group them into two patterns (e.g., a menstruating pattern and a not menstruating pattern). The grouping may be performed based on one or more metrics for each analyte profile (e.g., daily glucose profile) and the menstruation data. In some aspects, the one or more metrics may include a similar mean glucose. In some aspects, the one or more metrics may include a similar glucose variability. In some aspects, the one or more metrics may include a minimum peak glucose level. In some aspects, the model may include assessing a distance between the daily glucose profiles. For example, the distance may be based on a MARD (e.g., Equation 3 above). In some aspects, first and second daily glucose profiles (e.g., glucose segment J 1218 shown in FIG. 12A) and the menstruation data (e.g., user data 1220 shown in FIG. 12A) may be grouped into a menstruating pattern type (e.g., first pattern 1246) when the distance is at or below a threshold. For example, the threshold may be based on a predetermined analyte value (e.g., glucose level of 30 mg/dL, glucose difference of 30 mg/dL). In some aspects, the model may include unsupervised machine learning (e.g., no training). In some aspects, the model may include supervised machine learning (e.g., with training). In some aspects, the model may include a generative AI model (e.g., LLM).

In operation 1408, as shown in the example of FIGS. 4-11 and 12A-12C, the daily analyte profiles may be grouped into a menstruating pattern (e.g., first pattern 1246 shown in FIG. 12B) and a not menstruating pattern (e.g., second pattern 1248 shown in FIG. 12B) based on the analysis performed by the model (e.g., model 1200 shown in FIGS. 12A-12C). In some aspects, the grouping may be further limited based on the menstruation data (e.g., user data 1220 shown in FIG. 12A), for example, the daily analyte profiles may be labeled as menstruating or not menstruating and grouped accordingly. In some aspects, a glucose profile that the user confirms as corresponding to menstruation may be used by the system to determine that another similar glucose profile also corresponds to menstruation and both glucose profiles may be grouped into the same menstruating pattern, for example, without having the user confirm every day.

In operation 1410, as shown in the example of FIGS. 4-11 and 12A-12C, titration profiles (e.g., first and second titration profiles 1266, 1286 shown in FIG. 12C) each corresponding to one of the two identified patterns (e.g., first and second patterns 1246, 1248 shown in FIG. 12B) may be generated by the model (e.g., model 1200 shown in FIGS. 12A-12C).

In some aspects, the model may generate first titration profile 1266 (e.g., menstruating) based on first pattern 1246 determined by cluster analysis 1240. In some aspects, for example, model 1200 may utilize one or more titration algorithms (e.g., fixed-dose titration, treat-to-target titration, CR and CF titration, etc.), using the glycemic outcomes and insulin dosing regimen of first pattern 1246 as inputs, to generate first titration profile 1266. In some aspects, the model may generate second titration profile 1286 (e.g., not menstruating) based on second pattern 1248 determined by cluster analysis 1240. In some aspects, for example, model 1200 may utilize one or more titration algorithms (e.g., fixed-dose titration, treat-to-target titration, CR and CF titration, etc.), using the glycemic outcomes and insulin dosing regimen of second pattern 1248 as inputs, to generate second titration profile 1286.

In some aspects, for example, the model may utilize a LLM to implement one or more titration algorithms (e.g., fixed-dose titration, treat-to-target titration, CR and CF titration, etc.), using the glycemic outcomes and insulin dosing regimen of first pattern 1246 and/or second pattern 1248 as inputs, to generate first titration profile 1266 and/or second titration profile 1286. In some aspects, for example, the model may utilize the LLM to implement one or more titration algorithms (e.g., fixed-dose titration, treat-to-target titration, CR and CF titration, etc.), using the glycemic outcomes and insulin dosing regimen of first pattern 1246 and a level of correlation of first pattern 1246 (e.g., menstruating) to a second type of user data (e.g., ill, etc.) as inputs, to generate or update first titration profile 1266.

In operation 1412, optionally, as shown in the example of FIGS. 4-11 and 12A-12C, one or more of the titration profiles (e.g., first and second titration profiles 1266, 1286 shown in FIG. 12C) may be dynamically updated in real time by the model based on a variability of a specific titration profile over time.

In some aspects, the model may include dynamically updating first titration profile 1266 (e.g., menstruating) in real time based on a variability of first titration profile 1266 over time. In some aspects, for example, the variability may include a threshold above which first titration profile 1266 is updated based on an updated first pattern 1246 that includes the recent variability. For example, the threshold may be at least 20%, at least 25%, or at least 30%. In some aspects, the threshold is in a range from about 20% to about 30%. In some aspects, the threshold is in a range from about 15% to about 40%.

In some aspects, the model may further include generating a new titration profile when a variability of one of the titration profiles is above a threshold. In some aspects, the threshold is in a range from about 20% to about 30%. In some aspects, the threshold is in a range from about 15% to about 40%. In some aspects, the model may automatically generate the new titration profile when the threshold is exceeded. In some aspects, the model may send a communication to the user to confirm the new titration profile should be generated and/or incorporated into the therapy regimen upon user confirmation.

In some aspects, the system may further include outputting data to the user based on the titration profile. In some aspects, for example, the system may output a dose recommendation (e.g., on a display device) to the user based on the titration profile. In some aspects, for example, the dose recommendation may be automatically provided to the user when the titration profile is updated. In some aspects, for example, the user may request a dose recommendation and the system may consult the titration profile and provide a dose recommendation (e.g., fixed insulin dose amount). In some aspects, for example, the system may output a dose recommendation for each meal (e.g., breakfast, lunch, dinner, weekday breakfast, weekday lunch, weekday dinner, weekend breakfast, weekend lunch, weekend dinner) based on the updated titration profile. In some aspects, the system may output a daily dosing regimen (e.g., breakfast 10 U, lunch 12 U, dinner 15 U, weekday breakfast 10 U, weekday lunch 12 U, weekday dinner 15 U, weekend breakfast 10 U, weekend lunch 12 U, weekend dinner 15 U, etc.), on a display device, based on the titration profile. In some aspects, the system may output a long acting (e.g., basal) insulin dose recommendation based on the titration profile. In some aspects, the system may output a rapid-acting insulin dose (e.g., fast-acting bolus) recommendation based on the titration profile. In some aspects, for example, the system may output the titration profile on a display.

In some aspects, the system may adjust one or more insulin therapy settings (e.g., dose amount, ISF, CR) of a medication delivery device based on the titration profile. In some aspects, for example, the system may adjust one or more insulin therapy settings (e.g., dose amount, ISF, CR) to optimize a time in range (TIR) of the user based on the titration profile. In some aspects, for example, the system may output, on a display, the adjusted one or more insulin therapy settings.

### Example Flow Diagram for Illness Titration Profiles

As discussed above, a problem in the art is that current daily monitoring schemes do not correlate day-to-day analyte pattern variations with specific user data, for example, whether the user is ill or not ill. Also, current daily monitoring schemes do not consider illness data to identify distinct patterns and generate specific titration profiles based on those identified patterns (e.g., ill vs. not ill).

Illness may affect a dosing regimen and a titration profile of an individual over time. For example, illnesses that make it difficult to retain food (e.g., COVID, SARS, influenza, rotavirus, gastroenteritis, mononucleosis, meningitis, ulcers, nausea, etc.) may require lower insulin doses, whereas illnesses that involve infections or inflammations (e.g., pneumonia, urinary tract infection, thyroid, acromegaly, obesity, polycystic ovary, etc.) may increase insulin resistance and/or affect insulin production and require higher insulin doses. Multiple illnesses may compound the issue leading to fluctuating glucose levels and changing insulin resistance.

In order to address this problem, the systems and methods of the present disclosure identify two patterns (e.g., ill and not ill) associated with an individual's multi-day analyte data (e.g., glucose data) and illness data, and generate improved titration profiles for each identified pattern. This provides an improvement to diabetes management technology by correlating the analyte data to whether the individual is ill or not ill, and optimizing a titration profile for each specific pattern. For example, the system may identify an ill analyte pattern distinguishable from a not ill analyte pattern, and then generate a corresponding ill titration profile, considering the type of illness and whether there are multiple illnesses, based on the ill analyte pattern and a not ill titration profile based on the not ill analyte pattern. Further, the present disclosure may dynamically update the ill titration profile in real time based on a variability of that titration profile over time. This provides an improvement to diabetes management technology by continually adjusting and maintaining that ill titration profile within an optimal target range over time throughout the illness to accommodate the individual's changing therapy needs.

FIG. 15 illustrates flow diagram 1500, according to an example aspect. For example, flow diagram 1500 may be for analyte pattern analysis system 400 shown in FIG. 4. For example, flow diagram 1500 may be for software application 470 shown in FIG. 4. Flow diagram 1500 may be configured to identify analyte patterns (e.g., glucose patterns). Flow diagram 1500 may be further configured to analyze analyte profiles of analyte data of a user and group the analyte patterns into an ill pattern and a not ill pattern. Flow diagram 1500 may be further configured to develop personalized and optimized titration profiles for adaptive dose guidance for an individual. Flow diagram 1500 may be further configured to identify an ill pattern and a not ill pattern, and generate an ill titration profile based on the ill pattern and a not ill titration profile based on the not ill pattern. Flow diagram 1500 may be further configured to streamline analyte pattern analysis (e.g., glucose pattern analysis) and generation of the titration profiles using a model (e.g., model 1200, generative AI model, LLM).

It is to be appreciated that not all operations in FIG. 15 are needed to perform the disclosure provided herein. Further, some of the operations may be performed simultaneously, sequentially, and/or in a different order than shown in FIG. 15. Flow diagram 1500 shall be described with reference to FIGS. 4-11 and 12A-12C. However, flow diagram 1500 is not limited to those example aspects. Although flow diagram 1500 is shown in FIG. 15 as a stand-alone method, the aspects of this disclosure may be used with other apparatuses, systems, and/or methods, such as, but not limited to, elements in FIGS. 1-11, 12A-12C, 13, and 14, e.g., analyte monitoring system 100, sensor control device 102, receiver device 200, analyte pattern analysis system 400, flow diagram 500, machine learning model 600, cluster plot 700, calendar plot 800, distance plot 900, model 1000, flow diagram 1100, model 1200, flow diagram 1300, and/or flow diagram 1400. In some aspects, flow diagram 1500 may be implemented by analyte pattern analysis system 400 shown in FIG. 4 (e.g., via a processor or software application 470). In some aspects, flow diagram 1500 may be implemented by software application 470 shown in FIG. 4. In some aspects, flow diagram 1500 may be implemented by model 1200 shown in FIGS. 12A-12C.

In operation 1502, as shown in the example of FIGS. 4-11 and 12A-12C, analyte levels of a user may be monitored by an analyte monitoring device (e.g., OBU 410 shown in FIG. 4). For example, daily glucose profiles of the user may be monitored.

In operation 1504, as shown in the example of FIGS. 4-11 and 12A-12C, user data of the user may be received by at least one processor (e.g., receiver device 450 running software application 470 shown in FIG. 4), including analyte data monitored by the analyte monitoring device and illness data (e.g., user data 1220 shown in FIG. 12A). For example, the user data may include analyte data of the user, for example, glucose data (e.g., daily glucose profiles) and illness data (e.g., whether the user is ill or not ill).

In some aspects, the illness data may indicate whether the user is ill or not ill. In some aspects, for example, the illness data may indicate the type of illness. In some aspects, for example, the illness data may include whether there are multiple illnesses and what those illnesses are. In some aspects, the illness data may indicate whether the user is receiving any medication and/or treatment for the illness (e.g., GLP-1, antibiotics, anti-inflammatory, radiotherapy, chemotherapy, etc.). In some aspects, the illness data may indicate that it is difficult for the user to retain food (e.g., vomiting, abdominal pain, etc.). In some aspects, the illness data may indicate whether the user is experiencing increased insulin resistance. In some aspects, the illness data may indicate whether the user is experiencing decreased insulin production.

In some aspects, the illness data (e.g., user data 1220) may be received via a user query and/or determined via one or more monitoring devices. In some aspects, for example, a query may be outputted to the user requesting illness data (e.g., via receiver device 450 shown in FIG. 4). In some aspects, the illness data may be received from the user based on one or more responses to the query. In some aspects, for example, the query may include one or more questions regarding illness (e.g., “Are you ill or not ill?”, “How long have you been ill?”, “What type of illness?”, “Do you have multiple illnesses?”, “Are you receiving any medication or treatment for the illness?”, “Is it difficult to retain food?”, “Is your insulin resistance increasing?”, “Is your insulin production decreasing?”, “What is your current ISF?”, etc.). In some aspects, the illness data may be determined via one or more monitoring devices (e.g., first smart device 430, second smart device 440, and/or receiver device 450 shown in FIG. 4). In some aspects, the illness data may be received from a HCP or the user's EMR.

In operation 1506, as shown in the example of FIGS. 4-11 and 12A-12C, analyte profiles (e.g., glucose segment I 1216 and glucose segment J 1218 shown in FIG. 12A) of the analyte data and the illness data (e.g., user data 1220 shown in FIG. 12A) over an analysis period (e.g., 3-30 days) may be analyzed by a model (e.g., model 1200 shown in FIGS. 12A-12C). For example, the analyte profiles may include daily glucose profiles. In some aspects, for example, the analysis period may be over a shorter time period (e.g., 3-7 days) based on the type of illness (e.g., cold, flu, fever, food poisoning, etc.). In some aspects, for example, the analysis period may be over a longer time period (e.g., 7-30 days) based on the type of illness (e.g., infection, viral, COVID, mononucleosis, Lyme disease, cancer, etc.).

In some aspects, the model may receive the analyte profiles (e.g., daily glucose profiles) and the illness data, and correlate the analyte profiles to the illness data and group them into two patterns (e.g., an ill pattern and a not ill pattern). The grouping may be performed based on one or more metrics for each analyte profile (e.g., daily glucose profile) and the illness data. In some aspects, the one or more metrics may include a similar mean glucose. In some aspects, the one or more metrics may include a similar glucose variability. In some aspects, the one or more metrics may include a minimum peak glucose level. In some aspects, the model may include assessing a distance between the daily glucose profiles. For example, the distance may be based on a MARD (e.g., Equation 3 above). In some aspects, first and second daily glucose profiles (e.g., glucose segment J 1218 shown in FIG. 12A) and the illness data (e.g., user data 1220 shown in FIG. 12A) may be grouped into an ill pattern type (e.g., first pattern 1246) when the distance is at or below a threshold. For example, the threshold may be based on a predetermined analyte value (e.g., glucose level of 30 mg/dL, glucose difference of 30 mg/dL). In some aspects, the model may include unsupervised machine learning (e.g., no training). In some aspects, the model may include supervised machine learning (e.g., with training). In some aspects, the model may include a generative AI model (e.g., LLM).

In operation 1508, as shown in the example of FIGS. 4-11 and 12A-12C, the daily analyte profiles may be grouped into an ill pattern (e.g., first pattern 1246 shown in FIG. 12B) and a not ill pattern (e.g., second pattern 1248 shown in FIG. 12B) based on the analysis performed by the model (e.g., model 1200 shown in FIGS. 12A-12C). In some aspects, the grouping may be further limited based on the illness data (e.g., user data 1220 shown in FIG. 12A), for example, the daily analyte profiles may be labeled as ill or not ill and grouped accordingly.

In operation 1510, as shown in the example of FIGS. 4-11 and 12A-12C, titration profiles (e.g., first and second titration profiles 1266, 1286 shown in FIG. 12C) each corresponding to one of the two identified patterns (e.g., first and second patterns 1246, 1248 shown in FIG. 12B) may be generated by the model (e.g., model 1200 shown in FIGS. 12A-12C).

In some aspects, the model may generate first titration profile 1266 (e.g., ill) based on first pattern 1246 determined by cluster analysis 1240. In some aspects, for example, model 1200 may utilize one or more titration algorithms (e.g., fixed-dose titration, treat-to-target titration, CR and CF titration, etc.), using the glycemic outcomes and insulin dosing regimen of first pattern 1246 as inputs, to generate first titration profile 1266. In some aspects, the model may generate second titration profile 1286 (e.g., not ill) based on second pattern 1248 determined by cluster analysis 1240. In some aspects, for example, model 1200 may utilize one or more titration algorithms (e.g., fixed-dose titration, treat-to-target titration, CR and CF titration, etc.), using the glycemic outcomes and insulin dosing regimen of second pattern 1248 as inputs, to generate second titration profile 1286.

In some aspects, for example, the model may utilize a LLM to implement one or more titration algorithms (e.g., fixed-dose titration, treat-to-target titration, CR and CF titration, etc.), using the glycemic outcomes and insulin dosing regimen of first pattern 1246 and/or second pattern 1248 as inputs, to generate first titration profile 1266 and/or second titration profile 1286. In some aspects, for example, the model may utilize the LLM to implement one or more titration algorithms (e.g., fixed-dose titration, treat-to-target titration, CR and CF titration, etc.), using the glycemic outcomes and insulin dosing regimen of first pattern 1246 and a level of correlation of first pattern 1246 (e.g., ill) to a second type of user data (e.g., pregnant, not pregnant, menstruating, not menstruating, etc.) as inputs, to generate or update first titration profile 1266.

In operation 1512, optionally, as shown in the example of FIGS. 4-11 and 12A-12C, one or more of the titration profiles (e.g., first and second titration profiles 1266, 1286 shown in FIG. 12C) may be dynamically updated in real time by the model based on a variability of a specific titration profile over time.

In some aspects, the model may include dynamically updating first titration profile 1266 (e.g., user being ill) in real time based on a variability of first titration profile 1266 over time. In some aspects, for example, the variability may include a threshold above which first titration profile 1266 is updated based on an updated first pattern 1246 that includes the recent variability. For example, the threshold may be at least 20%, at least 25%, or at least 30%. In some aspects, the threshold is in a range from about 20% to about 30%. In some aspects, the threshold is in a range from about 15% to about 40%.

In some aspects, the model may further include generating a new titration profile when a variability of one of the titration profiles is above a threshold. In some aspects, the threshold is in a range from about 20% to about 30%. In some aspects, the threshold is in a range from about 15% to about 40%. In some aspects, the model may automatically generate the new titration profile when the threshold is exceeded. In some aspects, the model may send a communication to the user to confirm the new titration profile should be generated and/or incorporated into the therapy regimen upon user confirmation.

In some aspects, the system may further include outputting data to the user based on the titration profile. In some aspects, for example, the system may output a dose recommendation (e.g., on a display device) to the user based on the titration profile. In some aspects, for example, the dose recommendation may be automatically provided to the user when the titration profile is updated. In some aspects, for example, the user may request a dose recommendation and the system may consult the titration profile and provide a dose recommendation (e.g., fixed insulin dose amount). In some aspects, for example, the system may output a dose recommendation for each meal (e.g., breakfast, lunch, dinner, weekday breakfast, weekday lunch, weekday dinner, weekend breakfast, weekend lunch, weekend dinner) based on the updated titration profile. In some aspects, the system may output a daily dosing regimen (e.g., breakfast 10 U, lunch 12 U, dinner 15 U, weekday breakfast 10 U, weekday lunch 12 U, weekday dinner 15 U, weekend breakfast 10 U, weekend lunch 12 U, weekend dinner 15 U, etc.), on a display device, based on the titration profile. In some aspects, the system may output a long acting (e.g., basal) insulin dose recommendation based on the titration profile. In some aspects, the system may output a rapid-acting insulin dose (e.g., fast-acting bolus) recommendation based on the titration profile. In some aspects, for example, the system may output the titration profile on a display.

In some aspects, the system may adjust one or more insulin therapy settings (e.g., dose amount, ISF, CR) of a medication delivery device based on the titration profile. In some aspects, for example, the system may adjust one or more insulin therapy settings (e.g., dose amount, ISF, CR) to optimize a time in range (TIR) of the user based on the titration profile. In some aspects, for example, the system may output, on a display, the adjusted one or more insulin therapy settings.

In some aspects, the processing of the systems and methods described above (e.g., machine learning model 600, model 1000, model 1200) may occur on a OBU (e.g., OBU 410), a medical delivery device (e.g., medical delivery device 420), a phone (e.g., receiver device 450), a cloud server (e.g., remote server 480), or a combination thereof.

It is to be understood that the phraseology or terminology herein is for the purpose of description and not of limitation, such that the terminology or phraseology of the present specification is to be interpreted by those skilled in relevant art(s) in light of the teachings herein.

The examples herein are illustrative, but not limiting, of the aspects of this disclosure. Other suitable modifications and adaptations of the variety of conditions and parameters normally encountered in the field, and which would be apparent to those skilled in the relevant art(s), are within the spirit and scope of the disclosure.

While specific aspects have been described above, it will be appreciated that the aspects may be practiced otherwise than as described. The description is not intended to limit the scope of the claims.

The aspects have been described above with the aid of functional building blocks illustrating the implementation of specified functions and relationships thereof. The boundaries of these functional building blocks have been arbitrarily defined herein for the convenience of the description. Alternate boundaries may be defined so long as the specified functions and relationships thereof are appropriately performed.

The foregoing description of the specific aspects will so fully reveal the general nature of the aspects that others may, by applying knowledge within the skill of the art, readily modify and/or adapt for various applications such specific aspects, without undue experimentation, without departing from the general concept of the aspects. Therefore, such adaptations and modifications are intended to be within the meaning and range of equivalents of the disclosed aspects, based on the teaching and guidance presented herein.

The breadth and scope of the aspects should not be limited by any of the above-described example aspects, but should be defined only in accordance with the following claims and their equivalents.

The present invention may also be described in accordance with the following clauses:
Clause 1. A method of identifying and reporting patterns of glucose data, the method comprising:
monitoring, by a glucose monitoring device, glucose levels of a user, wherein the glucose monitoring device comprises a first portion arranged above a skin surface of the user, and a second portion arranged below the skin surface and in contact with interstitial fluid of the user;
receiving, by at least one processor, therapy data of the user, wherein the therapy data comprises glucose data monitored by the glucose monitoring device;
analyzing, by the at least one processor, a plurality of daily glucose profiles of the glucose data over an analysis period, optionally by a machine learning model;
grouping, by the at least one processor, the daily glucose profiles into two or more patterns based on the analysis, optionally performed by the machine learning model; and
outputting, on a display device in communication with the at least one processor, a report comprising identification of one of the two or more patterns associated with each daily glucose profile.
Clause 2. The method of clause 1, wherein the report comprises a display of each daily glucose profile.
Clause 3. The method of clause 2, wherein the display overlays the two or more patterns over the analysis period and visually distinguishes each pattern from another.
Clause 4. The method of clause 3, wherein the display visually distinguishes each of the two or more patterns from another by color.
Clause 5. The method of any one of clauses 1 to 4, wherein the report comprises a calendar with an indication of one of the two or more patterns associated with the daily glucose profile for each day on the calendar.
Clause 6. The method of clause 5, wherein the calendar separates the two or more patterns into corresponding calendar days and visually distinguishes each pattern from another.
Clause 7. The method of clause 6, wherein the calendar visually distinguishes each pattern from another by color.
Clause 8. The method of any one of clauses 1 to 7, wherein the analysis of the plurality of daily glucose profiles comprises assessing a distance between the daily glucose profiles, wherein the distance comprises a mean absolute relative difference.
Clause 9. The method of clause 8, wherein first and second daily glucose profiles are grouped into a first pattern when the distance between the first and second daily glucose profiles is below a threshold.
Clause 10. The method of any one of clauses 1 to 9, further comprising determining one or more insights based on the report.
Clause 11. The method of clause 10, wherein the one or more insights comprises one or more recurring daily glucose patterns.
Clause 12. The method of clause 10, wherein the one or more insights comprises a time-of-day glucose variation, a day-to-day glucose variation, elevated glucose times, elevated glucose days, a weekday variation, a weekday-to-weekend variation, a medication dosing variation, a mealtime variation, an activity variation, or a combination thereof.
Clause 13. The method of clause 10, further comprising outputting, on a display of a receiver device, the one or more insights in a message to the user, a health care professional, or both.
Clause 14. The method of any one of clauses 1 to 13, wherein each of the daily glucose profiles comprises glucose data of the user over a 24-hour time window.
Clause 15. The method of any one of clauses 1 to 13, wherein each of the daily glucose profiles comprises glucose data of the user over less than a 24-hour time window.
Clause 16. The method of any one of clauses 1 to 15, wherein the analyzing is performed by a machine learning model.
Clause 17. A glucose pattern analysis system comprising:
an on-body unit configured to be worn on a skin surface of a user, the on-body unit comprising:
   a glucose sensor configured to measure glucose levels of the user, wherein the glucose sensor comprises a first portion arranged above the skin surface, and a second portion arranged below the skin surface and in contact with interstitial fluid of the user; and
   sensor electronics coupled to the glucose sensor and configured to wirelessly transmit glucose data; and
at least one processor in wireless communication with the on-body unit, the at least one processor coupled to at least one memory storing instructions that when executed cause the at least one processor to perform operations comprising:
   receiving therapy data of the user, wherein the therapy data comprises glucose data monitored by the on-body unit;
   analyzing a plurality of daily glucose profiles of the glucose data over an analysis period, optionally by a machine learning model;
   grouping the daily glucose profiles into two or more patterns based on the analysis, optionally performed by the machine learning model; and
   outputting a report comprising identification of one of the two or more patterns associated with each daily glucose profile.
Clause 18. The system of clause 17, wherein the report comprises a calendar with an indication of one of the two or more patterns associated with the daily glucose profile for each day on the calendar.
Clause 19. The system of clause 17 or clause 18, wherein the analysis is performed by a machine learning model, optionally wherein the machine learning model comprises unsupervised machine learning.
Clause 20. A computer-readable storage medium storing instructions which, when executed by at least one processor, cause the at least one processor to perform operations comprising:
receiving therapy data of a user, wherein the therapy data comprises glucose data monitored by a glucose monitoring device, wherein the glucose monitoring device comprises a first portion arranged above a skin surface of the user, and a second portion arranged below the skin surface and in contact with interstitial fluid of the user;
analyzing a plurality of daily glucose profiles of the glucose data over an analysis period, optionally by a machine learning model;
grouping the daily glucose profiles into two or more patterns based on the analysis, optionally performed by the machine learning model; and
outputting a report comprising identification of one of the two or more patterns associated with each daily glucose profile.
Clause 21. The method, system, or computer-readable storage medium of any one of the preceding clauses, wherein the report comprises a graph of the two or more patterns.
Clause 22. The method, system, or computer-readable storage medium of clause 21, wherein the graph maps the two or more patterns onto a plane and visually distinguishes each pattern from another.
Clause 23. The method, system, or computer-readable storage medium of clause 22, wherein the graph visually distinguishes each pattern from another by color.
Clause 24. The method, system, or computer-readable storage medium of clause 21, wherein the graph is based at least in part on a day-to-day glucose variation between each of the daily glucose profiles.
Clause 25. The method, system, or computer-readable storage medium of any one of the preceding clauses, wherein the therapy data comprises medication dosing data.
Clause 26. The method, system, or computer-readable storage medium of clause 25, the method or operations further comprising detecting, by a pen cap releasably coupleable to a manual insulin pen and in communication with the at least one processor, a dosing event of the manual insulin pen, wherein the dosing event is inferred from a decapping event of the pen cap from the manual insulin pen and a capping event of the pen cap to the manual insulin pen.
Clause 27. The method, system, or computer-readable storage medium of any one of the preceding clauses, wherein the therapy data comprises meal data.
Clause 28. The method, system, or computer-readable storage medium of clause 27, the method or operations further comprising detecting, by the at least one processor or a smart device in communication with the at least one processor, a meal event of the user.
Clause 29. The method, system, or computer-readable storage medium of any one of the preceding clauses, wherein the therapy data comprises activity data, exercise data, stress data, sleep data, location data, travel data, calendar data, or a combination thereof.
Clause 30. The method, system, or computer-readable storage medium of clause 29, the method or operations further comprising detecting, by the at least one processor or one or more smart devices in communication with the at least one processor, an activity event of the user, an exercise event of the user, a stress event of the user, a sleep event of the user, a location event of the user, a travel event of the user, or a combination thereof.
Clause 31. The method, system, or computer-readable storage medium of any one of the preceding clauses, wherein each of the daily glucose profiles comprises glucose data segments.
Clause 32. The method, system, or computer-readable storage medium of clause 31, wherein the glucose data segments comprise a breakfast time window, a lunch time window, a dinner time window, a snack time window, or a combination thereof.
Clause 33. The method, system, or computer-readable storage medium of any one of the preceding clauses, wherein the analyzing comprises generating, by the at least one processor, a distance matrix based on a distance between pairs of the daily glucose profiles.
Clause 34. The method, system, or computer-readable storage medium of clause 33, wherein the distance matrix is an N×N symmetric matrix representing the distance between each pair of N daily glucose profiles.
Clause 35. The method, system, or computer-readable storage medium of clause 33, wherein the distance is a weighted mean absolute difference.
Clause 36. The method, system, or computer-readable storage medium of clause 33, wherein the grouping comprises constructing a point set from the distance matrix, wherein each point of the point set represents a daily glucose profile.
Clause 37. The method, system, or computer-readable storage medium of clause 36, wherein the constructing the point set comprises multidimensional scaling of the distance matrix.
Clause 38. The method, system, or computer-readable storage medium of clause 37, wherein the multidimensional scaling comprises translating elements of the distance matrix to the point set in a plane such that such that a distance between two points of the point set is the distance between a corresponding pair of the daily glucose profiles.
Clause 39. The method, system, or computer-readable storage medium of clause 36, wherein the grouping further comprises performing cluster analysis on the point set.
Clause 40. The method, system, or computer-readable storage medium of clause 39, wherein the performing cluster analysis comprises performing k-means clustering.
Clause 41. The method, system, or computer-readable storage medium of clause 39, wherein the performing cluster analysis comprises performing a Gaussian mixture model.
Clause 42. The method, system, or computer-readable storage medium of clause 39, wherein the performing cluster analysis comprises utilizing a metric to identify the two or more patterns from each other.
Clause 43. The method, system, or computer-readable storage medium of clause 42, wherein the metric comprises a distance reduction ratio metric based on a separation between each pattern.
Clause 44. The method, system, or computer-readable storage medium of clause 43, wherein the distance reduction ratio metric is defined as a ratio between an average inter-pattern distance to an average intra-pattern distance:

$$R = \frac{{avg}_{( i , j ) ∈ S 1} d ( i , j )}{{avg}_{u ∈ S 1 and v ∈ S 2} {d}^{′} ( u , v )}$$

where d(i, j) represents the inter-pattern distance between two analyte profiles i and j, both belonging to the same pattern S1, and d′(u, v) represents the intra-pattern distance of two analyte profiles u and v, from two different patterns S1 and S2 respectively.
Clause 45. The method, system, or computer-readable storage medium of clause 43, wherein the distance reduction ratio metric comprises a threshold above which the two or more patterns are identified.
Clause 46. The method, system, or computer-readable storage medium of clause 45, wherein the threshold is at least 20%.
Clause 47. The method, system, or computer-readable storage medium of clause 42, wherein the metric comprises a meal metric based on a separation between each pattern in relation to meal data of the user.
Clause 48. The method, system, or computer-readable storage medium of clause 42 or clause 47, wherein the metric comprises a medication dosing metric based on a separation between each pattern in relation to medication dosing data of the user.
Clause 49. The method, system, or computer-readable storage medium of any one of the preceding clauses, wherein the analyzing and the grouping comprises utilizing the machine learning model to compare the daily glucose profiles and identify the two or more patterns.
Clause 50. The method, system, or computer-readable storage medium of any one of the preceding clauses, wherein the machine learning model comprises unsupervised machine learning.
Clause 51. The method, system, or computer-readable storage medium of any one of the preceding clauses, wherein the machine learning model comprises supervised machine learning.
Clause 52. The method, system, or computer-readable storage medium of any one of the preceding clauses, the method or operations further comprising correlating the two or more patterns to one or more user parameters based on one or more metrics.
Clause 53. The method, system, or computer-readable storage medium of clause 52, wherein the one or more user parameters comprises medication dosing data statistics, meal data statistics, activity data statistics, exercise data statistics, stress data statistics, sleep data statistics, location data statistics, travel data statistics, calendar data statistics, daily routine statistics, or a combination thereof.
Clause 54. The method, system, or computer-readable storage medium of clause 52 or clause 53, wherein the one or more metrics comprises a statistical metric, a distance reduction ratio metric, a meal metric, a medication dosing metric, or a combination thereof.
Clause 55. The method, system, or computer-readable storage medium of any one of the preceding clauses, the method or operations further comprising providing a recommendation to the user or a health care professional based at least in part on the two or more patterns.
Clause 56. The method, system, or computer-readable storage medium of any one of clauses 52 to 54, the method or operations further comprising providing a recommendation to the user or a health care professional based on a correlation of the two or more patterns to the one or more user parameters.
Clause 57. The method, system, or computer-readable storage medium of any one of the preceding clauses, wherein the analysis period is in a range from 3 days to 14 days.
Clause 58. The method, system, or computer-readable storage medium of clause 57, wherein the grouping is limited to no greater than three patterns.
Clause 59. The method, system, or computer-readable storage medium of any one of the preceding clauses, wherein the analysis period is at least 14 days.
Clause 60. The method, system, or computer-readable storage medium of any one of the preceding clauses, wherein the analysis period is in a range from 3 days to 28 days.
Clause 61. The method, system, or computer-readable storage medium of any one of the preceding clauses, wherein the analysis period is at least 28 days.
Clause 62. The method, system, or computer-readable storage medium of any one of the preceding clauses, wherein the analysis period is in a range from 30 days to 90 days.
Clause 63. The method, system, or computer-readable storage medium of any one of the preceding clauses, wherein the analysis period is at least 45 days.
Clause 64. The system of any one of clauses 16 to 19, further comprising a pen cap releasably coupleable to a manual insulin pen and configured to detect a dosing event of the manual insulin pen, wherein the dosing event is inferred from a decapping event of the pen cap from the manual insulin pen and a capping event of the pen cap to the manual insulin pen.
Clause 65. The system of any one of clauses 16 to 19 or clause 64, further comprising a smart device in communication with the at least one processor and configured to detect a meal event of the user.
Clause 66. The system of any one of clauses 16 to 19, clause 64, or clause 65, further comprising one or more smart devices in communication with the at least one processor and configured to detect an activity event of the user, an exercise event of the user, a stress event of the user, a sleep event of the user, a location event of the user, a travel event of the user, or a combination thereof.
Clause 67. The method, system, or computer-readable storage medium of any one of the preceding clauses, wherein the analysis of the daily glucose profiles comprises assessing a distance between the daily glucose profiles, wherein the distance comprises a mean absolute difference (MAD).
Clause 68. The method, system, or computer-readable storage medium of any one of the preceding clauses, wherein the analysis of the daily glucose profiles comprises assessing a distance between the daily glucose profiles, wherein the distance comprises a weighted MAD (WMAD).
Clause 69. The method, system, or computer-readable storage medium of any one of the preceding clauses, the method or operations further comprising providing one or more recommendations to adjust a therapy of the user based at least in part on the grouping of the two or more patterns.
Clause 70. The method, system, or computer-readable storage medium of any one of the preceding clauses, the method or operations further comprising providing one or more alerts or alarms associated with therapy of the user based at least in part on the grouping of the two or more patterns.
Clause 71. A method of identifying and reporting patterns of analyte data, the method comprising:
monitoring, by an analyte monitoring device, analyte levels of a user, wherein the analyte monitoring device comprises a first portion arranged above a skin surface of the user, and a second portion arranged below the skin surface and in contact with interstitial fluid of the user;
receiving, by at least one processor, therapy data of the user, wherein the therapy data comprises analyte data monitored by the analyte monitoring device;
analyzing, by the at least one processor, analyte profiles of the analyte data over an analysis period by a model, optionally by a machine learning model;
identifying, by the at least one processor, each of the analyte profiles as corresponding to a pattern of two or more patterns based on the analysis performed by the model, optionally performed by the machine learning model; and outputting, on a display device in communication with the at least one processor, a report comprising identification of one of the two or more patterns associated with each analyte profile.
Clause 72. The method of clause 71, wherein the report comprises a display of each analyte profile.
Clause 73. The method of clause 72, wherein the display overlays the two or more patterns over the analysis period and visually distinguishes the patterns from one another.
Clause 74. The method of clause 73, wherein the display visually distinguishes the patterns from one another by color.
Clause 75. The method of any one of clauses 71 to 74, wherein the report comprises a calendar with an indication of an identified pattern of the two or more patterns for each day on the calendar.
Clause 76. The method of clause 75, wherein the calendar separates the two or more patterns into corresponding calendar days and visually distinguishes the patterns from one another.
Clause 77. The method of clause 76, wherein the calendar visually distinguishes the patterns from one another by color.
Clause 78. The method of any one of clauses 71 to 77, wherein the analysis of the analyte profiles comprises assessing a distance between the analyte profiles, wherein the distance comprises a mean absolute relative difference.
Clause 79. The method of clause 78, wherein first and second analyte profiles are grouped into a first pattern when the distance between the first and second analyte profiles is at or below a threshold.
Clause 80. The method of any one of clauses 71 to 79, further comprising determining one or more insights based on the report.
Clause 81. The method of clause 80, wherein the one or more insights comprises one or more recurring analyte patterns, optionally one or more recurring glucose patterns.
Clause 82. The method of clause 80, wherein the one or more insights comprises a time-of-day analyte variation, a day-to-day analyte variation, elevated analyte times, elevated analyte days, a weekday analyte variation, a weekday-to-weekend analyte variation, a medication dosing variation, a mealtime variation, an activity variation, or a combination thereof.
Clause 83. The method of clause 80, further comprising outputting, on a display on a receiver device, the one or more insights in a message to the user, a health care professional, or both.
Clause 84. The method of any one of clauses 71 to 83, wherein each of the analyte profiles comprises analyte data of the user that is collected over a 24-hour time window.
Clause 85. The method of any one of clauses 71 to 83, wherein each of the analyte profiles comprises analyte data of the user that is collected over less than a 24-hour time window.
Clause 86. An analyte pattern analysis system comprising:
an on-body unit configured to be worn on a skin surface of a user, the on-body unit comprising:
   a analyte sensor configured to measure analyte levels of the user, wherein the analyte sensor comprises a first portion arranged above the skin surface, and a second portion arranged below the skin surface and in contact with interstitial fluid of the user; and
   sensor electronics coupled to the analyte sensor and configured to wirelessly transmit analyte data; and
at least one processor in wireless communication with the on-body unit, the at least one processor coupled to at least one memory storing instructions that when executed cause the at least one processor to perform operations comprising:
   receiving therapy data of the user, wherein the therapy data comprises analyte data monitored by the on-body unit;
   analyzing analyte profiles of the analyte data over an analysis period by a model, optionally by a machine learning model;
   identifying each of the analyte profiles as corresponding to a pattern of two or more patterns based on the analysis performed by the model, optionally performed by the machine learning model; and
   outputting a report comprising identification of one of the two or more patterns associated with each analyte profile.
Clause 87. The system of clause 86, wherein the report comprises a display of each analyte profile.
Clause 88. The system of clause 86 or clause 87, wherein the report comprises a calendar with an indication of an identified pattern of the two or more patterns for each day on the calendar.
Clause 89. The system of any one of clauses 86 to 88, wherein the model comprises a machine learning model, optionally an unsupervised machine learning model.
Clause 90. A computer-readable storage medium storing instructions which, when executed by at least one processor, cause the at least one processor to perform operations comprising:
receiving therapy data of a user, wherein the therapy data comprises analyte data monitored by a analyte monitoring device, wherein the analyte monitoring device comprises a first portion arranged above a skin surface of the user, and a second portion arranged below the skin surface and in contact with interstitial fluid of the user;
analyzing analyte profiles of the analyte data over an analysis period by a model, optionally by a machine learning model;
identifying each of the analyte profiles as corresponding to a pattern of two or more patterns based on the analysis performed by the model, optionally performed by the machine learning model; and outputting a report comprising identification of one of the two or more patterns associated with each analyte profile.
Clause 91. The method, system, or computer-readable storage medium of any one of clauses 71 to 90, wherein the analyte profiles comprise daily analyte profiles.
Clause 92. The method, system, or computer-readable storage medium of any one of clauses 71 to 91, wherein the analyte profiles comprise glucose profiles.
Clause 93. The method, system, or computer-readable storage medium of any one of the preceding clauses, wherein the analyte profiles comprise ketones profiles.
Clause 94. The method, system, or computer-readable storage medium of any one of the preceding clauses, wherein the analyte profiles comprise lactate profiles.
Clause 95. The method, system, or computer-readable storage medium of any one of clauses 71 to 94, wherein the model comprises a machine learning model.
Clause 96. The method, system, or computer-readable storage medium of any one of clauses 71 to 95, wherein the model comprises an unsupervised machine learning model.
Clause 97. The method, system, or computer-readable storage medium of any one of the preceding clauses, the method or operations further comprising outputting one or more recommendations, notifications, and/or alerts based at least in part on a time-of-day analyte variation determined by the analysis or model.
Clause 98. The method, system, or computer-readable storage medium of clause 97, wherein the one or more recommendations comprises a time-of-day therapy adjustment (e.g., increase insulin dose amount at lunch, decrease exercise in the evening, etc.).
Clause 99. The method, system, or computer-readable storage medium of any one of the preceding clauses, the method or operations further comprising outputting one or more recommendations, notifications, and/or alerts based at least in part on a day-to-day analyte variation determined by the analysis or model.
Clause 100. The method, system, or computer-readable storage medium of clause 99, wherein the one or more recommendations comprises a day-to-day therapy adjustment (e.g., increase insulin dose amount on Tuesdays, eat more for breakfast on Fridays, etc.).
Clause 101. The method, system, or computer-readable storage medium of any one of the preceding clauses, the method or operations further comprising outputting one or more recommendations, notifications, and/or alerts based at least in part on elevated analyte times determined by the analysis or model.
Clause 102. The method, system, or computer-readable storage medium of clause 101, wherein the one or more recommendations comprises a therapy adjustment for certain times (e.g., eat more during 10:00 AM to 12:00 PM, avoid strenuous exercise during 5:00 PM to 7:00 PM, etc.).
Clause 103. The method, system, or computer-readable storage medium of any one of the preceding clauses, the method or operations further comprising outputting one or more recommendations, notifications, and/or alerts based at least in part on elevated analyte days determined by the analysis or model.
Clause 104. The method, system, or computer-readable storage medium of any one of clause 103, wherein the one or more recommendations comprises a therapy adjustment for certain days (e.g., increase insulin dose amount on Friday mornings, avoid exercise on Sunday evenings, etc.).
Clause 105. The method, system, or computer-readable storage medium of any one of the preceding clauses, the method or operations further comprising outputting one or more recommendations, notifications, and/or alerts based at least in part on a weekday analyte variation determined by the analysis or model.
Clause 106. The method, system, or computer-readable storage medium of any one of clause 105, wherein the one or more recommendations comprises a weekday therapy adjustment (e.g., eat less for dinner on Tuesdays, eat more for lunch on Thursdays, etc.).
Clause 107. The method, system, or computer-readable storage medium of any one of the preceding clauses, the method or operations further comprising outputting one or more recommendations, notifications, and/or alerts based at least in part on a weekday-to-weekend analyte variation determined by the analysis or model.
Clause 108. The method, system, or computer-readable storage medium of clause 107, wherein the one or more recommendations comprises a weekday-to-weekend therapy adjustment (e.g., outlier for dinner on Friday, increase insulin dose amount for lunch on Saturdays and Sundays, etc.).
Clause 109. The method, system, or computer-readable storage medium of any one of the preceding clauses, the method or operations further comprising outputting one or more recommendations, notifications, and/or alerts based at least in part on a medication dosing variation determined by the analysis or model.
Clause 110. The method, system, or computer-readable storage medium of clause 109, wherein the one or more recommendations comprises a medication dosing adjustment (e.g., increase insulin dose amount for Monday mornings, decrease insulin dose amount for Thursday evenings, decrease GLP-1 dose amounts on Fridays, etc.).
Clause 111. The method, system, or computer-readable storage medium of any one of the preceding clauses, the method or operations further comprising outputting one or more recommendations, notifications, and/or alerts based at least in part on a mealtime variation determined by the analysis or model.
Clause 112. The method, system, or computer-readable storage medium of clause 111, wherein the one or more recommendations comprises a mealtime adjustment (e.g., eat more for dinner on Mondays, eat less for lunch on Saturdays, etc.).
Clause 113. The method, system, or computer-readable storage medium of any one of the preceding clauses, the method or operations further comprising outputting one or more recommendations, notifications, and/or alerts based at least in part on an activity variation determined by the analysis or model.
Clause 114. The method, system, or computer-readable storage medium of clause 113, wherein the one or more recommendations comprises an activity adjustment (e.g., avoid strenuous exercise on Sunday evenings, increase exercise on Saturday afternoons, get more sleep on Wednesdays, etc.)
Clause 115. A method of generating titration profiles for adaptive dose guidance, the method comprising:
monitoring, by a glucose monitoring device, glucose levels of a user, wherein the glucose monitoring device comprises a first portion arranged above a skin surface of the user, and a second portion arranged below the skin surface and in contact with interstitial fluid of the user;
receiving, by at least one processor in communication with the glucose monitoring device, user data of the user, wherein the user data comprises glucose data monitored by the glucose monitoring device;
analyzing, by the at least one processor, a plurality of glucose profiles of the glucose data over an analysis period;
grouping, by the at least one processor, the plurality of glucose profiles into two or more patterns based on the analysis;
generating, by the at least one processor, a plurality of titration profiles, wherein each titration profile corresponds to one of the two or more patterns identified;
adjusting, by the at least one processor, one or more insulin therapy settings of a medication delivery device based on a titration profile of the plurality of titration profiles;
outputting, on a display, a dose recommendation based on the titration profile.
Clause 116. The method of clause 115, wherein:
the two or more patterns identified comprises weekdays and weekends, and
the plurality of titration profiles comprises a first titration profile corresponding to weekdays and a second titration profile corresponding to weekends.
Clause 117. The method of clause 115 or clause 116, wherein:
the two or more patterns identified comprises a day shift and a night shift, and
the plurality of titration profiles comprises a first titration profile corresponding to a day shift and a second titration profile corresponding to a night shift.
Clause 118. The method of clause 117, wherein the second titration profile is shifted later in time relative to the first titration profile.
Clause 119. The method of any one of clauses 115 to 118, wherein:
the two or more patterns identified comprises not traveling and traveling, and the plurality of titration profiles comprises a first titration profile corresponding to not traveling and a second titration profile corresponding to traveling.
Clause 120. The method of clause 119, wherein the second titration profile is shifted in time relative to the first titration profile based on a current location of the user.
Clause 121. The method of any one of clauses 115 to 120, further comprising generating, by the at least one processor, a second titration profile based on one of the plurality of titration profiles when a variability of the titration profile is above a threshold.
Clause 122. The method of any one of clauses 115 to 121, wherein the analyzing is performed by a machine learning model.
Clause 123. The method of clause 122, wherein the machine learning model comprises an unsupervised machine learning model performing cluster analysis.
Clause 124. The method of any one of clauses 115 to 123, wherein the analyzing is performed by a generative artificial intelligence (AI) model.
Clause 125. The method of clause 124, further comprising prompting the generative AI model to determine whether the plurality of glucose profiles are correlated to weekdays or weekends.
Clause 126. The method of clause 124 or clause 125, further comprising prompting the generative AI model to determine whether the plurality of glucose profiles are correlated to a day shift or a night shift.
Clause 127. The method of any one of clauses 124 to 126, further comprising prompting the generative AI model to determine whether the plurality of glucose profiles are correlated to not traveling or traveling.
Clause 128. The method of any one of clauses 124 to 127, further comprising prompting the generative AI model to determine whether the plurality of glucose profiles are correlated to a first type of user data.
Clause 129. The method of clause 128, further comprising prompting the generative AI model to determine whether the two or more patterns are correlated to a second type of user data.
Clause 130. The method of clause 129, wherein the prompting is performed by a large language model (LLM) that compares the two or more patterns to the second type of user data to determine a level of correlation.
Clause 131. A pattern analysis system comprising:
a glucose monitoring device configured to be worn on a skin surface of a user, the glucose monitoring device comprising:
   a glucose sensor configured to measure glucose levels of the user, wherein the glucose sensor comprises a first portion arranged above the skin surface, and a second portion arranged below the skin surface and in contact with interstitial fluid of the user; and
   sensor electronics coupled to the glucose sensor and configured to wirelessly transmit glucose data; and
at least one processor in communication with the glucose monitoring device, the at least one processor coupled to at least one memory storing instructions that when executed cause the at least one processor to perform operations comprising:
   receiving user data of the user, wherein the user data comprises glucose data monitored by the glucose monitoring device;
   analyzing a plurality of glucose profiles of the glucose data over an analysis period;
   grouping the plurality of glucose profiles into two or more patterns based on the analysis;
   generating a plurality of titration profiles, wherein each titration profile corresponds to one of the two or more patterns identified;
   adjusting one or more insulin therapy settings of a medication delivery device based on a titration profile of the plurality of titration profiles; and
   outputting on a display a dose recommendation based on the titration profile.
Clause 132. The system of clause 131, wherein the analysis is performed by a machine learning model, optionally wherein the machine learning model comprises an unsupervised machine learning model performing cluster analysis.
Clause 133. The system of clause 131, wherein the analysis is performed by a generative artificial intelligence (AI) model.
Clause 134. A computer-readable storage medium storing instructions which, when executed by at least one processor, cause the at least one processor to perform operations comprising:
receiving user data of a user, wherein the user data comprises glucose data monitored by a glucose monitoring device, wherein the glucose monitoring device comprises a first portion arranged above a skin surface of the user, and a second portion arranged below the skin surface and in contact with interstitial fluid of the user;
analyzing a plurality of glucose profiles of the glucose data over an analysis period;
grouping the glucose profiles into two or more patterns based on the analysis;
generating a plurality of titration profiles, wherein each titration profile corresponds to one of the two or more patterns identified;
adjusting one or more insulin therapy settings of a medication delivery device based on a titration profile of the plurality of titration profiles; and
outputting on a display a dose recommendation based on the titration profile.
Clause 135. A method of generating titration profiles for adaptive dose guidance for pregnancy, the method comprising:
monitoring, by a glucose monitoring device, glucose levels of a user, wherein the glucose monitoring device comprises a first portion arranged above a skin surface of the user, and a second portion arranged below the skin surface and in contact with interstitial fluid of the user;
receiving, by at least one processor in communication with the glucose monitoring device, user data of the user, wherein the user data comprises glucose data monitored by the glucose monitoring device and pregnancy data regarding whether the user is pregnant or not pregnant;
analyzing, by the at least one processor, a plurality of glucose profiles of the glucose data and the pregnancy data over an analysis period;
grouping, by the at least one processor, the plurality of glucose profiles into a first pattern corresponding to the user being pregnant and a second pattern corresponding to the user not being pregnant based on the analysis; and
generating, by the at least one processor, a first titration profile corresponding to the user being pregnant based on the first pattern and a second titration profile corresponding to the user not being pregnant based on the second pattern.
Clause 136. The method of clause 135, further comprising dynamically updating, by the at least one processor, the first titration profile in real time based on a variability of the first titration profile over time.
Clause 137. The method of clause 135 or clause 136, wherein the first titration profile has a higher insulin dose range than the second titration profile.
Clause 138. The method of any one of clauses 135 to 137, further comprising outputting a query to the user regarding whether the user is pregnant or not pregnant.
Clause 139. The method of clause 138, wherein the pregnancy data is received from the user based on a response to the query.
Clause 140. The method of any one of clauses 135 to 139, wherein the analyzing is performed by a generative artificial intelligence (AI) model.
Clause 141. The method of clause 140, further comprising prompting the generative AI model to determine whether the plurality of glucose profiles are correlated to the user being pregnant or the user not being pregnant.
Clause 142. A pattern analysis system for adaptive dose guidance for pregnancy, the system comprising:
a glucose monitoring device configured to be worn on a skin surface of a user, the glucose monitoring device comprising:
   a glucose sensor configured to measure glucose levels of the user, wherein the glucose sensor comprises a first portion arranged above the skin surface, and a second portion arranged below the skin surface and in contact with interstitial fluid of the user; and
   sensor electronics coupled to the glucose sensor and configured to wirelessly transmit glucose data; and
at least one processor in communication with the glucose monitoring device, the at least one processor coupled to at least one memory storing instructions that when executed cause the at least one processor to perform operations comprising:
   receiving user data of the user, wherein the user data comprises glucose data monitored by the glucose monitoring device and pregnancy data regarding whether the user is pregnant or not pregnant;
   analyzing a plurality of glucose profiles of the glucose data and the pregnancy data over an analysis period;
   grouping the plurality of glucose profiles into first pattern corresponding to the user being pregnant and a second pattern corresponding to the user not being pregnant based on the analysis; and
   generating a first titration profile corresponding to the user being pregnant based on the first pattern and a second titration profile corresponding to the user not being pregnant based on the second pattern.
Clause 143. The system of clause 142, wherein the operations further include dynamically updating the first titration profile in real time based on a variability of the first titration profile over time.
Clause 144. The system of clause 142 or clause 143, wherein the first titration profile has a higher insulin dose range than the second titration profile.
Clause 145. A method of generating titration profiles for adaptive dose guidance for menstruation, the method comprising:
monitoring, by a glucose monitoring device, glucose levels of a user, wherein the glucose monitoring device comprises a first portion arranged above a skin surface of the user, and a second portion arranged below the skin surface and in contact with interstitial fluid of the user;
receiving, by at least one processor in communication with the glucose monitoring device, user data of the user, wherein the user data comprises glucose data monitored by the glucose monitoring device and menstruation data regarding whether the user is menstruating or not menstruating;
analyzing, by the at least one processor, a plurality of glucose profiles of the glucose data and the menstruation data over an analysis period;
grouping, by the at least one processor, the plurality of glucose profiles into a first pattern corresponding to the user menstruating and a second pattern corresponding to the user not menstruating based on the analysis; and
generating, by the at least one processor, a first titration profile corresponding to the user menstruating based on the first pattern and a second titration profile corresponding to the user not menstruating based on the second pattern.
Clause 146. The method of clause 145, further comprising dynamically updating, by the at least one processor, the first titration profile in real time based on a variability of the first titration profile over time.
Clause 147. The method of clause 145 or clause 146, wherein the first titration profile has a higher insulin dose range and a lower insulin sensitivity factor (ISF) than the second titration profile.
Clause 148. The method of any one of clauses 145 to 147, further comprising outputting a query to the user regarding whether the user is menstruating or not menstruating.
Clause 149. The method of clause 148, wherein the menstruation data is received from the user based on a response to the query.
Clause 150. The method of any one of clauses 145 to 149, wherein the analyzing is performed by a generative artificial intelligence (AI) model.
Clause 151. The method of clause 150, further comprising prompting the generative AI model to determine whether the plurality of glucose profiles are correlated to the user menstruating or the user not menstruating.
Clause 152. A pattern analysis system for adaptive dose guidance for menstruation, the system comprising:
a glucose monitoring device configured to be worn on a skin surface of a user, the glucose monitoring device comprising:
   a glucose sensor configured to measure glucose levels of the user, wherein the glucose sensor comprises a first portion arranged above the skin surface, and a second portion arranged below the skin surface and in contact with interstitial fluid of the user; and
   sensor electronics coupled to the glucose sensor and configured to wirelessly transmit glucose data; and
at least one processor in communication with the glucose monitoring device, the at least one processor coupled to at least one memory storing instructions that when executed cause the at least one processor to perform operations comprising:
   receiving user data of the user, wherein the user data comprises glucose data monitored by the glucose monitoring device and menstruation data regarding whether the user is menstruating or not menstruating;
   analyzing a plurality of glucose profiles of the glucose data and the menstruation data over an analysis period;
   grouping the plurality of glucose profiles into first pattern corresponding to the user menstruating and a second pattern corresponding to the user not menstruating based on the analysis; and
   generating a first titration profile corresponding to the user menstruating based on the first pattern and a second titration profile corresponding to the user not menstruating based on the second pattern.
Clause 153. The system of clause 152, wherein the operations further include dynamically updating the first titration profile in real time based on a variability of the first titration profile over time.
Clause 154. The system of clause 152, wherein the first titration profile has a higher insulin dose range and a lower insulin sensitivity factor (ISF) than the second titration profile.
Clause 155. A method of generating titration profiles for adaptive dose guidance for illness, the method comprising:
monitoring, by a glucose monitoring device, glucose levels of a user, wherein the glucose monitoring device comprises a first portion arranged above a skin surface of the user, and a second portion arranged below the skin surface and in contact with interstitial fluid of the user;
receiving, by at least one processor in communication with the glucose monitoring device, user data of the user, wherein the user data comprises glucose data monitored by the glucose monitoring device and illness data regarding whether the user is ill or not ill;
analyzing, by the at least one processor, a plurality of glucose profiles of the glucose data and the illness data over an analysis period;
grouping, by the at least one processor, the plurality of glucose profiles into a first pattern corresponding to the user being ill and a second pattern corresponding to the user not being ill based on the analysis; and
generating, by the at least one processor, a first titration profile corresponding to the user being ill based on the first pattern and a second titration profile corresponding to the user not being ill based on the second pattern.
Clause 156. The method of clause 155, further comprising dynamically updating, by the at least one processor, the first titration profile in real time based on a variability of the first titration profile over time.
Clause 157. The method of clause 155 or clause 156, wherein the first titration profile has a higher insulin dose range than the second titration profile.
Clause 158. The method of any one of clauses 155 to 157, wherein the first titration profile has a lower insulin dose range than the second titration profile.
Clause 159. The method of any one of clauses 155 to 158, further comprising outputting a query to the user regarding whether the user is ill or not ill.
Clause 160. The method of clause 159, wherein the illness data is received from the user based on a response to the query.
Clause 161. The method of any one of clauses 155 to 160, wherein the illness data comprises a type of illness.
Clause 162. The method of any one of clauses 155 to 161, wherein the analyzing is performed by a generative artificial intelligence (AI) model.
Clause 163. The method of clause 162, further comprising prompting the generative AI model to determine whether the plurality of glucose profiles are correlated to the user being ill or the user not being ill.
Clause 164. A pattern analysis system for adaptive dose guidance for illness, the system comprising:
a glucose monitoring device configured to be worn on a skin surface of a user, the glucose monitoring device comprising:
   a glucose sensor configured to measure glucose levels of the user, wherein the glucose sensor comprises a first portion arranged above the skin surface, and a second portion arranged below the skin surface and in contact with interstitial fluid of the user; and
   sensor electronics coupled to the glucose sensor and configured to wirelessly transmit glucose data; and
at least one processor in communication with the glucose monitoring device, the at least one processor coupled to at least one memory storing instructions that when executed cause the at least one processor to perform operations comprising:
   receiving user data of the user, wherein the user data comprises glucose data monitored by the glucose monitoring device and illness data regarding whether the user is ill or not ill;
   analyzing a plurality of glucose profiles of the glucose data and the illness data over an analysis period;
   grouping the plurality of glucose profiles into first pattern corresponding to the user being ill and a second pattern corresponding to the user not being ill based on the analysis; and
   generating a first titration profile corresponding to the user being ill based on the first pattern and a second titration profile corresponding to the user not being ill based on the second pattern.
Clause 165. A method of identifying and reporting patterns of glucose data, the method comprising:
monitoring, by a glucose monitoring device, glucose levels of a user, wherein the glucose monitoring device comprises a first portion arranged above a skin surface of the user, and a second portion arranged below the skin surface and in contact with interstitial fluid of the user;
receiving, by at least one processor in communication with the glucose monitoring device, therapy data of the user, wherein the therapy data comprises glucose data monitored by the glucose monitoring device;
analyzing, by the at least one processor, a plurality of daily glucose profiles of the glucose data over an analysis period;
grouping, by the at least one processor, the daily glucose profiles into two or more patterns based on the analysis;
outputting, on a display device in communication with the at least one processor, a report comprising identification of one of the two or more patterns associated with each daily glucose profile, wherein the report comprises:
   a display of each daily glucose profile, wherein the display overlays the two or more patterns over the analysis period and visually distinguishes each of the two or more patterns from another by color, and
   a calendar with an indication of one of the two or more patterns associated with the daily glucose profile for each day on the calendar.
Clause 166. The method, system, or computer-readable storage medium of any one of the preceding clauses, the method or operations further comprising generating, by the at least one processor, a plurality of titration profiles, wherein each titration profile corresponds to one of the two or more patterns identified.
Clause 167. The method, system, or computer-readable storage medium of any one of the preceding clauses, the method or operations further comprising adjusting, by the at least one processor, one or more insulin therapy settings of a medication delivery device based on a titration profile of the plurality of titration profiles.
Clause 168. The method, system, or computer-readable storage medium of any one of the preceding clauses, wherein the one or more insulin therapy settings comprises one or more of a dose amount, an insulin sensitivity factor (ISF), or a carbohydrate-to-insulin ratio (CR).
Clause 169. The method, system, or computer-readable storage medium of any one of the preceding clauses, the method or operations further comprising outputting, on the display device, a dose recommendation based on the titration profile.
Clause 170. The method, system, or computer-readable storage medium of any one of the preceding clauses, further comprising updating, by the at least one processor, the titration profile.
Clause 171. The method of clause 170, wherein the updating is performed in real time based on glucose data monitored over a second analysis period.
Clause 172. The method of clause 170, wherein the updating is based on a variability of one or more parameters of the titration profile exceeding a threshold.
Clause 173. The method of any one of clauses 170-172, wherein the one or more parameters of the titration profile comprises a dose amount, an insulin sensitivity factor (ISF), a carbohydrate-to-insulin ratio (CR), or a combination thereof.
Clause 174. The method, system, or computer-readable storage medium of any one of the preceding clauses, further comprising updating, by the at least one processor, the first titration profile.
Clause 175. The method of clause 174, wherein the updating is performed in real time based on glucose data and pregnancy data monitored over a second analysis period.
Clause 176. The method of clause 174, wherein the updating is based on a variability of one or more parameters of the first titration profile exceeding a threshold.
Clause 177. The method of any one of clauses 174-176, wherein the one or more parameters of the first titration profile comprises a dose amount, an insulin sensitivity factor (ISF), a carbohydrate-to-insulin ratio (CR), a total body weight of the user, or a combination thereof.
Clause 178. The method, system, or computer-readable storage medium of any one of the preceding clauses, further comprising updating, by the at least one processor, the first titration profile.
Clause 179. The method of clause 178, wherein the updating is performed in real time based on glucose data and menstruation data monitored over a second analysis period.
Clause 180. The method of clause 178, wherein the updating is based on a variability of one or more parameters of the first titration profile exceeding a threshold.
Clause 181. The method of any one of clauses 178-180, wherein the one or more parameters of the first titration profile comprises a dose amount, an insulin sensitivity factor (ISF), a carbohydrate-to-insulin ratio (CR), a current menstrual phase of the user, or a combination thereof.
Clause 182. The method, system, or computer-readable storage medium of any one of the preceding clauses, further comprising updating, by the at least one processor, the first titration profile.
Clause 183. The method of clause 182, wherein the updating is performed in real time based on glucose data and illness data monitored over a second analysis period.
Clause 184. The method of clause 182, wherein the updating is based on a variability of one or more parameters of the first titration profile exceeding a threshold.
Clause 185. The method of any one of clauses 182-184, wherein the one or more parameters of the first titration profile comprises a dose amount, an insulin sensitivity factor (ISF), a carbohydrate-to-insulin ratio (CR), a body temperature of the user, or a combination thereof.
Clause 186. A method of triggering an update to a titration profile for adaptive dose guidance, the method comprising:
monitoring, by a glucose monitoring device, glucose levels of a user, wherein the glucose monitoring device comprises a first portion arranged above a skin surface of the user, and a second portion arranged below the skin surface and in contact with interstitial fluid of the user;
receiving, by at least one processor in communication with the glucose monitoring device, user data of the user, wherein the user data comprises glucose data monitored by the glucose monitoring device; and
updating, by the at least one processor, a titration profile of the user based on a variability of one or more parameters of the titration profile exceeding a threshold.
Clause 187. The method of clause 186, wherein the one or more parameters of the titration profile comprises a dose amount, an insulin sensitivity factor (ISF), a carbohydrate-to-insulin ratio (CR), or a combination thereof.
Clause 188. The method of clause 186, wherein the updating comprises updating based on a change in user data.
Clause 189. The method of any one of clauses 186-188, wherein the change in user data comprises a change in pregnancy data, a change in menstruation data, a change in illness data, or a combination thereof.

1. A method of identifying and reporting patterns of glucose data, the method comprising:
monitoring, by a glucose monitoring device, glucose levels of a user, wherein the glucose monitoring device comprises a first portion arranged above a skin surface of the user, and a second portion arranged below the skin surface and in contact with interstitial fluid of the user;
receiving, by at least one processor in communication with the glucose monitoring device, therapy data of the user, wherein the therapy data comprises glucose data monitored by the glucose monitoring device;
analyzing, by the at least one processor, a plurality of daily glucose profiles of the glucose data over an analysis period, wherein the analyzing is performed by a machine learning model performing cluster analysis of the plurality of daily glucose profiles;
grouping, by the at least one processor, the daily glucose profiles into two or more patterns based on the analysis;
outputting, on a display device in communication with the at least one processor, a report comprising identification of one of the two or more patterns associated with each daily glucose profile, wherein the report comprises:
a display of each daily glucose profile, wherein the display overlays the two or more patterns over the analysis period and visually distinguishes each of the two or more patterns from another by color, and
a calendar with an indication of one of the two or more patterns associated with the daily glucose profile for each day on the calendar.

2. The method of claim 1, further comprising generating, by the at least one processor, a plurality of titration profiles, wherein each titration profile corresponds to one of the two or more patterns identified.

3. The method of claim 2, further comprising adjusting, by the at least one processor, one or more insulin therapy settings of a medication delivery device based on a titration profile of the plurality of titration profiles.

4. The method of claim 3, wherein the one or more insulin therapy settings comprises one or more of a dose amount, an insulin sensitivity factor (ISF), or a carbohydrate-to-insulin ratio (CR).

5. The method of claim 3, further comprising outputting, on the display device, a dose recommendation based on the titration profile.

6. The method of claim 1, wherein the calendar separates the two or more patterns into corresponding calendar days and visually distinguishes each pattern from another.

7. The method of claim 6, wherein the calendar visually distinguishes each pattern from another by color.

8. The method of claim 1, wherein the analysis of the plurality of daily glucose profiles comprises assessing a distance between the daily glucose profiles, wherein the distance comprises a mean absolute relative difference.

9. The method of claim 8, wherein first and second daily glucose profiles are grouped into a first pattern when the distance between the first and second daily glucose profiles is below a threshold.

10-90. (canceled)

91. The method of claim 1, wherein the analyzing comprises determining whether the plurality of daily glucose profiles are correlated to a first type of user data.
